(19) United States
(12) Patent Application Publication
(10) Pub. No.: US 2026/0103455 A1
(43) Pub. Date: Apr. 16, 2026

(54) ULK3 INHIBITORS AND USES THEREOF

(71) Applicant: H. LEE MOFFITT CANCER CENTER AND RESEARCH INSTITUTE, INC., Tampa, FL (US)

(72) Inventors: Conor C. LYNCH, Tampa, FL (US); Nicholas J. LAWRENCE, Lutz, FL (US); Harshani R. LAWRENCE, Lutz, FL (US); Marilena TAURO, Tampa, FL (US)

(21) Appl. No.: 19/102,552

(22) PCT Filed: Aug. 9, 2023
(86) PCT No.: PCT/US2023/029828

§ 371 Date: Feb. 10, 2025

Related U.S. Application Data
(60) Provisional application No. 63/396,459, filed on Aug. 9, 2022.

(51) Int. Cl. C07D 413/14; A61K 31/506; A61K 31/519; A61K 31/5377; C07D 239/42; C07D 403/12; C07D 403/14; C07D 413/12; C07D 487/04

(52) U.S. Cl. CPC C07D 413/14; A61K 31/506; A61K 31/519; A61K 31/5377; C07D 239/42; C07D 403/12; C07D 403/14; C07D 413/12; C07D 487/04

(57) ABSTRACT

The present disclosure provides compounds of Formula (I), Formula (II) and Formula (III) which are useful as inhibitors of ULK3 and methods of using the same to treat cancers, such as ULK-associated cancers, for example multiple myeloma and breast cancer.

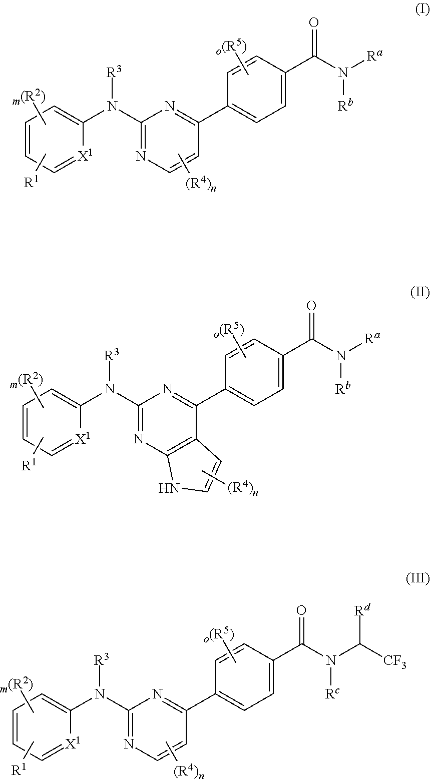

### CROSS-REFERENCE TO RELATED APPLICATIONS

This application claims the benefit of priority to U.S. Provisional Application No. 63/396,459 filed Aug. 9, 2022, the disclosure of which is incorporated herein by reference in its entirety.

### TECHNICAL FIELD

This disclosure relates to compounds for use in treating medical disorders, and more particularly to inhibitors of ULK3 which are useful in treating cancers, more particularly multiple myeloma.

### BACKGROUND

Autophagy is a highly dynamic multistep biological process of self-eating that removes unnecessary or dysfunctional components through a lysosome-dependent regulated mechanism. In some cancers, autophagy becomes a key survival mechanism for tumor cells under harsh conditions, such as hypoxia, nutrient limitation, and chemotherapy. Preclinical studies have shown that genetic or pharmacological inhibition of cytoprotective autophagy can overcome therapy resistance and promote tumor regression. The Unc-51 like kinase (ULK) complex is a key early initiator or autophagy.

There is a clear need for therapies for the treatment of cancers which block autophagy as a mechanism for tumor resistance and regression.

### SUMMARY

The present disclosure relates to compounds and compositions which are useful in treating medical disorders, in particular ULK3 inhibitors which are useful in treating cancers, such as ULK3-associated cancers, for example, multiple myeloma or breast cancer.

In one aspect, a compound is provided of Formula II or Formula III

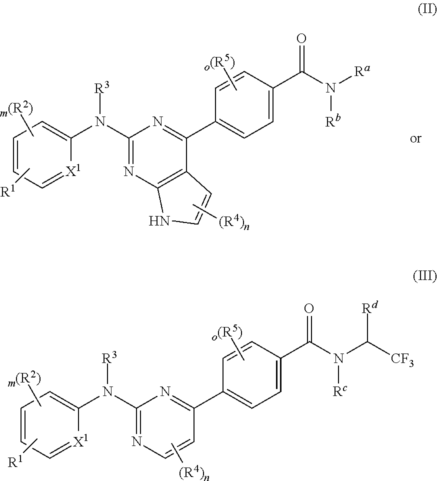

or
or a pharmaceutically acceptable salt or derivative thereof,
wherein all variables are as defined herein.

In another aspect, a pharmaceutical composition is provided comprising a compound described herein, or a pharmaceutically acceptable salt or derivative thereof, and a pharmaceutically acceptable carrier or excipient.

In a further aspect, a method is provided of treating a cancer in a subject in need thereof comprising administering to the subject a therapeutically effective amount of a compound described herein, or a pharmaceutically acceptable salt or derivative thereof, or a pharmaceutical composition of claim 17.

In yet a further aspect, a method is provided of treating a ULK3-associated cancer in a subject in need thereof comprising administering to the subject a therapeutically effective amount of a compound of Formula I

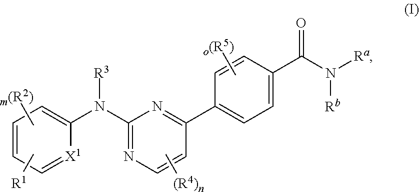

or a pharmaceutically acceptable salt or derivative thereof,
wherein all variables are as defined herein.

The details of one or more aspects of the disclosure are set forth in the description below. Other features, objects, and advantages of the disclosure will be apparent from the description and the claims.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain having the benefit of the teachings presented in the foregoing descriptions. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual aspects described and illustrated herein has discrete components and features which may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate.” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of this disclosure (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound or composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a cancer. The effect can be therapeutic in terms of a partial or complete cure of a disease, condition, symptom or adverse effect attributed to the disease, disorder, or condition. The term “treatment” as used herein can include any treatment of a disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this disclosure belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the disclosure and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In some aspects, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl) C₀-C₄alkyl, or —C₀-C₄ (C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one aspect, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one aspect, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one aspect, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one aspect, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 4, or in some aspects 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspect, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one aspect, the total number of S and O) atoms in the heteroaryl ring is not more than 2. In another aspect, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄-COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

### Compounds

In one aspect, a compound of Formula II or Formula III is provided

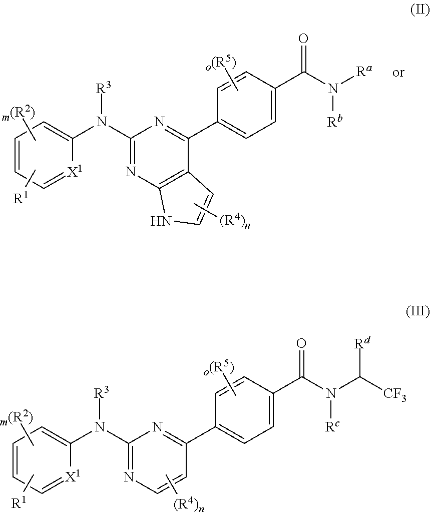

or
or a pharmaceutically acceptable salt or derivative thereof,
wherein:
m is 0, 1, 2, or 3;
n is 0, 1, or 2;
o is 0, 1, 2, 3, or 4;
R¹ is selected from —(C₀-C₃alkyl)(3- to 8-membered monocyclic or bicyclic heterocycle), R^{x}O—C(O)—(C₀-C₃ alkyl)-, and (R^{x}R^{y}N)—C(O)—(C₀-C₃ alkyl)-, each of which may be optionally substituted with one or more groups selected from Z as allowed by valency;
X¹ is selected from N or C(R²);
R², R⁴, and R⁵ are each independently selected at each occurrence from hydrogen, halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R*O—S(O)₂-(C₀-C₃ alkyl)-, (R^{x}R^{y}N) S(O)₂-(C₀-C₃ alkyl)-, R^{z}C(O)—O—(C₀-C₃ alkyl)-, R²C(O)—(RN)—(C₀-C₃ alkyl)-, R^{z}S(O)₂-O—(C₀-C₃ alkyl)-, R^{z}S(O)₂-(R^{x}N)—(C₀-C₃ alkyl)-, R^{z}C(O)—(C₀-C₆ alkyl)-, R^{z}S(O)—(C₀-C₃ alkyl)-, and R^{z}S(O)₂-(C₀-C₃ alkyl)-, each of which may be optionally substituted with one or more groups selected from Z as allowed by valency;
R³ is hydrogen or C₁-C₃ alkyl;
R^{a} and R^{b} are independently selected from hydrogen, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, and (5- to 10-membered monocyclic or bicyclic heteroaryl)-(C₀-C₃ alkyl)-, each of which may be optionally substituted with one or more groups selected from Z as allowed by valency;
or R^{a} and R^{b} are brought together with the nitrogen to which they are attached to form 3- to 8-membered monocyclic or bicyclic heterocycle optionally substituted with one or more groups selected from Z as allowed by valency;
R^{c} and R^{d} are independently selected from hydrogen and C₁-C₃ alkyl; or
R^{c} and R^{d} are brought together with the atoms to which they are attached to form a 3- to 8-membered monocyclic or bicyclic heterocycle optionally substituted with one or more groups selected from Z as allowable by valency;
Z is independently selected at each occurrence from hydrogen, halo, cyano, azido, oxo, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂-(C₀-C₃ alkyl)-, (R^{x}R^{y}N) S(O)₂-(C₀-C₃ alkyl)-, R²C(O)—O—(C₀-C₃ alkyl)-, R^{z}C(O)—(R^{x}N)—(C₀-C₃ alkyl)-, R^{z}S(O)₂-O—(C₀-C₃ alkyl)-, R^{z}S(O)₂-(R*N)—(C₀-C₃ alkyl)-, R^{z}C(O)—(C₀-C₆ alkyl)-, R^{z}S(O)—(C₀-C₃ alkyl)-, and R^{z}S(O)₂-(C₀-C₃ alkyl)-, each of which is optionally substituted with one or more groups selected from Y as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some aspects, R¹ is 5- to 6-membered monocyclic heterocycle. In some aspects, R¹ is —(CH₂)(5- to 6-membered monocyclic heterocycle). In some aspects, R¹ is R^{x}O—C(O)—(C₀-C₃ alkyl)-. In some aspects, R¹ is (R^{x}R^{y}N)—C(O)—(C₀-C₃ alkyl)-.

In some aspects, R¹ is selected from:

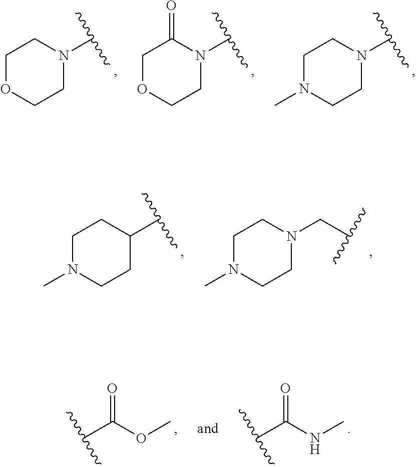

In some aspects, X¹ is N. In some aspects, X¹ is C(R²). In some aspects, X¹ is CH.

In some aspects,

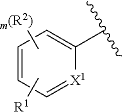

is selected from:

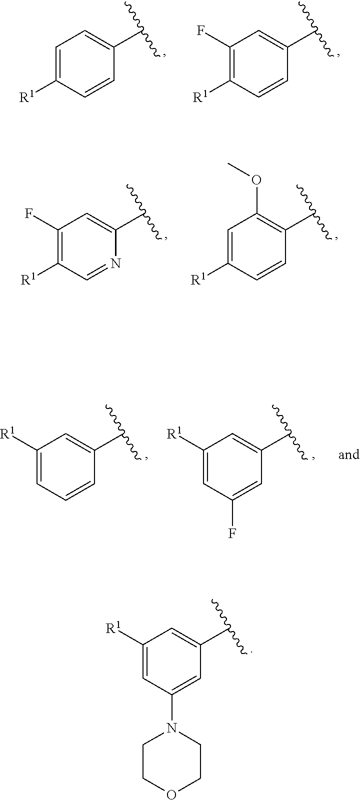

In some aspects, R³ is hydrogen. In some aspects, R³ is C₁-C₃ alkyl. In some aspects, R³ is methyl.

In some aspects,

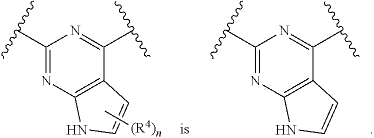

In some aspects,

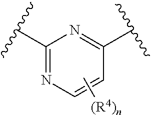

is selected from:

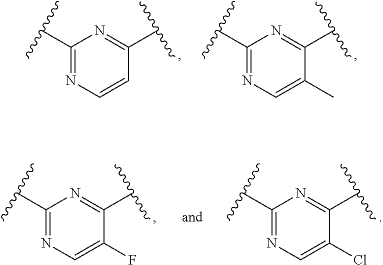

In some aspects,

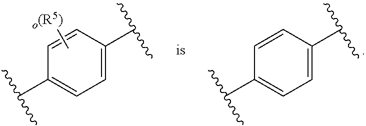

In some aspects, R^{a} is hydrogen and R^{b} is C₁-C₆ alkyl optionally substituted with 1 or 2 groups selected from Z.

In some aspects, R^{a} and R^{b} are brought together with the nitrogen to which they are attached to form a 3- to 6-membered monocyclic heterocycle optionally substituted with 1 or 2 groups selected from Z.

In some aspects,

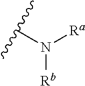

is selected from:

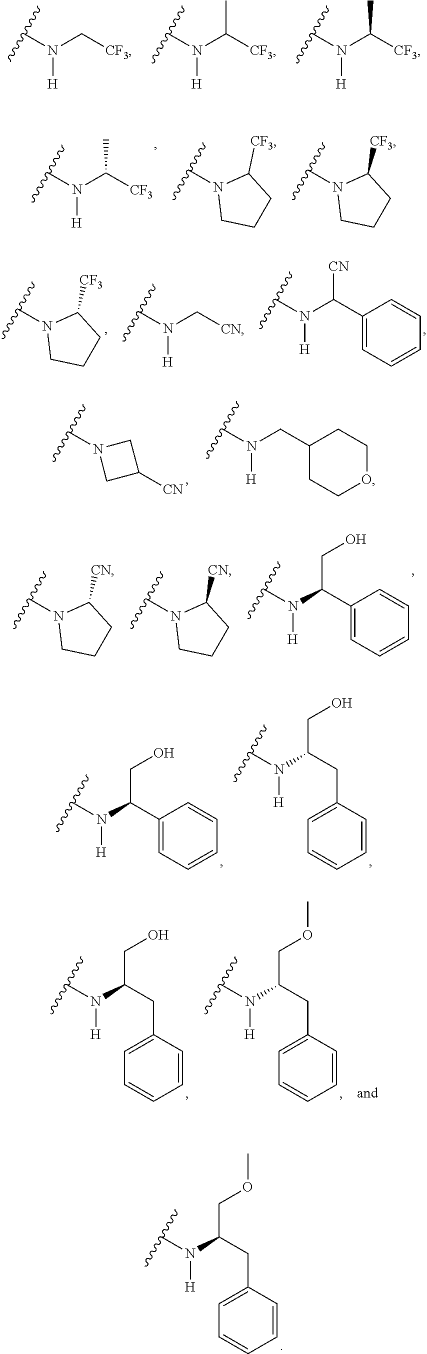

In some aspects,

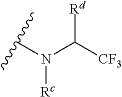

is selected from:

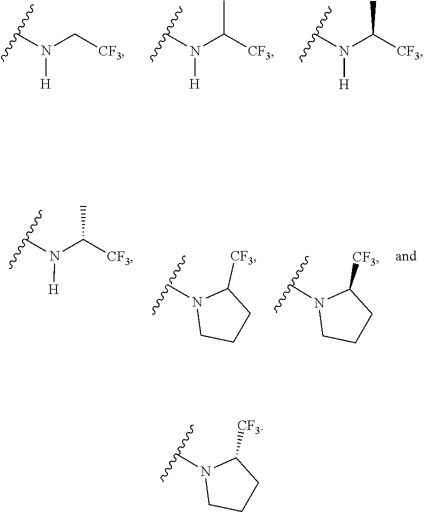

In another aspect, a compound of Formula I is provided

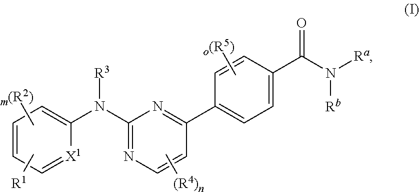

or a pharmaceutically acceptable salt or derivative thereof, wherein the compound of Formula I is not momelotinib, and wherein all variables are as defined herein.

In another aspect, a compound is provided selected from:

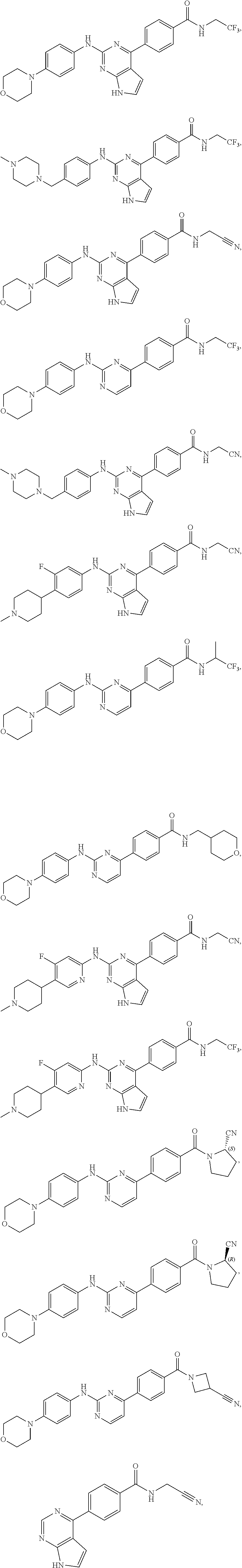

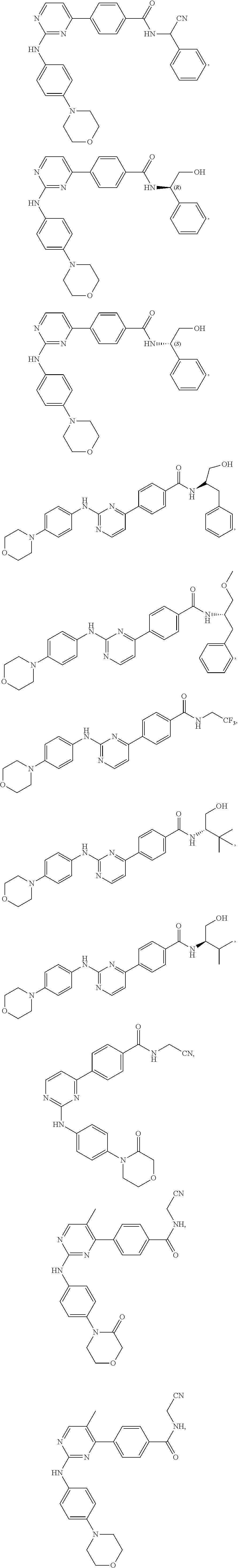

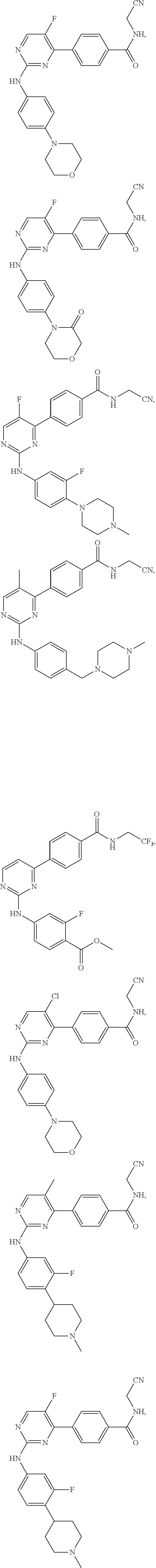

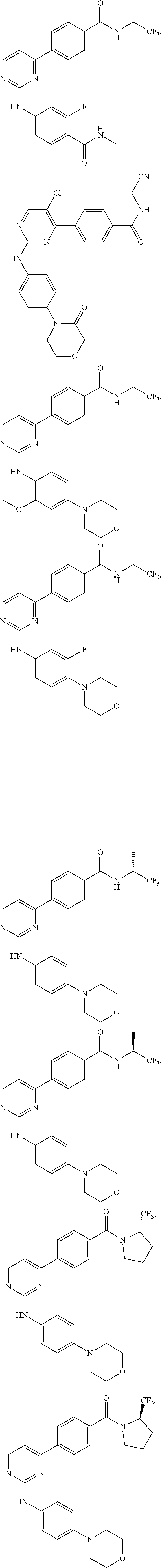

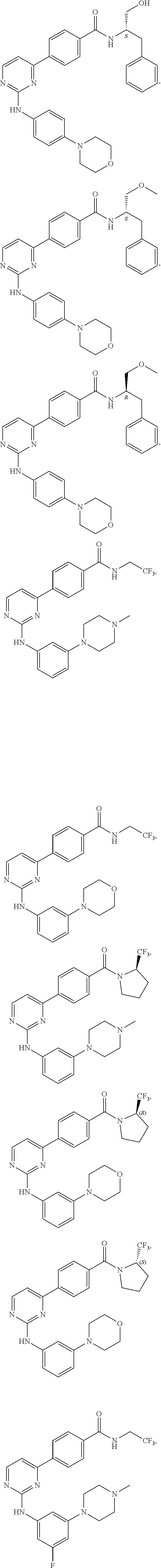

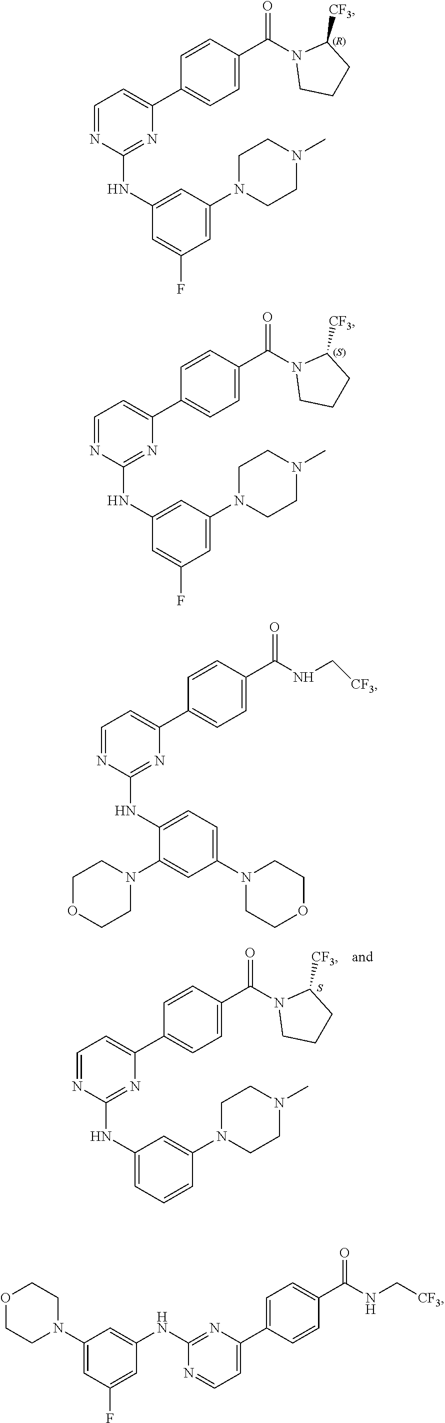

or a pharmaceutically acceptable salt or derivative thereof.

The present disclosure also includes compounds of Formula I and Formula II with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹¹C, ¹³C, ¹⁵N, ¹⁷O, ¹⁸O, ¹⁸F, ³¹P, ³²P, ³⁵S, ³⁶Cl, and ¹²⁵I, respectively. In one aspect, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this disclosure and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one aspect, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tmax, Cmax, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In some aspects, the isotope is 80, 85, 90, 95, or 99% or more enriched in an isotope at any location of interest. In some aspects, deuterium is 80, 85, 90, 95, or 99% enriched at a desired location. Unless otherwise stated, the enrichment at any point is above natural abundance, and in one aspect is enough to alter a detectable property of the compounds as a drug in a human.

The compounds of the present disclosure may form a solvate with solvents (including water). Therefore, in one aspect, this disclosure includes a solvated form of the active compound. The term “solvate” refers to a molecular complex of a compound of the present disclosure (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with this disclosure include those wherein the solvent of crystallization may be isotopically substituted, e.g., D₂O, d₆-acetone, or d₆-DMSO. A solvate can be in a liquid or solid form.

A “prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In some aspects, the prodrug renders the parent compound more lipophilic. In some aspects, a prodrug can be provided that has several prodrug moieties in a linear, branched, or cyclic manner. For example, non-limiting aspects include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some aspects, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO-(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether; and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some aspects, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment of a cancer in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some aspects, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether |Brij 30|), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In some aspects, the preservative is an anti-oxidant. In other aspects, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In some aspects, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In some aspects, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

### Methods of Use

The present disclosure also provides methods for treating or preventing cancer in a subject, comprising administering to the subject a therapeutically effective amount of a compound or composition disclosed herein. The methods can further comprise administering one or more additional therapeutic agents, for example anti-cancer agents or anti-inflammatory agents. Additionally, the method can further comprise administering a therapeutically effective amount of ionizing radiation to the subject.

Methods of killing a cancer or tumor cell are also provided comprising contacting the cancer or tumor cell with an effective amount of a compound or composition as described herein. In some aspects, the compounds can inhibit ULK3. The methods can further include administering one or more additional therapeutic agents or administering an effective amount of ionizing radiation.

The disclosed methods can optionally include identifying a patient who is or can be in need of treatment of an oncological disorder. The patient can be a human or other mammal, such as a primate (monkey, chimpanzee, ape, etc.), dog, cat, cow pig, or horse, or other animals having an oncological disorder. In some aspects, the subject can receive the therapeutic compositions prior to, during, or after surgical intervention to remove part or all of a tumor.

In one aspect, a method is provided for treating a cancer in a subject in need thereof, the method comprising administering to the subject a compound of Formula II or Formula III, or a pharmaceutically acceptable salt or derivative thereof. In some aspects, the cancer is a ULK-associated cancer.

In another aspect, a method is provided for treating a ULK3-associated cancer in a subject in need thereof comprising administering to the subject a compound of Formula I, or a pharmaceutically acceptable salt or derivative thereof.

As used herein, “ULK3-associated” or “associated with ULK3” refers to a disease or disorder, for example a cancer, associated with or having a dysregulation of a ULK3 gene, a ULK3 protein, or the expression or activity or level of any of the same. Thus, “ULK3-associated cancer” as used herein refers to a cancer having a dysregulation of a ULK3 gene, a ULK3 protein, or the expression or activity or level of any of the same. In some aspects, “ULK3-associated” refers to increased expression (e.g., increased levels) of a ULK3 protein in a cell due to aberrant cell signaling and/or dysregulated autocrine/paracrine signaling (as compared to a control non-cancerous cell).

The term “neoplasia” or “cancer” is used throughout this disclosure to refer to the pathological process that results in the formation and growth of a cancerous or malignant neoplasm, i.e., abnormal tissue (solid) or cells (non-solid) that grow by cellular proliferation, often more rapidly than normal and continues to grow after the stimuli that initiated the new growth cease. Malignant neoplasms show partial or complete lack of structural organization and functional coordination with the normal tissue and most invade surrounding tissues, can metastasize to several sites, are likely to recur after attempted removal and may cause the death of the patient unless adequately treated. As used herein, the term neoplasia is used to describe all cancerous disease states and embraces or encompasses the pathological process associated with malignant, hematogenous, ascitic and solid tumors. The cancers which may be treated by the compositions disclosed herein may comprise carcinomas, sarcomas, lymphomas, leukemias, germ cell tumors, or blastomas.

Carcinomas which may be treated by the compositions of the present disclosure include, but are not limited to, acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma,hyaline carcinoma, hypernephroid carcinoma, infantile embryonal carcinoma, carcinoma in situ, intraepidermal carcinoma, Krompecher's carcinoma, carcinoma, intraepithelial Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, and carcinoma vilosum.

Representative sarcomas which may be treated by the compositions of the present disclosure include, but are not limited to, liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma) skeletal and extra-skeletal, and chondrosarcoma.

The compositions of the present disclosure may be used in the treatment of a lymphoma. Lymphomas which may be treated include mature B cell neoplasms, mature T cell and natural killer (NK) cell neoplasms, precursor lymphoid neoplasms, Hodgkin lymphomas, and immunodeficiency-associated lymphoproliferative disorders. Representative mature B cell neoplasms include, but are not limited to, B-cell chronic lymphocytic leukemia/small cell lymphoma, B-cell prolymphocytic leukemia, lymphoplasmacytic lymphoma (such as Waldenström macroglobulinemia), splenic marginal zone lymphoma, hairy cell leukemia, plasma cell neoplasms (such as plasma cell myeloma/multiple myeloma, plasmacytoma, monoclonal immunoglobulin deposition diseases, and heavy chain diseases), extranodal marginal zone B cell lymphoma (MALT lymphoma), nodal marginal zone B cell lymphoma, follicular lymphoma, primary cutaneous follicular center lymphoma, mantle cell lymphoma, diffuse large B cell lymphoma, diffuse large B-cell lymphoma associated with chronic inflammation, Epstein-Barr virus-positive DLBCL of the elderly, lyphomatoid granulomatosis, primary mediastinal (thymic) large B-cell lymphoma, intravascular large B-cell lymphoma, ALK+ large B-cell lymphoma, plasmablastic lymphoma, primary effusion lymphoma, large B-cell lymphoma arising in HHV8-associated multicentric Castleman's disease, and Burkitt lymphoma/leukemia. Representative mature T cell and NK cell neoplasms include, but are not limited to, T-cell prolymphocytic leukemia, T-cell large granular lymphocyte leukemia, aggressive NK cell leukemia, adult T-cell leukemia/lymphoma, extranodal NK/T-cell lymphoma, nasal type, enteropathy-associated T-cell lymphoma, hepatosplenic T-cell lymphoma, blastic NK cell lymphoma, lycosis fungoides/Sezary syndrome, primary cutaneous CD30-positive T cell lymphoproliferative disorders (such as primary cutaneous anaplastic large cell lymphoma and lymphomatoid papulosis), peripheral T-cell lymphoma not otherwise specified, angioimmunoblastic T cell lymphoma, and anaplastic large cell lymphoma. Representative precursor lymphoid neoplasms include B-lymphoblastic leukemia/lymphoma not otherwise specified, B-lymphoblastic leukemia/lymphoma with recurrent genetic abnormalities, or T-lymphoblastic leukemia/lymphoma. Representative Hodgkin lymphomas include classical Hodgkin lymphomas, mixed cellularity Hodgkin lymphoma, lymphocyte-rich Hodgkin lymphoma, and nodular lymphocyte-predominant Hodgkin lymphoma.

The compositions of the present disclosure may be used in the treatment of a Leukemia. Representative examples of leukemias include, but are not limited to, acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia, adult T-cell leukemia, clonal eosinophilias, and transient myeloproliferative disease.

The compositions of the present disclosure may be used in the treatment of a germ cell tumor, for example germinomatous (such as germinoma, dysgerminoma, and seminoma), non germinomatous (such as embryonal carcinoma, endodermal sinus tumor, choriocarcinoma, teratoma, polyembryoma, and gonadoblastoma) and mixed tumors.

The compositions of the present disclosure may be used in the treatment of blastomas, for example hepatoblastoma, medulloblastoma, nephroblastoma, neuroblastoma, pancreatoblastoma, pleuropulmonary blastoma, retinoblastoma, and glioblastoma multiforme.

Representative cancers which may be treated include, but are not limited to: bone and muscle sarcomas such as chondrosarcoma, Ewing's sarcoma, malignant fibrous histiocytoma of bone/osteosarcoma, osteosarcoma, rhabdomyosarcoma, and heart cancer; brain and nervous system cancers such as astrocytoma, brainstem glioma, pilocytic astrocytoma, ependymoma, primitive neuroectodermal tumor, cerebellar astrocytoma, cerebral astrocytoma, glioma, medulloblastoma, neuroblastoma, oligodendroglioma, pineal astrocytoma, pituitary adenoma, and visual pathway and hypothalamic glioma; breast cancers including invasive lobular carcinoma, tubular carcinoma, invasive cribriform carcinoma, medullary carcinoma, male breast cancer, Phyllodes tumor, and inflammatory breast cancer; endocrine system cancers such as adrenocortical carcinoma, islet cell carcinoma, multiple endocrine neoplasia syndrome, parathyroid cancer, phemochromocytoma, thyroid cancer, and Merkel cell carcinoma; eye cancers including uveal melanoma and retinoblastoma; gastrointestinal cancers such as anal cancer, appendix cancer, cholangiocarcinoma, gastrointestinal carcinoid tumors, colon cancer, extrahepatic bile duct cancer, gallbladder cancer, gastric cancer, gastrointestinal stromal tumor, hepatocellular cancer, pancreatic cancer, and rectal cancer; genitourinary and gynecologic cancers such as bladder cancer, cervical cancer, endometrial cancer, extragonadal germ cell tumor, ovarian cancer, ovarian epithelial cancer, ovarian germ cell tumor, penile cancer, renal cell carcinoma, renal pelvis and ureter transitional cell cancer, prostate cancer, testicular cancer, gestational trophoblastic tumor, urethral cancer, uterine sarcoma, vaginal cancer, vulvar cancer, and Wilms tumor; head and neck cancers such as esophageal cancer, head and neck cancer, nasopharyngeal carcinoma, oral cancer, oropharyngeal cancer, paranasal sinus and nasal cavity cancer, pharyngeal cancer, salivary gland cancer, and hypopharyngeal cancer; hematopoietic cancers such as acute biphenotypic leukemia, acute eosinophilic leukemia, acute lymphoblastic leukemia, acute myeloid leukemia, acute myeloid dendritic cell leukemia, AIDS-related lymphoma, anaplastic large cell lymphoma, angioimmunoblastic T-cell lymphoma, B-cell prolymphocytic leukemia, Burkitt's lymphoma, chronic lymphocytic leukemia, chronic myelogenous leukemia, cutaneous T-cell lymphoma, diffuse large B-cell lymphoma, follicular lymphoma, hairy cell leukemia, hepatosplenic T-cell lymphoma, Hodgkin's lymphoma, hairy cell leukemia, intravascular large B-cell lymphoma, large granular lymphocytic leukemia, lymphoplasmacytic lymphoma, lymphomatoid granulomatosis, mantle cell lymphoma, marginal zone B-cell lymphoma, Mast cell leukemia, mediastinal large B cell lymphoma, multiple myeloma/plasma cell neoplasm, myelodysplastic syndroms, mucosa-associated lymphoid tissue lymphoma, mycosis fungoides, nodal marginal zone B cell lymphoma, non-Hodgkin lymphoma, precursor B lymphoblastic leukemia, primary central nervous system lymphoma, primary cutaneous follicular lymphoma, primary cutaneous immunocytoma, primary effusion lymphoma, plasmablastic lymphoma, Sezary syndrome, splenic marginal zone lymphoma, and T-cell prolymphocytic leukemia; skin cancers such as basal cell carcinoma, squamous cell carcinoma, skin adnexal tumors (such as sebaceous carcinoma), melanoma, Merkel cell carcinoma, sarcomas of primary cutaneous origin (such as dermatofibrosarcoma protuberans), and lymphomas of primary cutaneous origin (such as mycosis fungoides); thoracic and respiratory cancers such as bronchial adenomas/carcinoids, small cell lung cancer, mesothelioma, non-small cell lung cancer, pleuropulmonary blastoma, laryngeal cancer, and thymoma or thymic carcinoma; HIV/AIDS-related cancers such as Kaposi sarcoma; epithelioid hemangioendothelioma; desmoplastic small round cell tumor; and liposarcoma.

In particular aspects, the cancer is multiple myeloma. In other particular aspects, the cancer is breast cancer.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can also be systemically administered, such as intravenously or orally, optionally in combination with a pharmaceutically acceptable carrier such as an inert diluent, or an assimilable edible carrier for oral delivery. In addition, the active compound can be incorporated into sustained release preparations and/or devices.

For the treatment of oncological disorder, compounds, agents, and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds, agents, and compositions disclosed herein can be used in methods of treating cancer wherein the patient is to be treated or is or has been treated with mitotic inhibitors such as taxol or vinblastine, alkylating agents such as cyclophosphamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, imatinid or trastuzumab. These other substances or radiation treatments can be given at the same time as or at different times from the compounds disclosed herein. Examples of other suitable chemotherapeutic agents include, but are not limited to, altretamine, bleomycin, bortezomib, busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib, gemcitabine, hydroxyurea, idarubicin, ifosfamide, imatinib, irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uraxil, temozolomide, thiotepa, tioguanine/thioguanine, topotexan, treosulfan, vinblastine, vincristine, vindesine, and vinorelbine. Examples of suitable immunotherapeutic agents include, but are not limited to, alemtuzumab, cetuximab, gemtuzumab, iodine 131 tositumomab, rituximab, and trastuzumab. Cytotoxic agents include, for example, radioactive isotopes and toxins of bacterial, fungal, plant, or animal origin. Also disclosed are methods of treating an oncological disorder comprising administering an effective amount of a compound described herein prior to, subsequent to, and/or in combination with administration of a chemotherapeutic agent, an immunotherapeutic agent, a radiotherapeutic agent, or radiotherapy.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some aspects, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary and can be administered in one or more dose administrations daily, for one or several days.

### Kits

Kits for practicing the methods described herein are further provided. By “kit” is intended any manufacture (e.g., a package or a container) comprising at least one reagent, e.g., any one of the compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

To provide for the administration of such dosages for the desired therapeutic treatment, in some aspects, pharmaceutical compositions disclosed herein can comprise between 0.1% and 45%, and especially, 1 and 15%, by weight of the total of one or more of the compounds based on the weight of the total composition including carriers and/or diluents. Illustratively, dosage levels of the administered active ingredients can be: intravenous 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasally, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one aspect, a kit includes one or moe other components, adjuncts, or adjuvants as described herein. In another aspect, a kit includes one or more anti-cancer agents, such as those agents described herein. In one aspect, a kit includes instructions or packaging materials that describe how to administer a compound or composition of the kit. Containers of the kit can be of any suitable material, e.g., glass, plastic, metal, etc., and of any suitable size, shape, or configuration. In one aspect, a compound and/or agent disclosed herein is provided in the kit as a solid, such as a tablet, pill, or powder form. In another aspect, a compound and/or agent disclosed herein is provided in the kit as a liquid or solution. In one aspect, the kit comprises an ampoule or syringe containing a compound and/or agent disclosed herein in liquid or solution form.

A number of aspects of the disclosure have been described. Nevertheless, it will be understood that various modifications may be made without departing from the spirit and scope of this disclosure. Accordingly, other aspects are within the scope of the following claims.

By way of non-limiting illustration, examples of some embodiments of the present disclosure are given below.

### EXAMPLES

The following examples are put forth so as to provide those of ordinary skill in the art with a complete disclosure and description of how the compounds, compositions, and/or methods claimed herein are made and evaluated and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Synthetic Examples

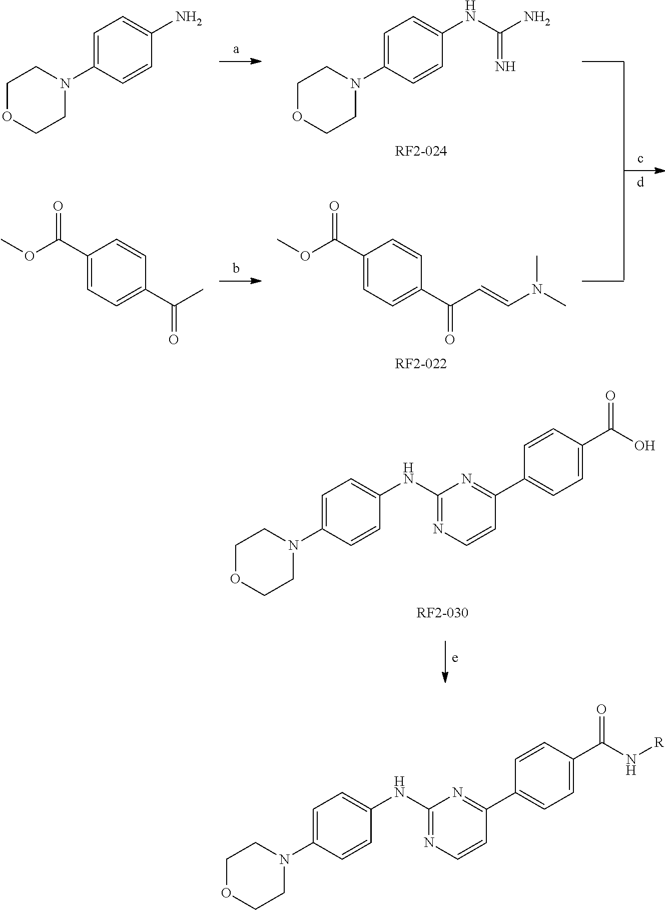

Reagents and Conditions: (a) NH₂CN, conc HCl, EtOH, reflux, 6 h, 59%; (b) DMF-DMA, 85° C., 20 h, 78%, (c) NaOH, n-BuOH, reflux, 16 h; (d) LiOH, acetone/H₂O, reflux, 2 h, 27% (overall, two steps); (e) amine, EDCl, HOBt, TEA, DMF, r.t., 17 h.

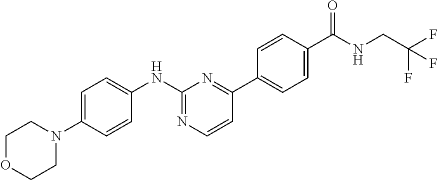 [SMILES: O=C(NCC(F)(F)F)c1ccc(-c2ccnc(Nc3ccc(N4CCOCC4)cc3)n2)cc1]

4-(2-((4-Morpholinophenyl)amino)pyrimidin-4-yl)-N-(3,3,3-trifluoropropyl)benzamide (BH1-058): 4-(2-((4-Morpholinophenyl)amino)pyrimidin-4-yl)benzoic acid (RF2-030 prepared according to a reported method) 1 (100 mg, 0.266 mmol), 3,3,3-trifluoropropan-1-amine hydrochloride (50.0 mg, 0.333 mmol), 1-(3-dimethylaminopropyl)-3-ethylcarbodiimide hydrochloride (76.0 mg, 0.399 mmol), and 1-hydroxybenzotriazole hydrate (54.0 mg, 0.399 mmol) were dissolved in dry dimethylformamide (2.00 mL, 0.133 M) and round bottom flask was degassed with argon for 10 minutes. Dry triethylamine (67.0 mg, 0.0930 mL, 0.665 mmol) was added, and the reaction was allowed to stir at room temperature under argon for 12 hours. The reaction mixture was diluted with ethyl acetate (30 mL) and washed successively with water (2×20 mL), saturated ammonium chloride (20 mL), and brine (20 mL). The organic layer was dried with sodium sulfate, filtered, and concentrated under reduced pressure to yield BH1-058 (102 mg, 82%) as a yellow powder. ¹H NMR (500 MHz, DMSO-d6) δ 9.49 (s, 1H), 8.82 (t, J=5.6 Hz, 1H), 8.54 (d, J=5.2 Hz, 1H), 8.25 (d, J=8.5 Hz, 2H), 7.99 (d, J=8.5 Hz, 2H), 7.68 (d, J=9.0 Hz, 2H), 7.40 (d, J=5.1 Hz, 1H), 6.94 (d, J=9.1 Hz, 2H), 3.82-3.72 (m, 4H), 3.58-3.49 (m, 2H), 3.10-3.03 (m, 4H), 2.73-2.55 (m, 2H); ¹⁹F NMR (471 MHz, DMSO-d6) δ −63.82 (t, J=11.4 Hz); HPLC-MS (ESI+) m/z 472.2 (M+H)⁺.

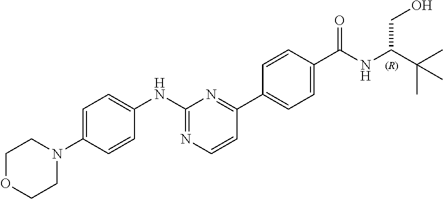

(S)—N-(1-Hydroxy-3,3-dimethylbutan-2-yl)-4-(2-((4-morpholinophenyl)amino)pyrimidin-4-yl)benzamide (BH1-062): 4-(2-((4-Morpholinophenyl)amino)pyrimidin-4-yl)benzoic acid (RF2-030) (50.0 mg, 0.133 mmol), (S)-2-amino-3,3-dimethylbutan-1-ol (20.0 mg, 0.166 mmol), 1-(3-dimethylaminopropyl)-3-ethylcarbodiimide hydrochloride (38.0 mg, 0.200 mmol), and 1-hydroxybenzotriazole hydrate (27.0 mg, 0.200 mmol) were dissolved in dry dimethylformamide (1.00 mL, 0.133 M) and round bottom flask was degassed with argon for 10 minutes. Dry triethylamine (34.0 mg, 0.0360 mL, 0.333 mmol) was added, and the reaction was allowed to stir at room temperature under argon for 17 hours. The reaction mixture was diluted with ethyl acetate (50 mL) and washed successively with water (2×20 mL), saturated ammonium chloride (20 mL), and brine (20 mL). The organic layer was dried with sodium sulfate, filtered, and concentrated under reduced pressure to yield BH1-062 (53 mg, 84%) as a yellow powder. ¹H NMR (500 MHz, DMSO-d6) δ 9.54 (s, 1H), 8.59 (d, J=5.1 Hz, 1H), 8.29 (d, J=8.4 Hz, 2H), 8.08-8.04 (m, 3H), 7.74 (d, J=9.0 Hz, 2H), 7.45 (d, J=5.2 Hz, 1H), 7.00 (d, J=9.1 Hz, 2H), 4.52 (t, J=5.6 Hz, 1H), 3.99 (td, J=9.1, 3.6 Hz, 1H), 3.81 (t, J=5.0 Hz, 4H), 3.75 (ddd, J=11.1, 5.2, 3.6 Hz, 1H), 3.56 (ddd, J=11.1, 8.9, 6.1 Hz, 1H), 3.11 (t, J=5.0 Hz, 4H), 0.99 (s, 9H); HPLC-MS (ESI+) m/z 476.3 (M+H)⁺; HRMS (TOF) m/z 476.2658 (M+H)⁺.

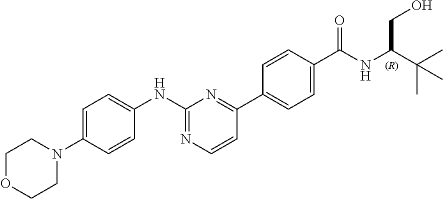

(R)—N-(1-Hydroxy-3-methylbutan-2-yl)-4-(2-((4-morpholinophenyl)amino)pyrimidin-4-yl)benzamide (BH1-063): 4-(2-((4-Morpholinophenyl)amino)pyrimidin-4-yl)benzoic acid (RF2-030) (38.0 mg, 0.101 mmol), (R)-2-amino-3-methylbutan-1-ol (13.0 mg, 0.126 mmol), 1-(3-dimethylaminopropyl)-3-ethylcarbodiimide hydrochloride (29.0 mg, 0.152 mmol), and 1-hydroxybenzotriazole hydrate (21.0 mg, 0.152 mmol) were dissolved in dry dimethylformamide (0.800 mL, 0.126 M) and round bottom flask was degassed with argon for 10 minutes. Dry triethylamine (26.0 mg, 0.0360 mL, 0.253 mmol) was added, and the reaction was allowed to stir at room temperature under argon for 17 hours. The reaction mixture was diluted with ethyl acetate (50 mL) and washed successively with water (2×20 mL), saturated ammonium chloride (20 mL), and brine (20 mL). The organic layer was dried with sodium sulfate, filtered, and concentrated under reduced pressure to yield BH1-063 (32 mg, 70%) as a yellow powder. ¹H NMR (500 MHz, DMSO-d6) δ 9.49 (s, 1H), 8.53 (d, J=5.2 Hz, 1H), 8.23 (d, J=8.3 Hz, 2H), 8.10 (d, J=8.9 Hz, 1H), 8.02 (d, J=8.3 Hz, 2H), 7.69 (d, J=9.0 Hz, 2H), 7.40 (d, J=5.2 Hz, 1H), 6.94 (d, J=9.1 Hz, 2H), 4.60 (t, J=5.6 Hz, 1H), 3.93-3.80 (m, 1H), 3.75 (t, J=4.8 Hz, 4H), 3.54 (hept, J=5.9 Hz, 2H), 3.06 (t, J=4.8 Hz, 4H), 1.96 (tt, J=13.9, 6.7 Hz, 1H), 0.92 (dd, J=13.1, 6.8 Hz, 6H); HPLC-MS (ESI+) m/z 462.3 (M+H)⁺.

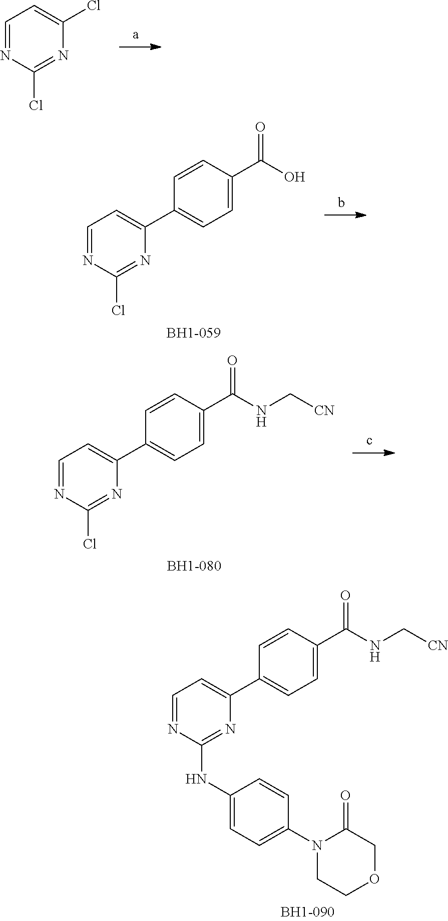 [SMILES: Clc1ccnc(Cl)n1 ; O=C(O)c1ccc(-c2ccnc(Cl)n2)cc1 ; N#CCNC(=O)c1ccc(-c2ccnc(Cl)n2)cc1 ; N#CCNC(=O)c1ccc(-c2ccnc(Nc3ccc(N4CCOCC4=O)cc3)n2)cc1]

Reagents and Conditions: (a) 4-boronobenzoic acid, Pd(PPh₃)₂Cl₂, aq. K₂CO₃, 1,4-dioxane, 70° C., 2 h, 76%; (b) 2-aminoacetonitrile hydrochloride, 1-ethyl-3-(3-dimethylaminopropyl)carbodiimide), hydroxybenzotriazole monohydrate, triethylamine, DMF, r.t., 20 h, 23%; (c) 4-(4-aminophenyl) morpholin-3-one, K₂CO₃, XPhos, Pd₂(dba)₃, 1,4-dioxane, 100° C., 4 h, 26%.

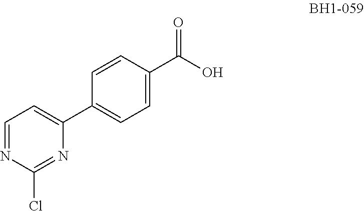 [SMILES: O=C(O)c1ccc(-c2ccnc(Cl)n2)cc1]

4-(2-Chloropyrimidin-4-yl)benzoic acid (BH1-059): 2,4-Dichloropyrimidine (500 mg, 3.38 mmol) and 4-boronobenzoic acid (841 mg, 5.07 mmol) were dissolved in aqueous 2M potassium carbonate (13.6 mL) and dioxane (20.4 mL) and round bottom flask was purged with argon 10 minutes. Bis(triphenylphosphine) palladium (II) dichloride (118 mg, 0.169 mmol) was added under argon and reaction was allowed to stir at 90° C. for 2 hours. The reaction was cooled to room temperature, dioxane evaporated off, and solution was acidified to ˜ pH 3. The aqueous solution was extracted with ethyl acetate (2×200 mL) and the resulting organic layer was washed with brine (50 mL). The organic layer was dried with sodium sulfate, filtered, and concentrated under reduced pressure. The residue was triturated with 80:20 ethyl acetate: hexanes three times to yield BH1-059 (603 mg, 76%) as a white powder. ¹H NMR (500 MHz, DMSO-d6) δ 13.21 (s, 1H), 8.90 (d, J=5.3 Hz, 1H), 8.31 (d, J=8.5 Hz, 2H), 8.23 (d, J=5.3 Hz, 1H), 8.11 (d, J=8.4 Hz, 2H).

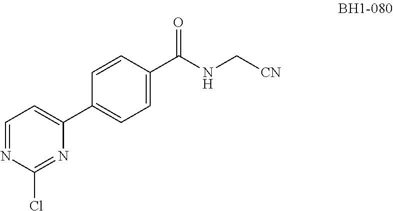 [SMILES: N#CCNC(=O)c1ccc(-c2ccnc(Cl)n2)cc1]

4-(2-Chloropyrimidin-4-yl)-N-(cyanomethyl)benzamide (BH1-080): 4-(2-Chloropyrimidin-4-yl)benzoic acid (BH1-059) (500 mg, 2.13 mmol), 2-aminoacetonitrile hydrochloride (246 mg. 2.66 mmol), 1-(3-dimethylaminopropyl)-3-ethylcarbodiimide hydrochloride (613 mg, 3.20 mmol), and hydroxybenzotriazole (433 mg, 3.20 mmol) was dissolved in dry dimethylformamide (15.8 mL, 0.135 M) and round bottom flask was degassed with argon 10 minutes. Dry triethylamine (539 mg, 0.742 mL, 5.33 mmol) was added and the reaction was allowed to stir under argon for 20 hours. The reaction was diluted with ethyl acetate (100 mL) and washed successively with water (3×50 mL), saturated ammonium chloride (50 mL), and brine (3×50 mL). The organic layer was dried with sodium sulfate, filtered, and concentrated under reduced pressure. The crude residue was subjected to flash column chromatography using a 50-80% ethyl acetate in hexanes gradient to yield BH1-080 (135 mg, 23%) as a white solid. ¹H NMR (500 MHz, DMSO-d6) δ 9.40 (t, J=5.5 Hz, 1H), 8.89 (d, J=5.3 Hz, 1H), 8.33 (d, J=8.5 Hz, 2H), 8.25 (d, J=5.2 Hz, 1H), 8.05 (d, J=8.5 Hz, 2H), 4.36 (d, J=5.4 Hz, 2H); HPLC-MS (ESI+) m/z 273.1 (M+H)⁺.

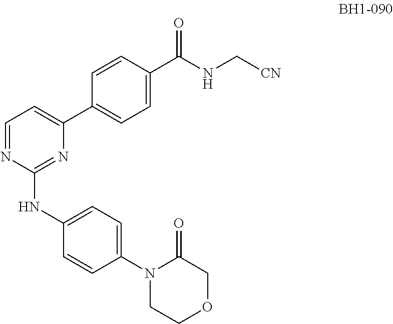 [SMILES: N#CCNC(=O)c1ccc(-c2ccnc(Nc3ccc(N4CCOCC4=O)cc3)n2)cc1]

N-(Cyanomethyl)-4-(2-((4-(3-oxomorpholino) phenyl)amino)pyrimidin-4-yl)benzamide (BH1-090): 4-(2-Chloropyrimidin-4-yl)-N-(cyanomethyl)benzamide (BH1-080) (50.0 mg, 0.183 mmol), 4-(4-aminophenyl) morpholin-3-one (35.0 mg, 0.183 mmol), potassium carbonate (51.0 mg, 0.366 mmol), and Xphos (18.0 mg, 0.0369 mmol) was dissolved in dry dioxane (1.40 mL, 0.131 M). The pressure vessel was flushed with argon for 10 minutes and tris(dibenzylideneacetone) dipalladium (0) (17.0 mg, 0.0183 mmol) was added under argon. The vessel was sealed and allowed to stir at 100° C. for 4 hours. The reaction was diluted with ethyl acetate (50 mL) and washed successively with water (3×30 mL) and brine (30 mL). The organic layer was dried with sodium sulfate, filtered and concentrated under reduced pressure. The crude residue was subjected to flash column chromatography with a 0-5% methanol and dichloromethane eluent to yield BH1-090 (20.0 mg, 26%) as a white solid. ¹H NMR (500 MHz, DMSO-d6) δ 9.84 (s, 1H), 9.35 (t, J=5.5 Hz, 1H), 8.62 (d, J=5.1 Hz, 1H), 8.30 (d, J=8.3 Hz, 2H), 8.04 (d, J=8.4 Hz, 2H), 7.85 (d, J=8.9 Hz, 2H), 7.51 (d, J=5.2 Hz, 1H), 7.32 (d, J=8.8 Hz, 2H), 4.36 (d, J=5.4 Hz, 2H), 4.19 (s, 2H), 3.98 (dd, J=6.0, 4.1 Hz, 2H), 3.72 (dd, J=5.9, 4.2 Hz, 2H); HPLC-MS (ESI+) m/z 429.3 (M+H)⁺;)⁺; HRMS (TOF) m/z 429.1663 (M+H)⁺.

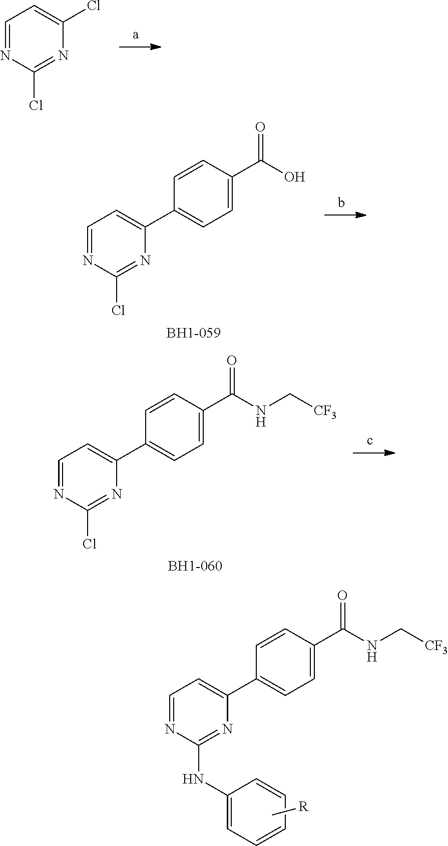

Reagents and Conditions: (a) 4-boronobenzoic acid, Pd(PPh₃)₂Cl₂, aq. K₂CO₃, 1,4-dioxane, 70° C., 2 h, 76%; (b) HATU, 2,2,2-trifluoroethan-1-amine hydrochloride, DMF, rt, 1.5 h, 43%; (c) aniline, K₂CO₃, XPhos, Pd₂(dba)₃, 1,4-dioxane, 100° C., 4 h.

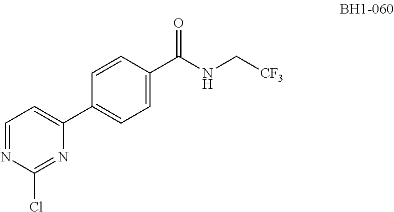 [SMILES: O=C(NCC(F)(F)F)c1ccc(-c2ccnc(Cl)n2)cc1]

4-(2-Chloropyrimidin-4-yl)-N-(2,2,2-trifluoroethyl)benzamide (BH1-060): 4-(2-Chloropyrimidin-4-yl)benzoic acid (BH1-059) (587 mg, 2.50 mmol), 2,2,2-trifluoroethan-1-amine hydrochloride (373 mg, 2.75 mmol), and 1-[bis(dimethylamino)methylene]-1H-1,2,3-triazolo[4,5-b]pyridinium 3-oxide hexafluorophosphate (1.05 g, 2.75 mmol) was dissolved in dry dimethylformamide (18.0 mL, 0.139 M) and round bottom flask was degassed with argon 10 minutes. Dry N-ethyldiisopropylamine (679 mg, 0.92 mL, 5.254 mmol) was added and the reaction was allowed to stir under argon for 1.5 hours. The reaction was diluted with ethyl acetate (300 mL) and washed successively with water (2×100 mL), saturated ammonium chloride (50 mL), and brine (50 mL). The organic layer was dried with sodium sulfate, filtered, and concentrated under reduced pressure. The residue was subjected to column chromatography using a 20 to 80% ethyl acetate in hexanes gradient to yield BH1-060 (338 mg, 43%) as a white solid. ¹H NMR (500 MHz, DMSO-d6) δ 9.29 (t, J=6.2, 1H), 8.89 (d, J=5.3 Hz, 1H), 8.32 (d, J=8.4 Hz, 2H), 8.25 (d, J=5.3 Hz, 21H), 8.07 (d, J=8.4 Hz, 2H), 4.18-4.06 (m, 2H); ¹⁹F NMR (471 MHz, DMSO-d6) δ −70.33 (t, J=9.7 Hz); HPLC-MS (ESI+) m/z 316.1 (M+H)⁺.

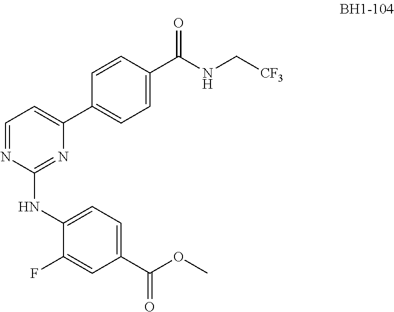 [SMILES: COC(=O)c1ccc(Nc2nccc(-c3ccc(C(=O)NCC(F)(F)F)cc3)n2)c(F)c1]

Methyl 2-fluoro-4-((4-(4-((2,2,2-trifluoroethyl)carbamoyl)phenyl)pyrimidin-2-yl) amino)benzoate (BH1-104): This was prepared in the same way as BH1-090, from BH1-060 (50 mg, 0.158 mmol) and methyl 4-amino-2-fluorobenzoate (24 mg, 0.142 mmol) with a 16 hour reaction time. The crude material subjected to flash column chromatography with a 20-70% ethyl acetate and hexanes eluent to yield BH1-104 (22.0 mg, 35%) as a tan solid. ¹H NMR (500 MHz, DMSO-d6) δ 10.44 (s, 1H), 9.25 (t, J=6.3 Hz, 1H), 8.73 (d, J=5.3 Hz, 1H), 8.31 (d, J=8.6 Hz, 2H), 8.08 (d, J=8.6 Hz, 2H), 8.00 (dd, J=14.5, 2.1 Hz, 1H), 7.87 (t, J=8.7 Hz, 1H), 7.73-7.64 (m, 2H), 4.13 (qd, J=9.7, 6.3 Hz, 2H), 3.82 (s, 3H); 19F NMR (471 MHz, DMSO-d6) δ −70.35 (t, J=9.9 Hz), —107.77 (dd, J=14.5, 8.7 Hz); HPLC-MS (ESI+) m/z 449.0 (M+H)⁺;)⁺; HRMS (TOF) m/z 449.1229 (M+H)⁺.

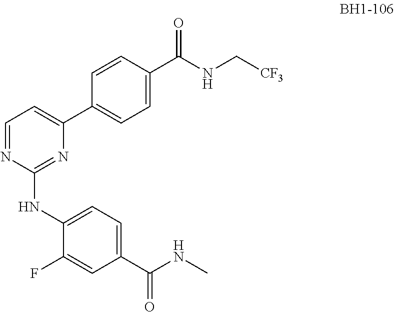 [SMILES: CNC(=O)c1ccc(Nc2nccc(-c3ccc(C(=O)NCC(F)(F)F)cc3)n2)c(F)c1]

2-Fluoro-N-methyl-4-((4-(4-((2,2,2-trifluoroethyl)carbamoyl)phenyl)pyrimidin-2-yl) amino)benzamide (BH1-106): This was prepared in the same way as BH1-090 from BH1-070 (50 mg, 0.158 mmol) and 4-amino-3-fluoro-N-methylbenzamide (24 mg, 0.142 mmol) with a 16 hour reaction time The crude material subjected to flash column chromatography with a 20-60% ethyl acetate and hexanes eluent to yield BH1-106 (27.0 mg, 45%) as a white solid. ¹H NMR (500 MHz, DMSO-d6) δ 10.25 (s, 1H), 9.25 (1, J=6.3 Hz, 1H), 8.70 (d, J=5.2 Hz, 1H), 8.30 (d, J=8.2 Hz, 2H), 8.07 (d, J=8.1 Hz, 2H), 8.01-7.89 (m, 2H), 7.74-7.55 (m, 3H), 4.13 (dt, J=16.1, 8.0 Hz, 2H), 2.78 (d, J=4.5 Hz, 3H); 19F NMR (471 MHz, DMSO-d6) δ −70.35 (t, J=9.7 Hz), —112.01 (ddd, J=13.8, 8.4, 4.2 Hz); HPLC-MS (ESI+) m/z 448.2 (M+H)⁺;)⁺; HRMS (TOF) m/z 448.1385 (M+H)⁺.

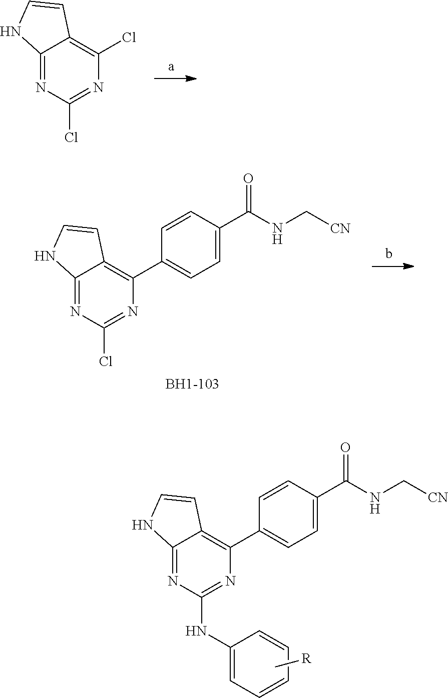

Reagents and Conditions: (a) (4-((cyanomethyl)carbamoyl)phenyl)boronic acid, Pd(PPh₃)₄, aq. Na₂CO₃, 1,4-dioxane, 150° C., 15 h, 63%; (b) aniline, K₂CO₃, XPhos, Pd₂(dba)₃, 1,4-dioxane, 100° C., 16 h.

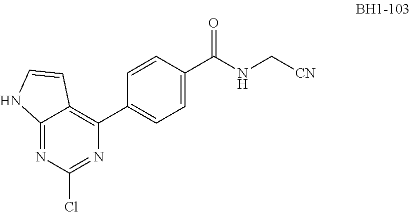 [SMILES: N#CCNC(=O)c1ccc(-c2nc(Cl)nc3[nH]ccc23)cc1]

4-(2-Chloro-7H-pyrrolo[2,3-d]pyrimidin-4-yl)-N-(cyanomethyl)benzamide (BH1-103): 2,4-Dichloro-7H-pyrrolo[2,3-d]pyrimidine (1.00 g, 5.12 mmol), (4-((cyanomethyl)carbamoyl)phenyl)boronic acid (1.30 g, 6.34 mmol), and sodium carbonate (1.69 g, 15.96 mmol) was dissolved in dry dioxane (30.0 mL, 0.177 M) and the pressure vessel was degassed with argon for 10 minutes. Tetrakis(triphenylphosphine) palladium (0) (184 mg, 0.159 mmol) was added under argon, vessel sealed, and allowed to stir at 150° C. for 15 hours. The reaction was diluted with ethyl acetate (500 mL) and washed successively with water (3×200 mL) and brine (200 mL). The organic layer was dried with sodium sulfate, filtered, and concentrated under reduced pressure. The crude residue was subjected to flash column chromatography with a 10-80% ethyl acetate and hexanes eluent to yield BH1-103 (1.05 g, 63%) as a yellow solid. ¹H NMR (500 MHz, DMSO-d6) δ 12.57 (s, 1H), 9.40 (t, J=5.5 Hz, 1H), 8.28 (d, J=8.5 Hz, 2H), 8.08 (d, J=8.5 Hz, 2H), 7.76 (d, J=3.6 Hz, 1H), 7.00 (d, J=3.6 Hz, 1H), 4.36 (d, J=5.4 Hz, 2H); HPLC-MS (ESI+) m/z 312.1 (M+H)⁺.

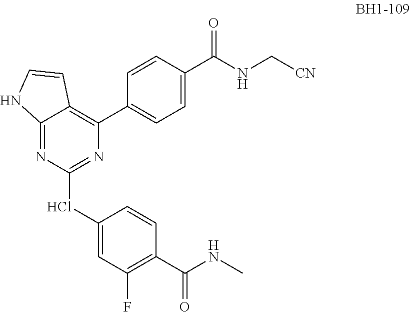

4-((4-(4-((Cyanomethyl)carbamoyl)phenyl)-7H-pyrrolo[2,3-d]pyrimidin-2-yl)amino)-2-fluoro-N-methylbenzamide (BH1-109): This was prepared in the same way as BH1-090 from BH1-103 (50 mg, 0.160 mmol) and 4-amino-3-fluoro-N-methylbenzamide (27.0 mg, 0.160 mmol) with a 16 hour reaction time. The crude material subjected to flash column chromatography with a 40-80% ethyl acetate and hexanes eluent to yield BH1-109 (11 mg, 16%) as a yellow solid. ¹H NMR (500 MHz, DMSO-d6) δ 11.94 (s, 1H), 9.97 (s, 1H), 9.38 (1, J=5.5 Hz, 1H), 8.28 (d, J=8.1 Hz, 2H), 8.16-8.04 (m, 3H), 7.95 (t, J=4.5 Hz, 1H), 7.68-7.56 (m, 2H), 7.42 t, J=2.5 Hz, 1H), 6.79 (dd, J=3.7, 1.6 Hz, 1H), 4.37 (d, J=5.5 Hz, 2H), 2.78 (d, J=4.6 Hz, 3H); ¹⁹F NMR (471 MHz, DMSO-d6) δ −111.90 (dt, J=14.0, 6.3 Hz); HPLC-MS (ESI+) m/z 444.2 (M+H)⁺;)⁺.

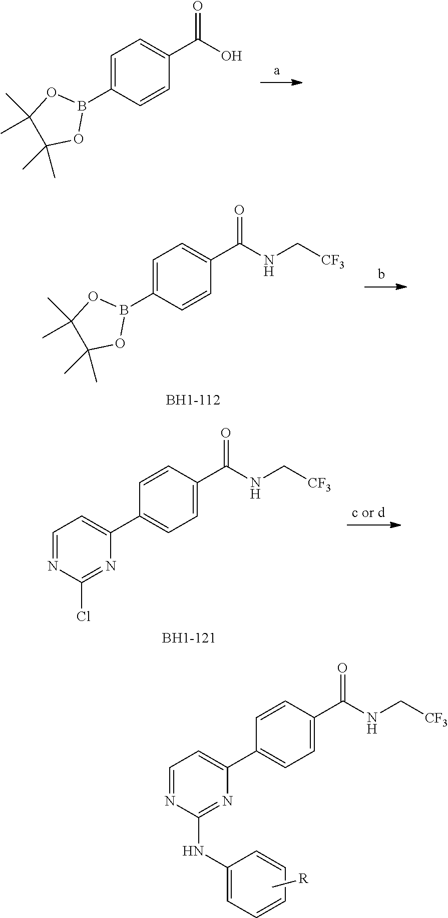

Reagents and Conditions: (a) 2,2,2-trifluoroethan-1-amine hydrochloride, HATU, DIPEA, DMF, r.t., 1.5 h, 94%; (b) 2,4-dichloropyrimidine, Pd (dppf) C₁₂, aq. Cs₂CO₃, 1,4-dioxane, 110° C., 19 h, 50%; (c) substituted aniline, K₂CO₃, XPhos, Pd₂(dba)₃, 1,4-dioxane, 100° C., 16 h; (d) substituted aniline, 4M HCl in 1,4-dioxane, 2-methoxyethanol, 110° C., 16 h.

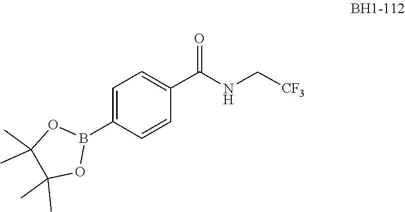 [SMILES: CC1(C)OB(c2ccc(C(=O)NCC(F)(F)F)cc2)OC1(C)C]

4-(4,4,5,5-Tetramethyl-1,3,2-dioxaborolan-2-yl)-N-(2,2,2-trifluoroethyl)benzamide (BH1-112): 4-(4,4,5,5-Tetramethyl-1,3,2-dioxaborolan-2-yl)benzoic acid (1.00 g, 4.03 mmol) and HATU (1.69 g, 4.43 mmol) was dissolved in dry dimethylformamide (30.0 mL, 0.134 M) and round bottom flask degassed with argon 10 minutes with reaction stirring at room temperature. 2,2,2-trifluoroethan-1-amine hydrochloride (601 mg, 4.43 mmol) and dry diisopropylethylamine (1.56 g. 12.1 mmol, 2.10 mL) was added under argon and reaction was allowed to stir under argon atmosphere at room temperature for 1.5 hours. The reaction was diluted with ethyl acetate (500 mL) and washed successively with water (5×200 mL), saturated ammonium chloride (2×100 mL), and brine (3×100 mL). The organic layer was dried with sodium sulfate, filtered, and concentrated under reduced pressure to yield BH1-112 (1.25 g, 94%) as a white solid. ¹H NMR (500 MHz, DMSO-d6) δ 9.16 (t. J=6.3 Hz, 1H), 7.89 (d, J=7.8 Hz, 2H), 7.78 (d, J=7.8 Hz, 2H), 4.09 (qd, J=9.8, 6.3 Hz, 2H), 1.31 (s, 12H); ¹⁹F NMR (471 MHz, DMSO-d6) δ −70.39 (t, J=9.8 Hz); HPLC-MS (ESI+) m/z 330.2 (M+H)⁺.

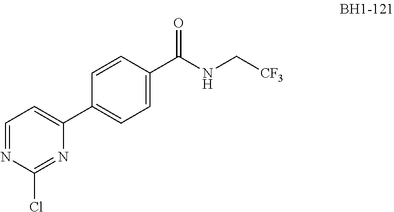 [SMILES: O=C(NCC(F)(F)F)c1ccc(-c2ccnc(Cl)n2)cc1]

4-(2-Chloropyrimidin-4-yl)-N-(2,2,2-trifluoroethyl)benzamide (BH1-121): This was prepared in the same way as BH1-113 from BH1-112 (1.10 g, 3.36 mmol) and 2,4-dichloropyrimdine (500 mg, 3.356 mmol) with a 19 hour reaction time. The crude residue was subjected to flash column chromatography with a 0-40% ethyl acetate and hexanes eluent to yield BH1-121 (234 mg. 50%) as a white solid. ¹H NMR (500 MHz, DMSO-d6) δ 9.29 (t, J=6.3 Hz, 1H), 8.89 (d, J=5.3 Hz, 1H), 8.33 (d, J=8.3 Hz, 2H), 8.25 (d, J=5.3 Hz, 1H), 8.07 (d, J=8.2 Hz, 2H), 4.13 (qd, J=9.7, 6.2 Hz, 2H); ¹⁹F NMR (471 MHz, DMSO-d6) δ −70.33 (t, J=9.6 Hz); HPLC-MS (ESI+) m/z 315.8 (M+H)⁺.

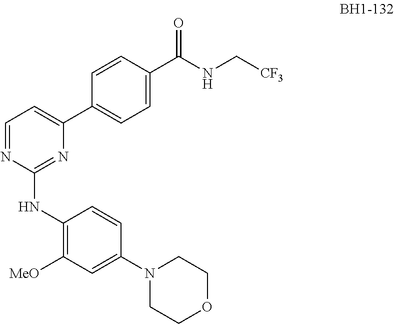 [SMILES: COc1cc(N2CCOCC2)ccc1Nc1nccc(-c2ccc(C(=O)NCC(F)(F)F)cc2)n1]

4-(2-((2-Methoxy-4-morpholinophenyl)amino)pyrimidin-4-yl)-N-(2,2,2-trifluoroethyl)benzamide (BH1-132): This was prepared in the same way as BH1-090 from BH1-121 (50 mg, 0.158 mmol) and 2-methoxy-4-morpholinoaniline (30.0 mg, 0.142 mmol) with a16 hour reaction time. The crude residue was subjected to flash column chromatography with a 40-70% ethyl acetate and hexanes eluent to yield BH1-132 (16.0 mg, 23%) as a tan solid. ¹H NMR (500 MHz, DMSO-d6) δ 9.22 (t, J=6.3 Hz, 1H), 8.49 (d, J=5.2 Hz, 1H), 8.23 (d, J=8.2 Hz, 2H), 8.12 (s, 1H), 8.02 (d, J=8.2 Hz, 2H), 7.83 (d, J=8.7 Hz, 1H), 7.40 (d, J=5.2 Hz, 1H), 6.67 (d, J=2.6 Hz, 1H), 6.54 (dd, J=8.8, 2.5 Hz, 1H), 4.12 (qd, J=9.8, 6.5 Hz, 2H), 3.83 (s, 3H), 3.76 (t, J=4.7 Hz, 4H), 3.11 (t, J=4.7 Hz, 4H); ¹⁹F NMR (471 MHz, DMSO-d6) δ −70.36 (t, J=9.7 Hz); HPLC-MS (ESI+) m/z 488.2 (M+H)⁺; HRMS (TOF) m/z 488.1891 (M+H)⁺.

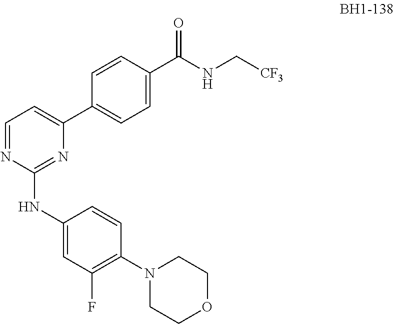 [SMILES: O=C(NCC(F)(F)F)c1ccc(-c2ccnc(Nc3ccc(N4CCOCC4)c(F)c3)n2)cc1]

4-(2-((3-Fluoro-4-morpholinophenyl)amino)pyrimidin-4-yl)-N-(2,2,2-trifluoroethyl)benzamide (BH1-138): This was prepared in the same way as BH1-090 from BH1-121 (75 mg, 0.237 mmol) and 3-fluoro-4-morpholinoaniline (42.0 mg, 0.214 mmol) with a 16 hour reaction time. The crude residue was subjected to flash column chromatography with a 10-30% ethyl acetate and hexanes eluent to yield BH1-138 (42.0 mg, 42%) as a yellow solid. ¹H NMR (500 MHz, DMSO-d6) δ 9.79 (s, 1H), 9.24 (1, J=6.3 Hz, 1H), 8.60 (d, J=5.1 Hz, 1H), 8.27 (d, J=8.4 Hz, 2H), 8.06 (d, J=8.5 Hz, 2H), 7.78 (dd, J=15.4, 2.4 Hz, 1H), 7.59-7.44 (m, 2H), 7.03 (t, J=9.4 Hz, 1H), 4.13 (qd, J=9.8, 6.4 Hz, 2H), 3.74 (t, J=4.7 Hz, 4H), 2.96 (t, J=4.7 Hz, 4H); ¹⁹F NMR (471 MHz, DMSO-d6) δ −70.36 (t, J=9.6 Hz), —122.40 (dd, J=15.4, 9.9 Hz); HPLC-MS (ESI+) m/z 476.1 (M+H)⁺; HRMS (TOF) m/z 476.1695 (M+H)⁺.

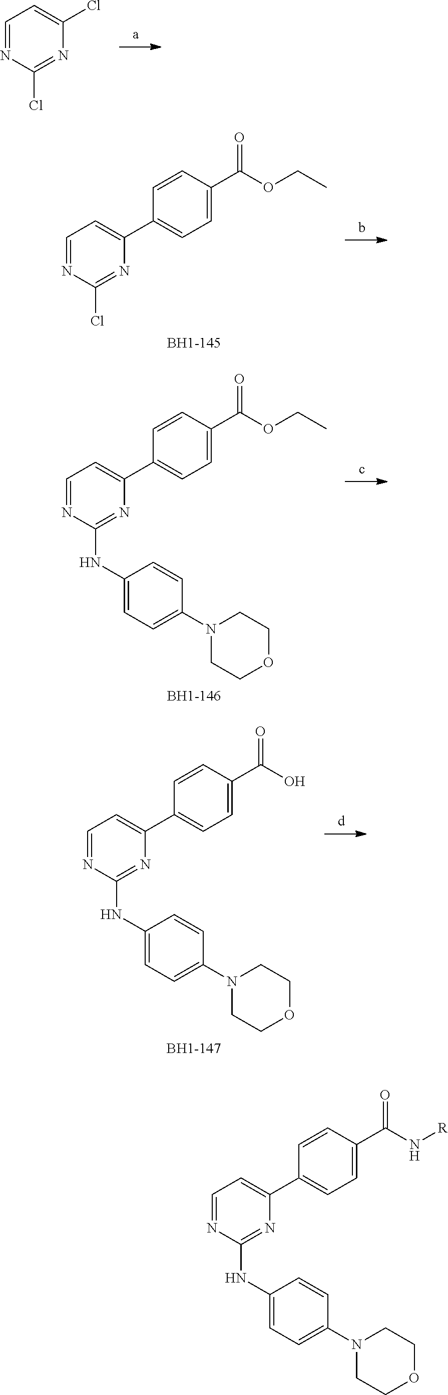

Reagents and Conditions: (a) (4-(ethoxycarbonyl)phenyl)boronic acid, Pd (dppf) Cl₂, aq. Cs₂CO₃, 1,4-dioxane, 100° C., 16 h, 66%; (b) 4-morpholinoaniline, p-toluenesulfonic acid monohydrate, 1,4-dioxane. 110° C., 24 h, 92%; (c) aq. LiOH, 3:1 MeOH: THF, reflux, 1.5 h, 1 M HCl, 83%; (d) RNH₂, HATU, DIPEA, DMF, r.t., 1 h.

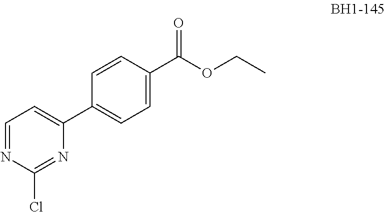 [SMILES: CCOC(=O)c1ccc(-c2ccnc(Cl)n2)cc1]

Ethyl 4-(2-chloropyrimidin-4-yl)benzoate (BH1-145): This was prepared in the same way BH1-113 as from 2,4-dichloropyrimidine (1.00 g, 6.71 mmol) and [4-(ethoxycarbonyl)phenyl]boronic acid (1.30 g, 6.71 mmol) with a 16 h reaction time. The crude residue was subjected to flash column chromatography with a 0-15% ethyl acetate and hexanes eluent to yield BH1-145 (1.16 g, 66%) as a white solid. ¹H NMR (500 MHz, DMSO-d6) δ 8.91 (d, J=5.2 Hz, 1H), 8.33 (d, J=8.5 Hz, 2H), 8.24 (d, J=5.3 Hz, 1H), 8.13 (d, J=8.4 Hz, 2H), 4.36 (q, J=7.1 Hz, 2H), 1.35 (t, J=7.1 Hz, 3H); HPLC-MS (ESI+) m/z 263.1 (M+H)⁺.

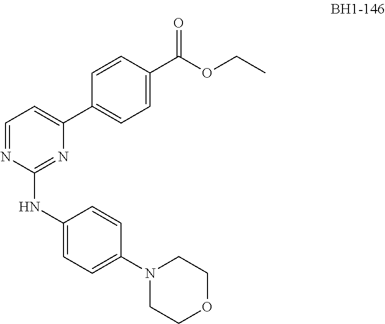 [SMILES: CCOC(=O)c1ccc(-c2ccnc(Nc3ccc(N4CCOCC4)cc3)n2)cc1]

Ethyl 4-(2-((4-morpholinophenyl)amino)pyrimidin-4-yl)benzoate (BH1-146): Ethyl 4-(2-chloropyrimidin-4-yl)benzoate (BH1-145) (800 mg, 3.04 mmol), 4-morpholinoaniline (706 mg, 3.96 mmol), and p-toluenesulfonic acid monohydrate (521 mg, 2.74 mmol) was dissolved in dry dioxane (20.0 mL, 0.152 M) and pressure vessel degassed with argon for 10 minutes. The vessel was sealed and allowed to stir at 110° C. for 30 hours. The reaction was diluted with ethyl acetate (300 mL) then washed successively with water (2×300 mL), saturated sodium bicarbonate (2×100 mL), and brine (2×100 mL). The organic layer was dried with sodium sulfate, filtered and concentrated under reduced pressure to yield BH1-146 (2.84 g, 92%) as a brown solid. ¹H NMR (500 MHz, DMSO-d6) δ 9.52 (s, 1H), 8.55 (d, J=5.1 Hz, 1H), 8.28 (d, J=8.5 Hz, 2H), 8.11 (d, J=8.6 Hz, 2H), 7.67 (d, J=9.0 Hz, 2H), 7.39 (d, J=5.2 Hz, 1H), 6.94 (d, J=9.0 Hz, 2H), 4.36 (q, J=7.1 Hz, 2H), 3.74 (t, J=4.8 Hz, 4H), 3.05 (t, J=4.8 Hz, 4H), 1.35 (t, J=7.1 Hz, 3H); HPLC-MS (ESI+) m/z 405.2 (M+H)⁺.

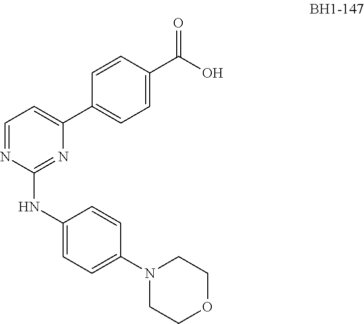 [SMILES: O=C(O)c1ccc(-c2ccnc(Nc3ccc(N4CCOCC4)cc3)n2)cc1]

4-(2-((4-Morpholinophenyl)amino)pyrimidin-4-yl)benzoic acid (BH1-147): Ethyl 4-(2-chloropyrimidin-4-yl)benzoate (3.23 g, 7.97 mmol) was dissolved in a 3:1 ratio of methanol (24.0 mL) and tetrahydrofuran (8.00 mL, 0.250 M). Aqueous lithium hydroxide (401 mg, 8.40 mL, 2.00 M) was added and the round bottom flask was allowed to heat at reflux for 1.5 hours. The reaction was cooled to room temperature and concentrated under reduced pressure. The resulting slurry was neutralized with aqueous hydrochloric acid (16.7 mL, 1.00 M) and diluted with water (20 mL). The solid was filtered off via vacuum filtration to yield a muddy solid. The resulting residue was triturated with methanol (40 mL) and then diethyl ether (40 mL) to yield BH1-147 (2.50 g, 83%) as a brown powder. ¹H NMR (500 MHz, DMSO-d6) δ 13.19 (s, 1H), 9.50 (s, 1H), 8.54 (d, J=5.2 Hz, 1H), 8.25 (d, J=8.2 Hz, 2H), 8.08 (d, J=8.3 Hz, 2H), 7.67 (d, J=9.0 Hz, 2H), 7.38 (d, J=5.2 Hz, 1H), 6.94 (d, J=9.1 Hz, 2H), 3.74 (t, J=5.4 Hz, 4H), 3.05 (t, J=5.4 Hz, 4H); HPLC-MS (ESI+) m/z 377.2 (M+H)⁺.

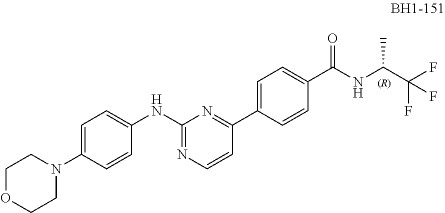

(R)-4-(2-((4-Morpholinophenyl)amino)pyrimidin-4-yl)-N-(1,1,1-trifluoropropan-2-yl)benzamide (BH1-151): 4-(2-((4-Morpholinophenyl)amino)pyrimidin-4-yl)benzoic acid (BH1-147) (100 mg, 0.265 mmol) and HATU (111 mg, 0.292 mmol) was dissolved in dry dimethylformamide (0.500 mL, 0.530 M) and round bottom flask was degassed with argon for 10 minutes. (R)-1,1,1-Trifluoropropan-2-amine hydrochloride (44.0 mg, 0.292 mmol) and diisopropylethylamine (102 mg, 0.795 mmol, 0.140 mL) were added under argon and reaction was allowed to stir at room temperature for 1 hour under an argon atmosphere. The reaction mixture was diluted with ethyl acetate (50 mL) and washed successively with water (2×50 mL), saturated ammonium chloride (2×50 mL), and brine (2×50 mL). The organic layer was dried with sodium sulfate, filtered, and concentrated under reduced pressure to yield BH1-151 (88.0 mg, 70%) as a yellow solid. ¹H NMR (500 MHz, DMSO-d6) δ 9.50 (s, 1H), 8.98 (d, J=8.7 Hz, 1H), 8.54 (d, J=5.1 Hz, 1H), 8.26 (d, J=8.5 Hz, 2H), 8.04 (d, J=8.4 Hz, 2H), 7.67 (d, J=9.0 Hz, 2H), 7.40 (d, J=5.2 Hz, 1H), 6.93 (d, J=9.1 Hz, 2H), 4.89 (h, J=7.7 Hz, 1H), 3.74 (t, J=4.8 Hz, 4H), 3.05 (t, J=4.9 Hz, 4H), 1.39 (d, J=7.1 Hz, 3H); ¹⁹F NMR (471 MHz, DMSO-d6) δ −75.56 (d, J=7.9 Hz); HPLC-MS (ESI+) m/z 472.2 (M+H)⁺; HRMS (TOF) m/z 472.1946 (M+H)⁺.

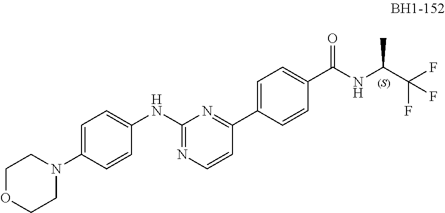

(S)-4-(2-((4-Morpholinophenyl)amino)pyrimidin-4-yl)-N-(1,1,1-trifluoropropan-2-yl)benzamide (BH1-152): This was prepared in the same way as BH1-151 from BH1-148 (100 mg, 0.266 mmol) and(S)-1,1,1-trifluoropropan-2-amine hydrochloride (44.0 mg, 0.293 mmol) to yield BH1-152 (120 mg, 96%) as a yellow solid. ¹H NMR (500 MHz, DMSO-d6) δ 9.51 (s, 1H), 8.99 (d, J=8.7 Hz, 1H), 8.55 (d, J=5.2 Hz, 1H), 8.27 (d, J=8.4 Hz, 2H), 8.05 (d, J=8.5 Hz, 2H), 7.68 (d, J=9.0 Hz, 2H), 7.41 (d, J=5.2 Hz, 1H), 6.94 (d, J=9.1 Hz, 2H), 4.90 (h, J=7.7 Hz, 1H), 3.75 (t, J=4.9 Hz, 4H), 3.06 (t, J=4.9 Hz, 4H), 1.40 (d, J=7.1 Hz, 3H); ¹⁹F NMR (471 MHz, DMSO-d6) δ −75.56 (d, J=7.9 Hz); HPLC-MS (ESI+) m/z 472.2 (M+H)⁺;)⁺; HRMS (TOF) m/z 472.1943 (M+H)⁺.

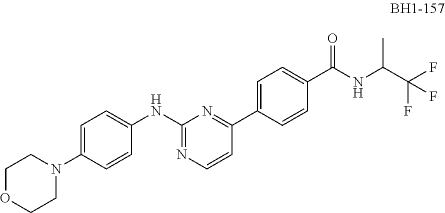 [SMILES: CC(NC(=O)c1ccc(-c2ccnc(Nc3ccc(N4CCOCC4)cc3)n2)cc1)C(F)(F)F]

(RS)-4-(2-((4-Morpholinophenyl)amino)pyrimidin-4-yl)-N-(1,1,1-trifluoropropan-2-yl)benzamide (BH1-157/SR8-170B2): This was prepared in the same way as BH1-151 from BH1-148 (100 mg, 0.266 mmol) and 1,1,1-trifluoropropan-2-amine hydrochloride (44 mg, 0.292 mmol) to yield SR8-170B2 (81.0 mg, 65%) as a yellow solid. ¹H NMR (500 MHz, DMSO-d6) δ 9.50 (s, 1H), 8.98 (d, J=8.7 Hz, 1H), 8.54 (d, J=5.1 Hz, 1H), 8.26 (d, J=8.6 Hz, 2H), 8.04 (d, J=8.6 Hz, 2H), 7.67 (d, J=9.0 Hz, 2H), 7.40 (d, J=5.1 Hz, 1H), 6.93 (d, J=9.1 Hz, 2H), 4.89 (h, J=7.6 Hz, 1H), 3.75 (t, J=5.0 Hz, 4H), 3.05 (t, J=5.0 Hz, 4H), 1.39 (d, J=7.1 Hz, 3H); ¹⁹F NMR (471 MHz, DMSO-d6) δ −75.56 (d, J=7.9 Hz); HPLC-MS (ESI+) m/z 472.3 (M+H)⁺;)⁺; HRMS (TOF) m/z 472.1945 (M+H)⁺.

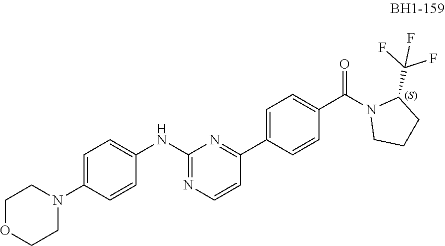

(S)-(4-(2-((4-Morpholinophenyl)amino)pyrimidin-4-yl)phenyl) (2-(trifluoromethyl)pyrrolidin-1-yl) methanone (BH1-159): This was prepared in the same way as BH1-151 from BH1-148 (100 mg, 0.266 mmol) and(S)-2-(trifluoromethyl)pyrrolidine (42.0 mg, 0.292 mmol) to yield BH1-159 (115 mg, 87%) as a yellow solid. ¹H NMR (500 MHz, DMSO-d6) δ 9.49 (s, 1H), 8.53 (d, J=5.1 Hz, 1H), 8.23 (d, J=8.4 Hz, 2H), 7.71 (d, J=8.0 Hz, 2H), 7.67 (d, J=9.1 Hz, 2H), 7.38 (d, J=5.2 Hz, 1H), 6.93 (d, J=9.1 Hz, 2H), 5.08 (s, 1H), 3.74 (t, J=4.8 Hz, 4H), 3.63 (d, J=9.2 Hz, 1H), 3.45 (s, 1H), 3.05 (t, J=4.8 Hz, 4H), 2.23 (s, 1H), 2.08-1.93 (m, 2H), 1.88 (s, 1H); ¹⁹F NMR (471 MHz, DMSO-d6) δ −73.22 (d, J=8.1 Hz); HPLC-MS (ESI+) m/z 498.3 (M+H)⁺; HRMS (TOF) m/z 498.2100 (M+H)⁺.

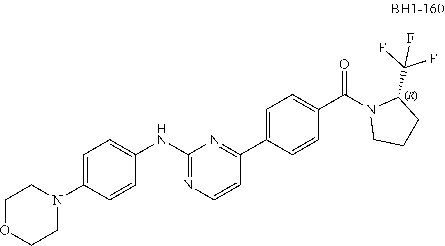

(R)-(4-(2-((4-Morpholinophenyl)amino)pyrimidin-4-yl)phenyl) (2-(trifluoromethyl)pyrrolidin-1-yl) methanone (BH1-160): This was prepared in the same way as BH1-151 from BH1-148 (100 mg, 0.266 mmol) and (R)-2-(trifluoromethyl)pyrrolidine (42.0 mg, 0.292 mmol) with a 2 hour reaction time to yield BH1-160 (85 mg, 64%) as a yellow solid. ¹H NMR (500 MHz, DMSO-d6) δ 9.49 (s, 1H), 8.53 (d, J=5.1 Hz, 1H), 8.23 (d, J=8.4 Hz, 2H), 7.71 (d, J=8.0 Hz, 2H), 7.67 (d, J=9.0 Hz, 2H), 7.38 (d, J=5.2 Hz, 1H), 6.93 (d, J=9.1 Hz, 2H), 5.08 (s, 1H), 3.74 (t, J=5.1 Hz, 4H), 3.63 (d, J=9.3 Hz, 1H), 3.45 (s, 1H), 3.05 (t, J=5.0 Hz, 4H), 2.23 (s, 1H), 2.08-1.93 (m, 2H), 1.89 (d, J=15.1 Hz, 1H); ¹⁹F NMR (471 MHz, DMSO-d6) δ −73.22 (d, J=8.1 Hz); HPLC-MS (ESI+) m/z 498.3 (M+H)⁺; HRMS (TOF) m/z 498.2101 (M+H)⁺.

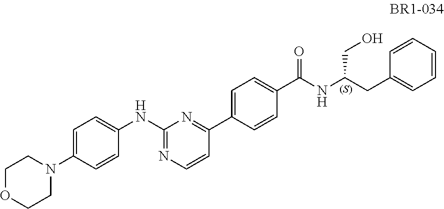

(S)—N-(1-Hydroxy-3-phenylpropan-2-yl)-4-(2-((4-morpholinophenyl)amino)pyrimidin-4-yl)benzamide (BR1-034): This was prepared in the same way as BH1-151 from BH1-148 (100 mg, 0.266 mmol) and L-phenylalaninol (44.0 mg, 0.292 mmol) with a 2 hour reaction time. The crude residue was subjected to flash column chromatography with a 0-5% methanol:dichloromethane eluent to yield BR1-034 (86.0 mg, 63%) as a yellow solid. ¹H NMR (500 MHz, DMSO-d6) δ 9.47 (s, 1H), 8.52 (d, J=5.2 Hz, 1H), 8.30 (d, J=8.4 Hz, 1H), 8.20 (d, J=8.6 Hz, 2H), 7.94 (d, J=8.5 Hz, 2H), 7.67 (d, J=9.0 Hz, 2H), 7.38 (d, J=5.2 Hz, 1H), 7.30-7.24 (m, 4H), 7.16 (tt, J=5.7, 2.5 Hz, 1H), 6.93 (d, J=9.1 Hz, 2H), 4.86 (t, J=5.7 Hz, 1H), 4.19 (tq, J=8.7, 5.6 Hz, 1H), 3.74 (t, J=4.9 Hz, 4H), 3.52 (dt, J=10.9, 5.5 Hz, 1H), 3.45 (dt, J=10.8, 6.0 Hz, 1H), 3.05 (t, J=4.8 Hz, 4H), 2.97 (dd, J=13.7, 5.2 Hz, 1H), 2.81 (dd, J=13.7, 9.0 Hz, 1H); HPLC-MS (ESI+) m/z 511.3 (M+H)⁺; HRMS (TOF) m/z 510.2493 (M+H)⁺.

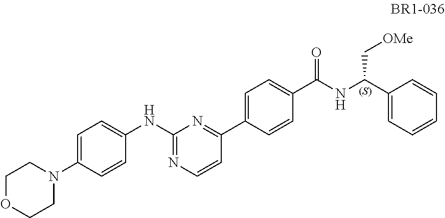

(S)—N-(2-Methoxy-1-phenylethyl)-4-(2-((4-morpholinophenyl)amino)pyrimidin-4-yl)benzamide (BR1-036): This was prepared in the same way as BH1-151 from BH1-148 (100 mg, 0.266 mmol) and(S)-2-methoxy-1-phenylethanamine (44.0 mg, 0.292 mmol) with a 2 hour reaction time. The crude residue was subjected to flash column chromatography with a 0-5% methanol:dichloromethane eluent to yield BR1-036 (39.0 mg, 29%) as a yellow solid. ¹H NMR (500 MHz, DMSO-d6) δ 9.49 (s, 1H), 8.97 (d, J=8.2 Hz, 1H), 8.53 (d, J=5.1 Hz, 1H), 8.24 (d, J=8.5 Hz, 2H), 8.04 (d, J=8.4 Hz, 2H), 7.68 (d, J=9.0 Hz, 2H), 7.45 (d, 7.4 Hz, 2H), 7.39 (d, J=5.2 Hz, 1H), 7.35 (t, J=7.6 Hz, 2H), 7.27 (tt, J=7.4, 1.4 Hz, 1H), 6.93 (d, J=9.1 Hz, 2H), 5.30 (td, J=8.5, 5.2 Hz, 1H), 3.81-3.69 (m, 5H), 3.57 (dd, J=4.8, 5.3 Hz, 1H), 3.05 (t, J=4.8 Hz, 4H); HPLC-MS (ESI+) m/z 510.9 (M+H)⁺; HRMS (TOF) m/z 510.2493 (M+H)⁺.

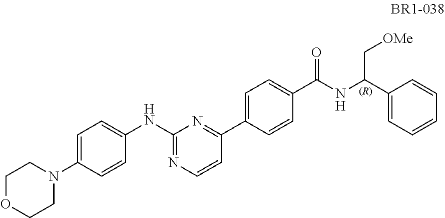

(R)—N-(2-Methoxy-1-phenylethyl)-4-(2-((4-morpholinophenyl)amino)pyrimidin-4-yl)benzamide (BR1-038): This was prepared in the same way as BH1-151 from BH1-148 (100 mg, 0.266 mmol) and (R)-2-methoxy-1-phenylethanamine (44.0 mg, 0.292 mmol) with a 2 hour reaction time. The crude residue was subjected to flash column chromatography with a 0-5% methanol:dichloromethane eluent to yield BR1-038 (27.0 mg, 20%) as a yellow solid. ¹H NMR (500 MHz, DMSO-d6) δ 9.49 (s, 1H), 8.97 (d, J=8.2 Hz, 1H), 8.53 (d, J=5.1 Hz, 1H), 8.24 (d, J=8.5 Hz, 2H), 8.04 (d, J=8.5 Hz, 2H), 7.68 (d, J=9.0 Hz, 2H), 7.44 (d, J=7.3 Hz, 2H), 7.39 (d, J=5.2 Hz, 1H), 7.35 (t, J=7.6 Hz, 2H), 7.27 (tt, J=7.3, 1.4 Hz, 1H), 6.93 (d, J=9.1 Hz, 1H), 5.30 (td, J=8.5, 5.3 Hz, 1H), 3.82-3.68 (m, 5H), 3.57 (dd, J=5.3, 4.8 Hz, 1H), 3.05 (t, J=4.9 Hz, 6H); HPLC-MS (ESI+) m/z 510.3 (M+H)⁺; HRMS (TOF) m/z 510.2492 (M+H)⁺.

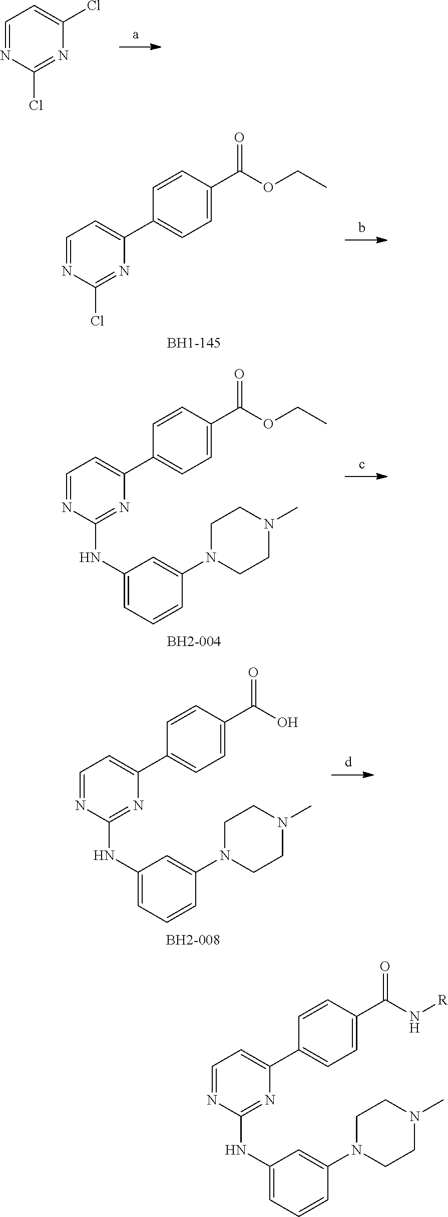

Reagents and Conditions: (a) (4-(ethoxycarbonyl)phenyl)boronic acid, Pd (dppf) Cl₂, aq. Cs₂CO₃, 1,4-dioxane, 100° C., 16 h, 66%; (b) 3-(4-methylpiperazin-1-yl) aniline, 4 M HCl in 1,4-dioxane, 110° C., 30 h, 69%; (c) aq. LiOH, 3:1 MeOH: THE, reflux, 2 h, 1 M HCl, 66%; (d) amine (RNH₂), HATU, DIPEA, DMF, r.t., 2 h.

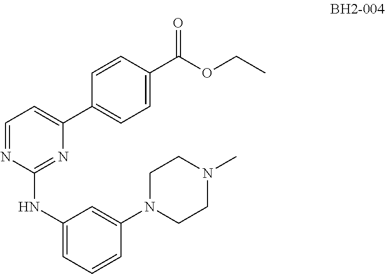 [SMILES: CCOC(=O)c1ccc(-c2ccnc(Nc3cccc(N4CCN(C)CC4)c3)n2)cc1]

Ethyl 4-(2-((3-(4-methylpiperazin-1-yl)phenyl) amino)pyrimidin-4-yl)benzoate (BH2-004): Ethyl 4-(2-chloropyrimidin-4-yl)benzoate (BH1-145) (50.0 mg, 0.190 mmol) and 3-(4-methylpiperazin-1-yl) aniline (40.0 mg, 0.209 mmol) were dissolved in 2-methoxyethanol (2.00 mL, 0.095 M). Hydrochloric acid in dioxane (4M, 0.05 mL) was added to a microwave vial, vial was sealed, and allowed to stir at 110° C. for 30 hours. The solvent was evaporated under reduced pressure and subsequently diluted with ethyl acetate (50 mL). The organic layer was washed with saturated sodium bicarbonate (50 mL) and aqueous layer was further extracted with ethyl acetate (2×50 mL). The organic layer was dried with sodium sulfate, filtered, and concentrated under reduced pressure. The crude residue was subjected to flash column chromatography with a 10-50% ethyl acetate: hexanes eluent to yield BH2-004 (54.0 mg, 69%) as a tan solid. ¹H NMR (500 MHz, DMSO-d6) δ 9.59 (s, 1H), 8.60 (d, J=5.1 Hz, 1H), 8.30 (d, J=8.2 Hz, 2H), 8.10 (d, J=8.4 Hz, 2H), 7.60 (s, 1H), 7.46 (d, J=5.1 Hz, 1H), 7.23 (dd, J=8.0, 1.9 Hz, 1H), 7.14 (t, J=8.1 Hz, 1H), 6.58 (dd, J=8.1, 2.3 Hz, 1H), 4.36 (q, J=7.1 Hz, 2H), 3.15 (t, J=5.0 Hz, 4H), 2.47 (t, J=5.0 Hz, 4H), 2.23 (s, 3H), 1.35 (t, J=7.1 Hz, 3H); HPLC-MS (ESI+) m/z 417.8 (M+H)⁺.

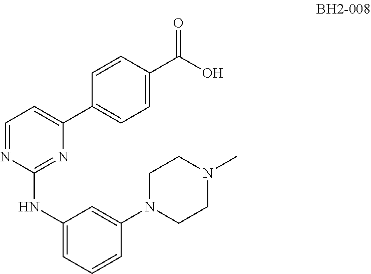 [SMILES: CN1CCN(c2cccc(Nc3nccc(-c4ccc(C(=O)O)cc4)n3)c2)CC1]

4-(2-((3-(4-Methylpiperazin-1-yl)phenyl) amino)pyrimidin-4-yl)benzoic acid (BH2-008): Ethyl 4-(2-((3-(4-methylpiperazin-1-yl)phenyl) amino)pyrimidin-4-yl)benzoate (BH1-145) (433 mg, 1.04 mmol) was dissolved in a suspension of 3:1 ratio of methanol (3.00 mL) and tetrahydrofuran (1.00 mL). An aqueous solution of 2 M lithium hydroxide (1.10 mL) was added and the reaction was heated at reflux for 2 hours. The reaction mixture was neutralized with 1 M HCl in water (2.18 mL, 2.18 mmol) and the solid was filtered over a fritted funnel. The residue was washed subsequently with water (50 mL), methanol (50 mL), and diethyl ether (20 mL) to yield BH2-008 (265 mg, 66%) as a brown solid. ¹H NMR (500 MHz, DMSO-d6) δ 9.59 (s, 1H), 8.60 (d, J=5.2 Hz, 1H), 8.28 (d, J=8.0 Hz, 2H), 8.08 (d, J=8.0 Hz, 2H), 7.61 (s, 1H), 7.46 (d, J=5.2 Hz, 1H), 7.23 (d, J=7.9 Hz, 1H), 7.14 (t, J=8.1 Hz, 1H), 6.59 (d, J=8.2 Hz, 1H), 3.17 (s, 4H), 2.28 (s, 3H); HPLC-MS (ESI+) m/z 390.8 (M+H)⁺.

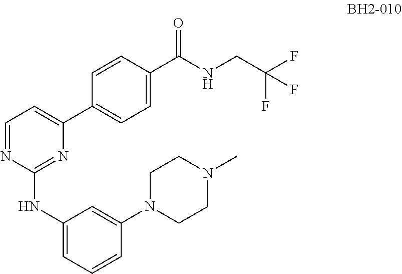 [SMILES: CN1CCN(c2cccc(Nc3nccc(-c4ccc(C(=O)NCC(F)(F)F)cc4)n3)c2)CC1]

4-(2-((3-(4-Methylpiperazin-1-yl)phenyl) amino)pyrimidin-4-yl)-N-(2,2,2-trifluoroethyl)benzamide (BH2-010): 4-(2-((3-(4-methylpiperazin-1-yl)phenyl) amino)pyrimidin-4-yl)benzoic acid (BH2-008) (70.0 mg, 0.180 mmol), HATU (75.0 mg, 0.198 mmol), and 2,2,2-trifluoroethan-1-amine hydrochloride (27.0 mg, 0.198 mmol) were dissolved in dry dimethylformamide (0.500 mL, 0.360 M) and the round bottom flask was degassed with argon for 10 minutes. Dry N,N-diisopropylethylamine (70.0 mg, 0.540 mmol, 95.0 μL) was added under argon and the reaction was allowed to stir at room temperature in under inert conditions for 16 hours. The reaction mixture was diluted with ethyl acetate (50 mL) and washed successively with water (2×30 mL), saturated ammonium chloride (30 mL) and brine (20 mL). The organic layer was dried with sodium sulfate, filtered, and concentrated under reduced pressure. The crude residue was subjected to flash column chromatography with a 0-5% methanol:dichloromethane eluent to yield BH2-010 (30.0 mg, 36%) as a yellow solid. ¹H NMR (500 MHz, DMSO-d6) δ 9.57 (s, 1H), 9.24 (t, J=6.3 Hz, 1H), 8.59 (d, J=5.1 Hz, 1H), 8.29 (d, J=8.4 Hz, 2H), 8.05 (d, J=8.4 Hz, 2H), 7.60 (t, J=2.2 Hz, 1H), 7.47 (d, J=5.2 Hz, 1H), 7.24 (d, J=8.3 Hz, 1H), 7.14 (t, J=8.1 Hz, 1H), 6.58 (dd, J=8.2, 2.3 Hz, 1H), 4.13 (qd, J=9.7, 6.2 Hz, 2H), 3.17 (s, 4H), 2.27 (s, 3H); ¹⁹F NMR (471 MHz, DMSO-d6) δ −70.36 (t, J=9.6 Hz); HPLC-MS (ESI+) m/z 471.9 (M+H)⁺; HRMS (TOF) m/z 471.2110 (M+H)⁺.

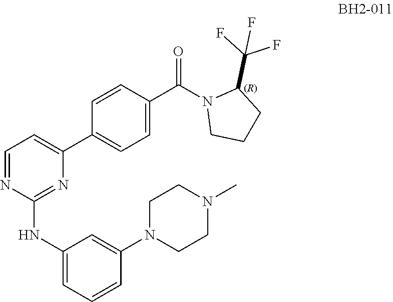

(R)-(4-(2-((3-(4-Methylpiperazin-1-yl)phenyl) amino)pyrimidin-4-yl)phenyl) (2-(trifluoromethyl)pyrrolidin-1-yl) methanone (BH2-011): This was prepared in the same way as BH2-010 from BH2-008 (70.0 mg, 0.180 mmol) and (R)-2-(trifluoromethyl)pyrrolidine (28.0 mg, 0.198 mmol) with a 2 hour reaction time. The crude residue was subjected to flash column chromatography with a 0-4% methanol:dichloromethane eluent to yield BH2-011 (50.0 mg, 54%) as a yellow solid. ¹H NMR (500 MHz, DMSO-d6) δ 9.57 (s, 1H), 8.58 (d, J=5.1 Hz, 1H), 8.25 (d, J=8.1 Hz, 2H), 7.70 (d, J=8.0 Hz, 2H), 7.63 (s, 1H), 7.44 (d, J=5.2 Hz, 1H), 7.23 (dd, J=8.0, 1.9 Hz, 1H), 7.13 (t, J=8.1 Hz, 1H), 6.58 (dd, J=8.2, 2.3 Hz, 1H), 5.08 (s, 1H), 3.62 (d, J=9.1 Hz, 1H), 3.46 (s, 1H), 3.15 (t, J=4.9 Hz, 4H), 2.24 (s, 4H), 2.06-1.93 (m, 3H), 1.89 (s, 1H); 19F NMR (471 MHz, DMSO-d6) δ −73.22 (d, J=7.9 Hz); HPLC-MS (ESI+) m/z 511.9 (M+H)⁺; HRMS (TOF) m/z 511.2420 (M+H)⁺.

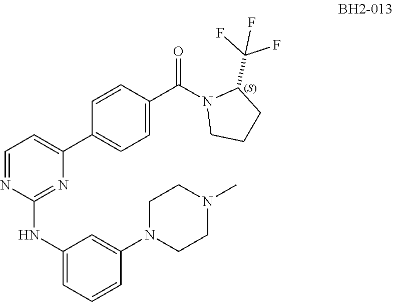

(S)-(4-(2-((3-(4-Methylpiperazin-1-yl)phenyl) amino)pyrimidin-4-yl)phenyl) (2-(trifluoromethyl)pyrrolidin-1-yl) methanone (BH2-013): This was prepared in the same way as BH2-010 from BH2-008 mg, 0.180 mmol) and(S)-2-(trifluoromethyl)pyrrolidine (28.0 mg, 0.198 mmol) with a 1 hour reaction time. The crude residue was subjected to flash column chromatography with a 0-5% methanol:dichloromethane eluent to yield BH2-013 (52.0 mg, 57%) as a yellow solid. ¹H NMR (500 MHz, DMSO-d6) δ 9.57 (s, 1H), 8.59 (d, J=5.1 Hz, 1H), 8.25 (d, J=8.2 Hz, 2H), 7.70 (d, J=7.9 Hz, 2H), 7.63 (s, 1H), 7.44 (d, J=5.2 Hz, 1H), 7.23 (d, J=8.1 Hz, 1H), 7.13 (t, J=8.1 Hz, 1H), 6.58 (dd, J=8.2, 2.3 Hz, 1H), 5.08 (s, 1H), 3.62 (d, J=8.9 Hz, 1H), 3.46 (s, 1H), 3.15 (t, J=5.0 Hz, 4H), 2.24 (s, 4H), 2.00 (td, J=17.8, 17.3, 10.9 Hz, 3H), 1.89 (s, 1H); ¹⁹F NMR (471 MHz, DMSO-d6) δ −73.22 (d, J=7.9 Hz); HPLC-MS (ESI+) m/z 511.9 (M+H)⁺; HRMS (TOF) m/z 511.2421 (M+H)⁺.

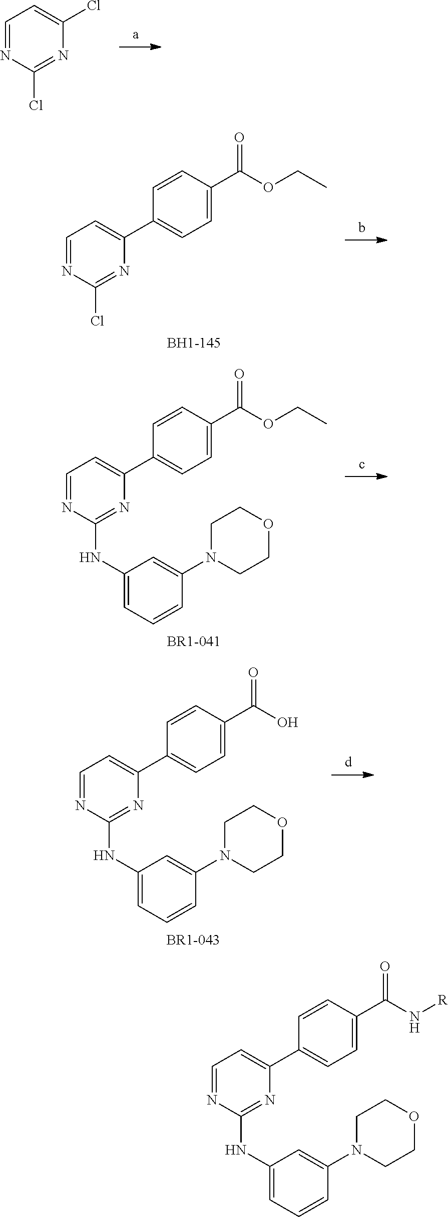

Reagents and Conditions: (a) (4-(ethoxycarbonyl)phenyl)boronic acid, Pd (dppf) Cl₂, aq. Cs₂CO₃, 1,4-dioxane, 100° C., 16 h, 66%; (b) 3-morpholinoaniline, p-toluenesulfonic acid monohydrate, 1,4-dioxane, 110° C., 21 h, 49%; (c) aq. LiOH, 3:1 MeOH: THF, reflux, 1.5 h, 1 M HCl, 70%; (d) amine, HATU, DIPEA, DMF, r.t., 2 h.

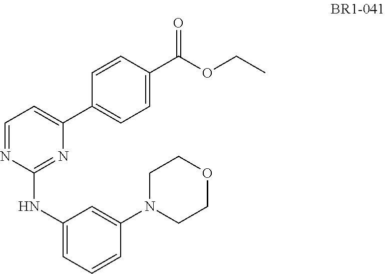 [SMILES: CCOC(=O)c1ccc(-c2ccnc(Nc3cccc(N4CCOCC4)c3)n2)cc1]

Ethyl 4-(2-((3-morpholinophenyl)amino)pyrimidin-4-yl)benzoate (BR1-041): This was prepared in the same way as BH1-146 from BH1-145 (100 mg, 0.381 mmol) and 3-morpholinoaniline (88.0 mg, 0.495 mmol) with a 21 hour reaction time. The crude residue was subjected to flash column chromatography with a 10-50% ethyl acetate: hexanes eluent to yield BR1-041 (75.0 mg, 49%) as a yellow solid. ¹H NMR (500 MHz, DMSO-d6) δ 9.62 (s, 1H), 8.61 (d, J=5.2 Hz, 1H), 8.30 (d, J=8.5 Hz, 2H), 8.10 (d, J=8.5 Hz, 2H), 7.59 (t, J=2.2 Hz, 1H), 7.46 (d, J=5.1 Hz, 1H), 7.27 (dd, J=6.9, 1.2 Hz, 1H), 7.16 (t, J=8.1 Hz, 1H), 6.59 (dd, J=6.3, 2.0 Hz, 1H), 4.36 (q, J=7.1 Hz, 2H), 3.76 (t, J=4.9 Hz, 4H), 3.12 (t, J=4.8 Hz, 4H), 1.35 (t, J=7.1 Hz, 3H); HPLC-MS (ESI+) m/z 404.8 (M+H)⁺.

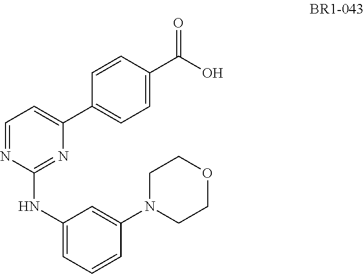 [SMILES: O=C(O)c1ccc(-c2ccnc(Nc3cccc(N4CCOCC4)c3)n2)cc1]

4-(2-((3-Morpholinophenyl)amino)pyrimidin-4-yl)benzoic acid (BR1-043): This was prepared in the same way as BH2-008 from BR1-041 (318 mg, 0.787 mmol) to yield BR1-043 (207 mg, 70%) as a yellow solid. ¹H NMR (500 MHz, DMSO-d6) δ 13.19 (s, 1H), 9.61 (s, 1H), 8.60 (d, J=5.1 Hz, 1H), 8.28 (d, J=8.1 Hz, 2H), 8.08 (d, J=8.1 Hz, 2H), 7.62 (s, 1H), 7.46 (d, J=5.2 Hz, 1H), 7.26 (d, J=7.9 Hz, 1H), 7.16 (t, J=8.1 Hz, 1H), 6.59 (d, J=8.1 Hz, 1H), 3.76 (t, J=4.7 Hz, 4H), 3.12 (t, J=4.7 Hz, 4H); HPLC-MS (ESI+) m/z 376.8 (M+H)⁺.

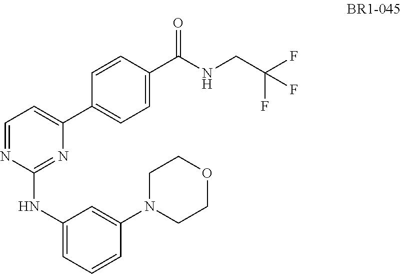 [SMILES: O=C(NCC(F)(F)F)c1ccc(-c2ccnc(Nc3cccc(N4CCOCC4)c3)n2)cc1]

4-(2-((3-Morpholinophenyl)amino)pyrimidin-4-yl)-N-(2,2,2-trifluoroethyl)benzamide (BR1-045): This was prepared in the same way as BH2-010 from BR1-043 (50.0 mg, 0.133 mmol) and 2,2,2-trifluoroethan-1-amine hydrochloride (20.0 mg, 0.146 mmol) with a 2 hour reaction time. The crude residue was subjected to flash column chromatography with a 0-5% methanol:dichloromethane eluent to yield BR1-045 (20.0 mg, 33%) as a yellow solid. ¹H NMR (500 MHz, DMSO-d6) δ 9.60 (s, 1H), 9.24 (t, J=6.3 Hz, 1H), 8.60 (d, J=5.1 Hz, 1H), 8.29 (d, J=8.1 Hz, 2H), 8.05 (d, J=8.1 Hz, 2H), 7.63 (s, 1H), 7.48 (d, J=5.1 Hz, 1H), 7.26 (d, J=8.0 Hz, 1H), 7.16 (t, J=8.1 Hz, 1H), 6.59 (dd, J=8.1, 2.3 Hz, 1H), 4.13 (qd, J=9.7, 6.1 Hz, 2H), 3.77 (t, J=4.7 Hz, 4H), 3.12 (t, J=4.8 Hz, 4H); ¹⁹F NMR (471 MHz, DMSO-d6) δ −70.34 (t, J=9.8 Hz); HPLC-MS (ESI+) m/z 457.8 (M+H)⁺; HRMS (TOF) m/z 458.1795 (M+H)⁺.

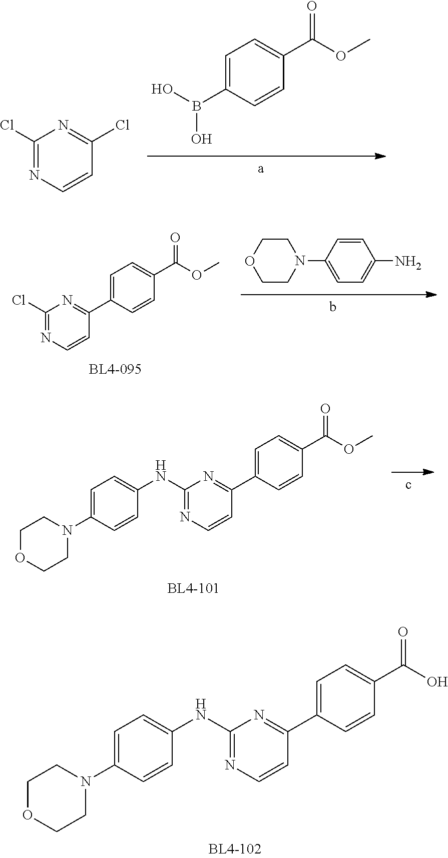

Reagents and Conditions: (a) Pd(PPh₃)₂Cl₂, Na₂CO₃, DMF, dioxane, H₂O, argon, 80° C., overnight, 66%; (b) TsOH, dioxane, reflux, 20 h; (c) LiOH, THF, H₂O, reflux, 4 h, 64% (overall, two steps).

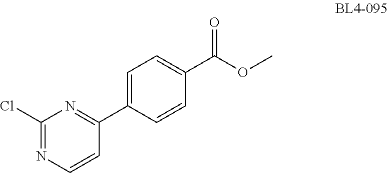 [SMILES: COC(=O)c1ccc(-c2ccnc(Cl)n2)cc1]

Methyl 4-(2-chloropyrimidin-4-yl)benzoate (BL4-095) 2: To a 20 mL microwave vial was added 2,4-dichloropyrimidine (200 mg, 1.34 mmol, 1.0 eq.), (4-(methoxycarbonyl)phenyl)bor-onic acid (290 mg, 1.61 mmol, 1.2 eq.), Na₂CO₃ (426 mg. 4.02 mmol, 3.0 eq.), water (0.2 mL), 1,4-dioxane (0.5 mL), and DMF (0.8 mL). The mixture was degassed by bubbling argon for 5 minutes before Pd(PPh₃)₄ (94 mg, 0.13 mmol, 10% mmol) was added. The mixture was heated to 80° C. under argon for 10 h. The reaction mixture was gradually cooled down to ambient temperature before being quenched with water (10 mL). The residue was diluted with ethyl acetate (20 mL) and two layers were separated, and the aqueous layer was extracted with ethyl acetate (3×15 mL). The combined organic layers were washed with brine (10 mL), dried (Na₂SO₄) and filtered. The residue was concentrated under reduced pressure, and the crude was purified by column chromatography (ethyl acetate 0 to 40% in hexanes) to give BL4-095 (220 mg, 66%) as a yellow solid. ¹H NMR (500 MHz, DMSO) δ 8.91 (d, J=5.5 Hz, 1H), 8.34 (d, J=9.0 Hz, 2H), 8.25 (d, J=5.5 Hz, 1H), 8.14 (d, J=9.0 Hz, 2H), 3.91 (s, 3H). HPLC-MS (ESI+): m/z 248.9 (M+H)⁺.

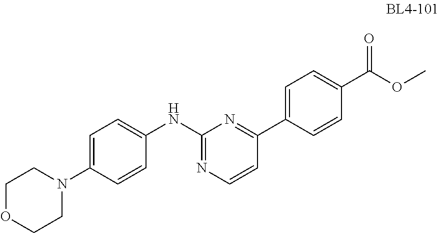 [SMILES: COC(=O)c1ccc(-c2ccnc(Nc3ccc(N4CCOCC4)cc3)n2)cc1]

Methyl 4-(2-((4-morpholinophenyl)amino)pyrimidin-4-yl)benzoate (BL4-101) 2: To a 10 mL microwave vial was added BL4-095 (100 mg, 0.4 mmol, 1.0 eq.), 4-morpholinoaniline (79 mg, 0.44 mmol, 1.1 eq.), and dioxane (2 mL), followed by adding TsOH·H₂O (84.4 mg, 0.44 mmol, 1.1 eq.). The mixture was heated to 110° C. for 1 d. The reaction mixture was gradually cooled to ambient temperature before being quenched with H₂O (10 mL). The residue was diluted with ethyl acetate (20 mL) and two layers were separated, and the aqueous layer was extracted with ethyl acetate (3×15 mL). The combined organic layers were washed with brine (10 mL), dried (Na₂SO₄) and filtered. The residue was concentrated under reduced pressure to give BL4-101, which was used for next step without further purification. ¹H NMR (500 MHz, DMSO) δ 9.52 (s, 1H), 8.54 (s, 1H), 8.27 (s, 1H), 8.11 (d, J=8.6 Hz, 1H), 7.65 (s, 1H), 7.39 (s, 1H), 6.93 (t, J=4.6 Hz, 1H), 3.90 (s, 1H), 3.73 (s, 1H), 3.08-3.02 (m, 3H). HPLC-MS (ESI+): m/z 391.2 (M+H)⁺.

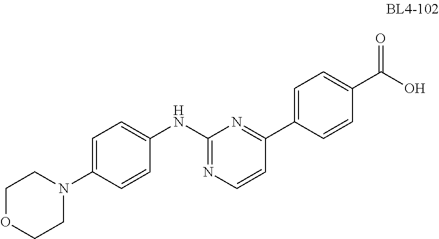 [SMILES: O=C(O)c1ccc(-c2ccnc(Nc3ccc(N4CCOCC4)cc3)n2)cc1]

4-(2-((4-Morpholinophenyl)amino)pyrimidin-4-yl)benzoic acid (BL4-102) 2: To a 20 mL microwave vial was added BL4-101, LiOH (96 mg, 4.0 mmol, 10.0 eq.), THF (9 mL) and H₂O (3 mL). The mixture was heated to 70° C. for 4 h. The reaction mixture was gradually cooled down to ambient temperature before being quenched with saturated H₂O (10 mL). The residue was washed with Et₂O (10 mL), and the aqueous layer was acidified by 1M HCl to pH˜4, followed by being extracted with ethyl acetate (3×15 mL). The combined organic layers were washed with brine (10 mL), dried (Na₂SO₄) and filtered. The residue was concentrated under reduced pressure to give BL4-102, which was used for next step without further purification. ¹H NMR (500 MHz, DMSO-d6) δ 9.50 (s, 1H), 8.54 (d, J=5.1 Hz, 1H), 8.25 (d, J=8.6 Hz, 2H), 8.08 (d, J=8.4 Hz, 2H), 7.67 (d, J=9.0 Hz, 2H), 7.38 (d, J=5.2 Hz, 1H), 6.94 (d, J=9.1 Hz, 2H), 3.79-3.71 (m, 4H), 3.09-3.01 (m, 4H). HPLC-MS (ESI+): m/z 377.0 (M+H)⁺.

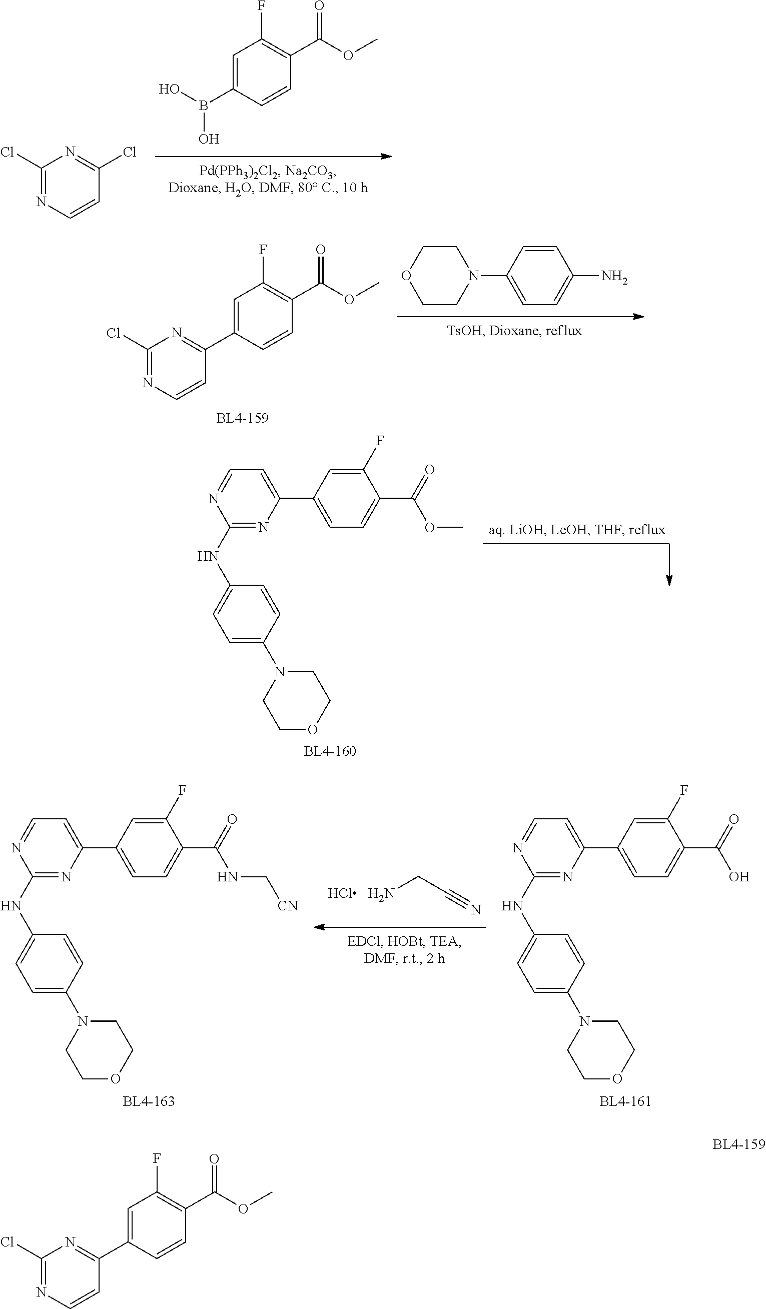

Methyl 4-(2-chloropyrimidin-4-yl)-2-fluorobenzoate (BL4-159): 3 This compound was synthesized by using the same procedure described for BL4-095 except using 2,4-dichloropyrimidine (1 g, 6.71 mmol), (3-fluoro-4-(methoxycarbonyl)phenyl)boronic acid (1.6 g, 8.05 mmol), Pd(PPh₃) Cl₂ (471 mg, 0.67 mmol), and Na₂CO₃ (2.1 g, 20.13 mmol) to give the BL4-159 (1.02 g, 58%) as a offwithe solid. ¹H NMR (500 MHz, DMSO-d6) δ 8.95 (d, J=5.2 Hz, 1H), 8.30 (d, J=5.3 Hz, 1H), 8.19-8.15 (m, 2H), 8.08 (d, J=8.1 Hz, 1H), 3.91 (s, 3H). HPLC-MS (ESI+): m/z 289.1 (M+H)⁺.

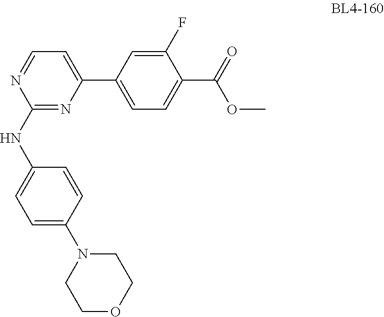 [SMILES: COC(=O)c1ccc(-c2ccnc(Nc3ccc(N4CCOCC4)cc3)n2)cc1F]

Methyl 2-fluoro-4-(2-((4-morpholinophenyl)amino)pyrimidin-4-yl)benzoate (BL4-160): This compound was synthesized by using the same procedure described for BL4-101 except using BL4-159 (200 mg, 0.75 mmol), 4-morpholinoaniline (147 mg, 0.83 mmol), TsOH·H₂O (157 mg, 0.83 mmol), and dioxane (4 mL) to give the BL4-160 (208 mg, 68%) as a yellow solid. ¹H NMR (500 MHz, DMSO-d6) δ 9.56 (s, 1H), 8.58 (d, J=5.0 Hz, 1H), 8.14-8.03 (m, 3H), 7.64 (d, J=9.0 Hz, 2H), 7.45 (d, J=5.0 Hz, 1H), 6.94 (d, J=9.0 Hz, 2H), 3.90 (s, 3H), 3.78-3.71 (m, 4H), 3.11-3.03 (m, 4H). HPLC-MS (ESI+): m/z 409.0 (M+H)⁺.

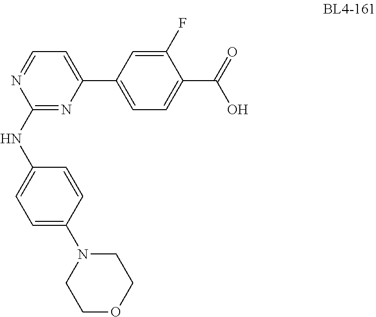 [SMILES: O=C(O)c1ccc(-c2ccnc(Nc3ccc(N4CCOCC4)cc3)n2)cc1F]

2-Fluoro-4-(2-((4-morpholinophenyl)amino)pyrimidin-4-yl)benzoic acid (BL4-161): This compound was synthesized by using the same procedure described for BL4-102 except using BL4-160 (206 mg, 0.5 mmol) and LiOH (121 mg, 5.0 mmol) to give the BL4-161 (120 mg, 61%) as a brown solid. ¹H NMR (500 MHz, DMSO-d6) δ 9.62 (s, 1H), 8.58 (d, J=5.1 Hz, 1H), 8.11-7.98 (m, 3H), 7.68 (d, J=8.5 Hz, 2H), 7.46 (d, J=5.1 Hz, 1H), 7.06 (brs, 2H), 3.83-3.75 (m, 4H), 3.08-3.16 (m, 4H). HPLC-MS (ESI+): m/z 395.0 (M+H)⁺.

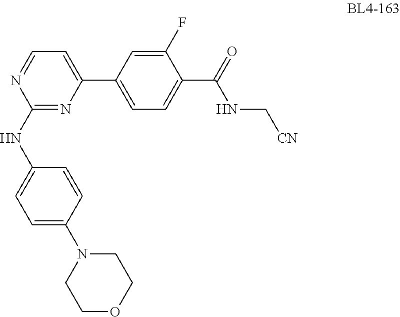 [SMILES: N#CCNC(=O)c1ccc(-c2ccnc(Nc3ccc(N4CCOCC4)cc3)n2)cc1F]

N-(Cyanomethyl)-2-fluoro-4-(2-((4-morpholinophenyl)amino)pyrimidin-4-yl)benzamide (BL4-163): This compound was synthesized by using the same procedure described for BL4-106 except using BL4-161 (50 mg, 0.15 mmol) to give the BL4-163 (35 mg, 64%) as a yellow solid. ¹H NMR (500 MHz, DMSO-d6) δ 9.54 (s, 1H), 9.14 (s, 1H), 8.57 (d, J=5.1 Hz, 1H), 8.12-8.05 (m, 2H), 7.86 (t, J=7.8 Hz, 1H), 7.65 (d, J=9.1 Hz, 2H), 7.44 (d, J=5.2 Hz, 1H), 6.94 (d, J=9.1 Hz, 2H), 4.36 (d, J=4.2 Hz, 2H), 3.80-3.71 (m, 4H), 3.11-3.01 (m, 4H). HPLC 97.3% (t_{R}=14.3 min, Grad CH₃OH 5-95% in H₂O, 0.1 TFA 1 mL, 20 min). HPLC-MS (ESI+): m/z 433.0 (M+H)⁺. HRMS (ESI+) m/z calculated for C₂₃H₂₂FN₆O₂ (M+H)⁺ 433.1783, found 433.1781.

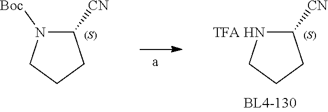

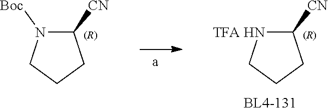

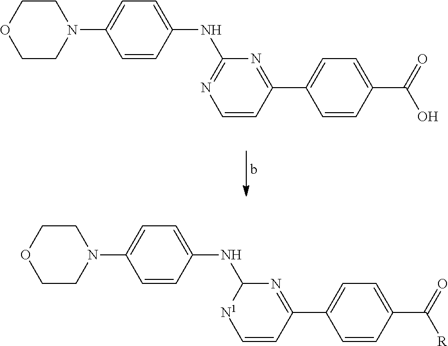

| Compd. | R |
|---|---|
| BL4-142 | 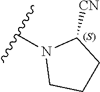 |
| BL4-143 | 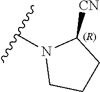 |
| BL4-110 | 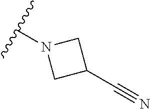 [SMILES: *N1CC(C#N)C1] |
| BL4-153 | 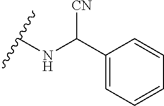 [SMILES: *NC(C#N)c1ccccc1] |
| BL4-157 | 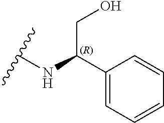 |
| BL4-158 | 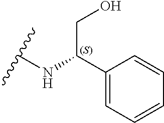 |
| BL4-174 | 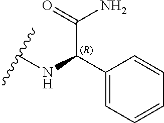 |
| BL4-175 | 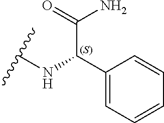 |

Reagents and Conditions: (a) TFA, DCM, 0° C., 30 min, quant.; (b) EDCl, HOBt, Et₃N, DMF, r.t., 4 h: BL4-130, 45% for BL4-142; BL4-131, 56% for BL4-143; azetidine-3-carbonitrile hydrochloride, 64% for BL4-110; 2-phenylglycinonitrile hydrochloride, 45% for BL4-153; (R)-Phenylglycinol, 46% for BL4-157; (S)-Phenylglycinol, 43% for BL4-158. (S)-2-Cyanopyrrolidin-1-ium trifluoroacetate (BLA-130):⁴ This compound was prepared from tert-butyl(S)-2-cyanopyrrolidine-1-carboxylate (392 mg, 2.0 mmol), and TFA (3.6 mL, 50.0 mmol) to give the BL4-130 quantitively. ¹H NMR (500 MHz, DMSO-d6) δ 9.72 (s, 2H), 4.70 (t, J=7.5 Hz, 1H), 3.30-3.22 (m, 2H), 2.35 (dtd, J=13.6, 7.8, 5.8 Hz, 1H), 2.15 (dq, J=13.0, 7.4 Hz, 1H), 2.06-1.91 (m, 2H).

(R)-Pyrrolidine-2-carbonitrile 2,2,2-trifluoroacetaldehyde (BL4-131): This was prepared from tert-butyl (R)-2-cyanopyrrolidine-1-carboxylate (392 mg, 2.0 mmol) using TFA (3.6 mL, 50.0 mmol) to give BL4-131 quantitatively. ¹H NMR (500 MHz, DMSO-d6) δ 9.74 (s, 2H), 4.71 (t, J=7.5 Hz, 1H), 3.30-3.22 (m, 2H), 2.32 (dtd, J=13.6, 7.8, 5.8 Hz, 1H), 2.14 (dq, J=13.0, 7.4 Hz, 1H), 2.05-1.96 (m, 2H).

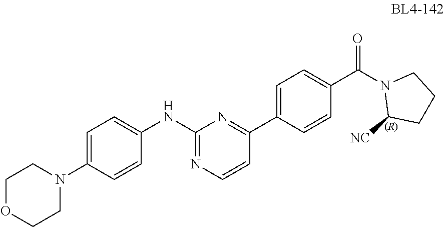

(S)-1-(4-(2-((4-morpholinophenyl)amino)pyrimidin-4-yl)benzoyl)pyrrolidine-2-carbonitrile (BL4-142): This was prepared from BL4-130 (39 mg, 0.2 mmol) using the same procedure described for BL4-106 to give the title compound as a yellow solid (25 mg, 51%). ¹H NMR (500 MHz, DMSO-d6) δ 9.49 (s, 1H), 8.54 (d, J=5.1 Hz, 1H), 8.24 (d, J=8.2 Hz, 2H), 7.81-7.64 (m, 5H), 7.39 (d, J=5.2 Hz, 1H), 6.99-6.89 (m, 2H), 4.92 (ddd, J=8.1, 5.0, 3.1 Hz, 2H), 3.78-3.72 (m, 5H), 3.65 (t, J=6.5 Hz, 1H), 3.52 (d, J=9.4 Hz, 2H), 3.10-2.99 (m, 4H), 2.40-2.31 (m, 1H), 2.23-2.11 (m, 1H), 1.99 (dq, J=15.9, 6.1, 5.1 Hz, 3H). HPLC 95.1% (t_{R}=14.9 min, Grad CH₃OH 5-95% in H₂O, 0.1 TFA 1 mL, 20 min). HPLC-MS (ESI+): m/z 455.1 (M+H)⁺. HRMS (ESI+) m/z calculated for C₂₆H₂₇N₆O₂ (M+H)⁺ 455.2185, found 455.2184.

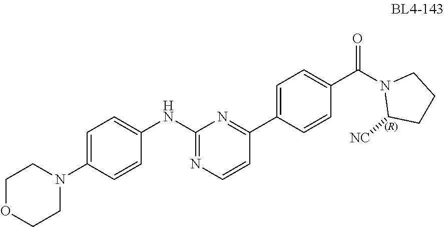

(R)-1-(4-(2-((4-Morpholinophenyl)amino)pyrimidin-4-yl)benzoyl)pyrrolidine-2-carbonitrile (BL4-143): This was prepared from BL4-131 (39 mg, 0.2 mmol) using the same procedure described for BL4-106 to give the title compound as a yellow solid (28 mg, 54%). ¹H NMR (500 MHz, DMSO-d6) δ 9.49 (s, 1H), 8.54 (d, J=5.1 Hz, 1H), 8.24 (d, J=8.2 Hz, 2H), 7.75 (dd, J=17.2, 8.0 Hz, 3H), 7.67 (d, J=8.5 Hz, 2H), 7.39 (d, J=5.2 Hz, 1H), 6.98-6.90 (m, 2H), 4.93-4.89 (m, 1H), 3.78-3.71 (m, 5H), 3.67-3.61 (m, 1H), 3.52 (q, J=9.1, 7.8 Hz, 1H), 3.09-3.02 (m, 4H), 2.37-2.31 (m, 1H), 2.20 (dt, J=12.1, 5.8 Hz, 2H), 1.98 (ddt, J=16.8, 12.1, 6.1 Hz, 3H). HPLC 95.1% (t_{R}=15.1 min, Grad CH₃OH 5-95% in H₂O, 0.1 TFA 1 mL, 20 min). HPLC-MS (ESI+): m/z 455.1 (M+H)⁺. HRMS (ESI+) m/z calculated for C₂₆H₂₇N₆O₂ (M+H)⁺ 455.2190, found 455.2184.

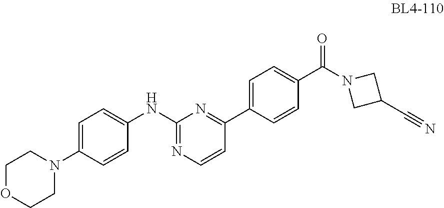 [SMILES: N#CC1CN(C(=O)c2ccc(-c3ccnc(Nc4ccc(N5CCOCC5)cc4)n3)cc2)C1]

1-(4-(2-((4-Morpholinophenyl)amino)pyrimidin-4-yl)benzoyl) azetidine-3-carbonitrile (BL4-110): This was prepared from azetidine-3-carbonitrile HCl (15 mg, 0.13 mmol) using the same procedure described for BL4-106 to give the title compound as a yellow solid (28 mg, 59%). ¹H NMR (500 MHz, DMSO-d6) δ 9.49 (s, 1H), 8.54 (d, J=5.1 Hz, 1H), 8.22 (d, J=8.4 Hz, 2H), 7.80 (d, J=8.5 Hz, 2H), 7.67 (d, J=9.0 Hz, 2H), 7.39 (d, J=5.2 Hz, 1H), 6.94 (d, J=9.1 Hz, 2H), 4.60 (m, 2H), 4.39 (br, 1H), 4.22 (br, 1H), 3.88 (tt, J=9.1, 6.2 Hz, 1H), 3.77-3.70 (m, 4H), 3.11-2.99 (m, 4H). HPLC 98.9% (t_{R}=12.6 min, Grad CH₃OH 5-95% in H₂O, 0.1 TFA 1 mL, 20 min). HPLC-MS (ESI+): m/z 441.0 (M+H)⁺. HRMS (ESI+) m/z calculated for C₂₅H₂₅N₆O₂ (M+H)⁺ 441.2034, found 441.2027.

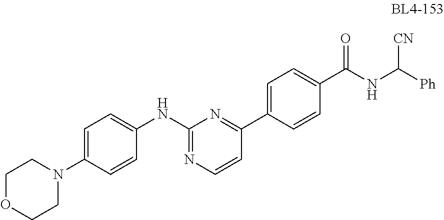 [SMILES: N#CC(NC(=O)c1ccc(-c2ccnc(Nc3ccc(N4CCOCC4)cc3)n2)cc1)c1ccccc1]

N-(Cyano (phenyl)methyl)-4-(2-((4-morpholinophenyl)amino)pyrimidin-4-yl)benzamide (BL4-153): This was prepared from 2-amino-2-phenylacetonitrile HCl (40 mg, 0.24 mmol) using the same procedure described for BL4-106 to give the title compound as a yellow solid (35 mg, 45%). ¹H NMR (500 MHz, DMSO-d6) δ 9.89 (d, J=7.9 Hz, 1H), 9.50 (s, 1H), 8.54 (d, J=5.1 Hz, 1H), 8.26 (d, J=8.5 Hz, 2H), 8.07 (d, J=8.5 Hz, 2H), 7.67 (d, J=9.0 Hz, 2H), 7.57 (d, J=7.4 Hz, 2H), 7.48 (1, J=7.4 Hz, 2H), 7.44-7.38 (m, 2H), 6.93 (d, J=9.1 Hz, 2H), 6.45 (d, J=7.8 Hz, 1H), 3.78-3.71 (m, 4H), 3.09-3.01 (m, 4H). HPLC 96.4% (t_{R}=16.6 min, Grad CH₃OH 5-95% in H₂O, 0.1 TFA 1 mL, 20 min). HPLC-MS (ESI+): m/z 491.0 (M+H)⁺. HRMS (ESI+) m/z calculated for C₂₉H₂₇N₆O₂ (M+H)⁺ 491.2190, found 491.2168.

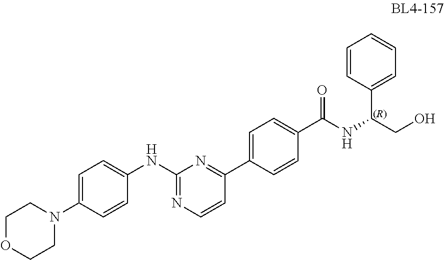

(R)—N-(2-Hydroxy-1-phenylethyl)-4-(2-((4-morpholinophenyl)amino)pyrimidin-4-yl)benzamide (BL4-157): This was prepared from (R)-2-amino-2-phenylethan-1-ol (20 mg, 0.15 mmol) using the same procedure described for BL4-106 to give the title compound as a yellow solid (20 mg, 41%). ¹H NMR (500 MHz, DMSO-d6) δ 9.49 (s, 1H), 8.85 (d, J=8.1 Hz, 1H), 8.54 (d, J=5.1 Hz, 1H), 8.25 (d, J=8.4 Hz, 2H), 8.07 (d, J=8.4 Hz, 2H), 7.68 (d, J=9.0 Hz, 2H), 7.42 (t, J=6.4 Hz, 3H), 7.34 (t, J=7.6 Hz, 2H), 7.25 (t, J=7.3 Hz, 1H), 6.94 (d, J=9.0 Hz, 2H), 5.11 (q, J=8.0 Hz, 1H), 4.98 (t, J=5.8 Hz, 1H), 3.75 (dd, J=6.0, 3.6 Hz, 5H), 3.68 (dt, J=11.1, 5.6 Hz, 1H), 3.09-3.03 (m, 4H). HPLC 95.5% (t_{R}=15.7 min, Grad CH₃OH 5-95% in H₂O, 0.1 TFA 1 mL, 20 min). HPLC-MS (ESI+): m/z 496.1 (M+H)⁺. HRMS (ESI+) m/z calculated for C₂₉H₂₇N₆O₂ (M+H)⁺ 496.2343, found 496.2328.

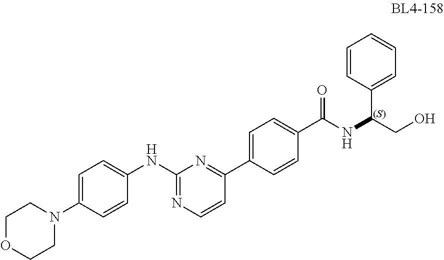

(S)—N-(2-Hydroxy-1-phenylethyl)-4-(2-((4-morpholinophenyl)amino)pyrimidin-4-yl)benzamide (BL4-158): This was prepared from(S)-2-amino-2-phenylethan-1-ol (20 mg, 0.15 mmol) using the same procedure described for BL4-106 to give the title compound as a yellow solid (23 mg, 42%). ¹H NMR (500 MHz, DMSO-d6) δ 9.49 (s, 1H), 8.85 (d, J=8.1 Hz, 1H), 8.54 (d, J=5.2 Hz, 1H), 8.25 (d, J=8.1 Hz, 2H), 8.07 (d, J=8.1 Hz, 2H), 7.68 (s, 1H), 7.42 (t, J=6.7 Hz, 3H), 7.33 (d, J=7.6 Hz, 2H), 7.25 (t, J=7.3 Hz, 1H), 6.94 (d, J=8.9 Hz, 2H), 5.11 (q, J=7.9 Hz, 1H), 4.98 (t, J=5.6 Hz, 1H), 3.75 (dd, J=6.0, 3.6 Hz, 5H), 3.67 (dd, J=11.1, 5.5 Hz, 1H), 3.06 (t, J=4.8 Hz, 4H). HPLC 96.7% (t_{R}=15.7 min, Grad CH₃OH 5-95% in H₂O, 0.1 TFA 1 mL, 20 min). HPLC-MS (ESI+): m/z 496.1 (M+H)⁺. HRMS (ESI+) m/z calculated for C₂₉H₂₇N₆O₂ (M+H)⁺ 496.2343, found 496.2326.

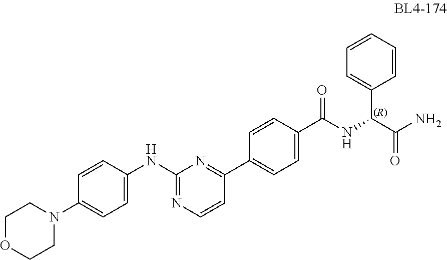

(R)—N-(2-amino-2-oxo-1-phenylethyl)-4-(2-((4-morpholinophenyl)amino)pyrimidin-4-yl)benzamide (BL4-174): This was prepared from (R)-2-amino-2-phenylacetamide (22 mg, 0.15 mmol) using the same procedure described for BL4-106 to give the title compound as a yellow solid (28 mg, 50%). ¹H NMR (600 MHz, DMSO-d6) δ 9.55 (s, 1H), 8.92 (d, J=7.9 Hz, 1H), 8.59 (d, J=5.2 Hz, 1H), 8.29 (d, J=8.5 Hz, 2H), 8.13 (d, J=8.5 Hz, 2H), 7.79 (s, 1H), 7.73 (d, J=9.0 Hz, 2H), 7.61 (d, J=7.4 Hz, 2H), 7.47-7.40 (m, 3H), 7.38 (t, J=6.8 Hz, 1H), 7.32 (s, 1H), 7.01 (s, 2H), 5.71 (d, J=7.9 Hz, 1H), 3.86-3.75 (m, 4H), 3.13-3.05 (m, 4H). HPLC 95.1% (t_{R}=15.4 min, Grad CH₃OH 5-95% in H₂O, 0.1 TFA 1 mL, 20 min). HPLC-MS (ESI+): m/z 509.1 (M+H)⁺. HRMS (ESI+) m/z calculated for C₂₉H₂₉N₆O₃ (M+H)⁺ 509.2296, found 509.2293.

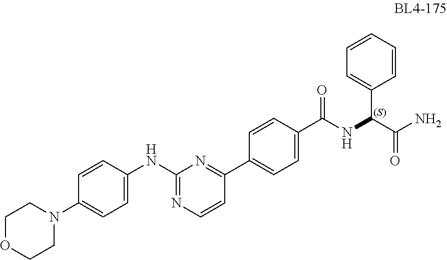

(S)—N-(2-amino-2-oxo-1-phenylethyl)-4-(2-((4-morpholinophenyl)amino)pyrimidin-4-yl)benzamide (BL4-175): This was prepared from(S)-2-amino-2-phenylacetamide (22 mg, 0.15 mmol) using the same procedure described for BL4-106 to give the title compound as a yellow solid (22 mg, 41%). ¹H NMR (600 MHz, DMSO-d6) δ 9.49 (s, 1H), 8.87 (d, J=7.9 Hz, 1H), 8.53 (d, J=5.1 Hz, 1H), 8.23 (d, J=8.5 Hz, 2H), 8.07 (d, J=8.5 Hz, 2H), 7.74-7.63 (m, 3H), 7.55 (d, J=7.4 Hz, 2H), 7.40-7.34 (m, 3H), 7.32 (t, J=7.3 Hz, 1H), 7.26 (s, 1H), 6.94 (d, J=9.1 Hz, 2H), 5.66 (d, J=7.9 Hz, 1H), 3.75 (m, 4H), 3.10-3.00 (m, 4H). HPLC 95.1% (t_{R}=15.4 min, Grad CH₃OH 5-95% in H₂O, 0.1 TFA 1 mL, 20 min). HPLC-MS (ESI+): m/z 509.1 (M+H)⁺. HRMS (ESI+) m/z calculated for C₂₉H₂₉N₆O₃ (M+H)⁺ 509.2296, found 509.2290.

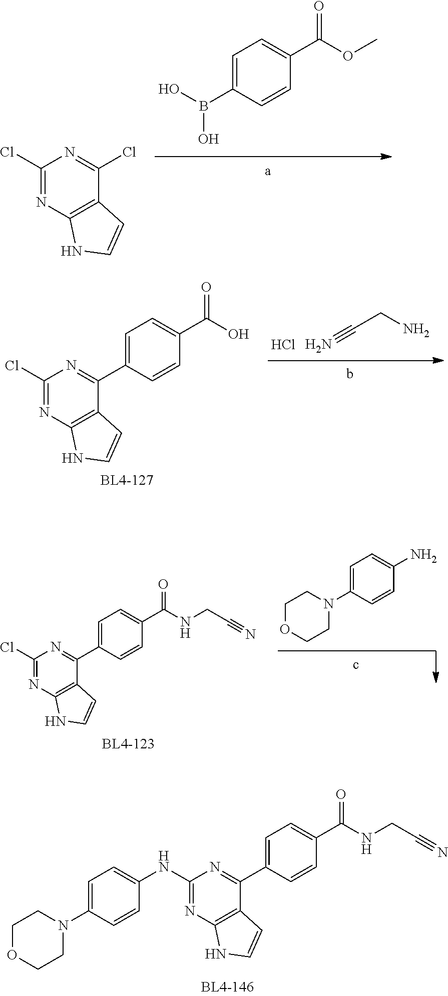

Reagents and Conditions: (a) Pd(PPh₃)₄, Na₂CO₃, dioxane, argon, 150° C., overnight, 52%; (b) EDCl, HOBt, Et₃N, DMF, r.t., 4 h, 61%; (c) Pd₂(dba)₃, XPhos, K₂CO₃, t-BuOH, argon, 100° C., 4 h, 21%.

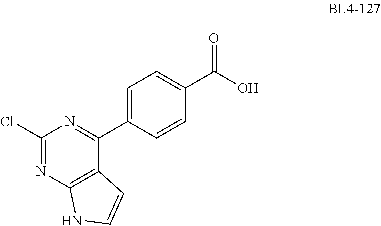 [SMILES: O=C(O)c1ccc(-c2nc(Cl)nc3[nH]ccc23)cc1]

4-(2-Chloro-7H-pyrrolo[2,3-d]pyrimidin-4-yl)benzoic acid (BL4-127): To a 150 mL pressure vessel were added 2,4-dichloro-7H-pyrrolo[2,3-d]pyrimidine (2 g, 10.6 mmol, 1.0 eq.), (4-(methoxycarbonyl)phenyl)boronic acid (1.9 g, 10.6 mmol, 1.0 eq.), Na₂CO₃ (15.9 mL, 2 M in H₂O, 3.0 eq.), and 1,4-dioxane (60 mL). The mixture was degassed by bubbling argon for 5 minutes before Pd(PPh₃)₄ (367 mg, 0.32 mmol, 3% mmol) was added. The mixture was heated to 150° C. overnight. The reaction mixture was gradually cooled down to ambient temperature before being quenched with H₂O (100 mL). The residue was washed with Et₂O (50 mL), and the aqueous layer was acidified with 1M HCl to pH˜4, followed by being extracted with ethyl acetate (3×75 mL). The combined organic layers were washed with brine (50 mL), dried (Na₂SO₄) and filtered. The residue was concentrated under reduced pressure, and the residue was purified by column chromatography (ethyl acetate 0 to 60% in hexanes) to give BL4-127 (1.5 g, 52%) as a yellow solid. ¹H NMR (500 MHz, DMSO-d6) δ 12.57 (s, 1H), 8.27 (d, J=8.4 Hz, 2H), 8.14 (d, J=8.4 Hz, 2H), 7.76 (dd, J=3.3, 1.7 Hz, 1H), 7.00 (d, J=3.1 Hz, 1H). HPLC-MS (ESI+): m/z 273.9 (M+H)⁺.

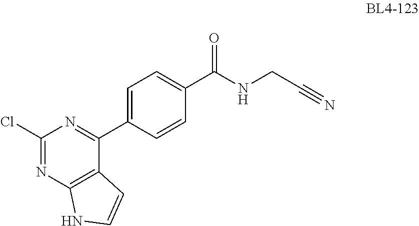 [SMILES: N#CCNC(=O)c1ccc(-c2nc(Cl)nc3[nH]ccc23)cc1]

4-(2-Chloro-7H-pyrrolo[2,3-d]pyrimidin-4-yl)-N-(cyanomethyl)benzamide (BL4-123): This compound was prepared using the same procedure described for BL4-106 from BL4-127 (55 mg, 0.2 mmol) to give the title compound (30 mg, 48%) as a yellow solid. ¹H NMR (500 MHz, DMSO-d6) δ 9.40 (t, J=5.5 Hz, 1H), 8.28 (d, J=8.5 Hz, 2H), 8.08 (d, J=8.5 Hz, 2H), 7.76 (d, J=3.6 Hz, 1H), 7.00 (d, J=3.6 Hz, 1H), 4.36 (d, J=5.4 Hz, 2H). HPLC-MS (ESI+): m/z 311.9 (M+H)⁺.

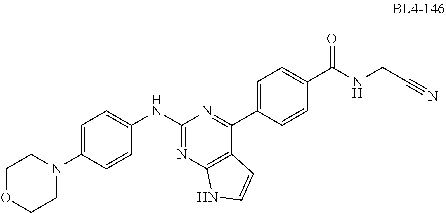 [SMILES: N#CCNC(=O)c1ccc(-c2nc(Nc3ccc(N4CCOCC4)cc3)nc3[nH]ccc23)cc1]

N-(Cyanomethyl)-4-(2-((4-morpholinophenyl)amino)-7H-pyrrolo[2,3-d]pyrimidin-4-yl)benzamide (BL4-146): A mixture of BL4-123 (20 mg, 0.06 mmol, 1.0 eq.), 4-morpholinoaniline (11.4 mg, 0.06 mmol, 1.0 eq.) and K₂CO₃ (17 mg, 0.13 mmol, 2.0 eq.) in anhydrous tert-BuOH (0.5 mL) was degassed by bubbling argon for 5 min. XPhos (3 mg, 0.006 mmol, 0.1 eq.) and Pd₂(dba)₃ (3 mg, 0.003 mmol, 0.05 eq.) were added and the mixture was stirred at 100° C. overnight. The mixture was allowed to cool to room temperature and quenched with water (5 mL). The mixture was extracted with ethyl acetate (3×5 mL). The combined organic extracts were washed with brine, dried (Na₂SO₄), filtered and the solvent was removed under reduced pressure. The residue was purified by flash chromatography (0 to 80% EtOAc in Hexanes) to afford BL4-146 (6 mg, 21%). ¹H NMR (500 MHz, DMSO-d6) δ 11.67 (s, 1H), 9.35 (t, J=5.5 Hz, 1H), 9.18 (s, 1H), 8.25 (d. J=8.4 Hz, 2H), 8.06 (d, J=8.4 Hz, 2H), 7.76 (d, J=7.6 Hz, 2H), 7.28 (br, 1H), 6.95 (br, 2H), 6.70 (br, 1H), 4.36 (d, J=5.4 Hz, 2H), 3.81-2.77 (m, 4H), 3.08-2.98 (m, 4H). HPLC 96.3% (t_{R}=13.5 min, Grad CH₃OH 5-95% in H₂O, 0.1 TFA 1 mL, 20 min). HPLC-MS (ESI+): m/z 454.0 (M+H)⁺. HRMS (ESI+) m/z calculated for C₂₅H₂₄N₇O₂ (M+H)⁺ 454.1986, found 454.1978.

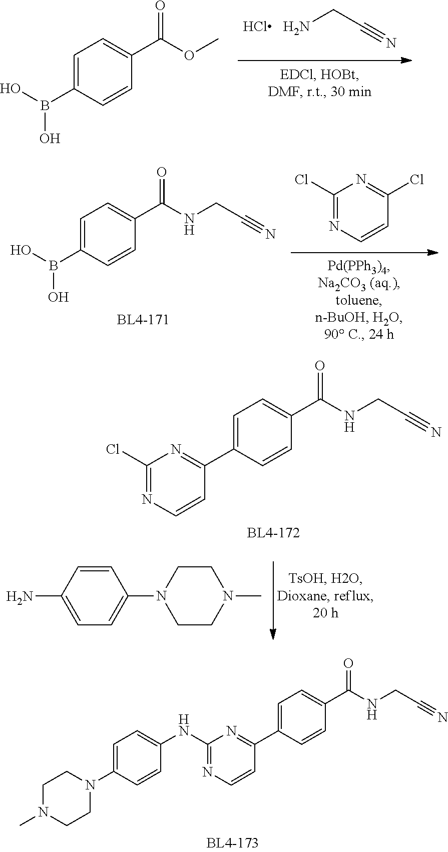

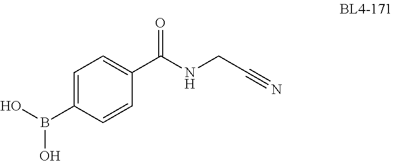 [SMILES: N#CCNC(=O)c1ccc(B(O)O)cc1]

(4-((Cyanomethyl)carbamoyl)phenyl)boronic acid (BL4-171): 5 This compound was prepared using the same procedure described for BL4-106 from 4-boronobenzoic acid (100 mg, 0.6 mmol), 2-aminoacetonitrile hydrogen chloride (84 mg, 0.9 mmol, 1.5 eq.), EDC. HCl (364 mg, 1.9 mmol, 3.0 eq.), HOBt (122 mg, 0.9 mmol, 1.5 eq.), and TEA (294 μL, 2.11 mmol, 3.5 eq.) to give the title compound as an off white solid (92 mg, 75%). ¹H NMR (500 MHz, DMSO-d6) δ 9.19 (s, 1H), 7.87 (d, J=8.1 Hz, 2H), 7.81 (d, J=8.1 Hz, 2H), 4.31 (d, J=4.7 Hz, 2H). HPLC-MS (ESI+): m/z 203.1 (M−H)⁺.

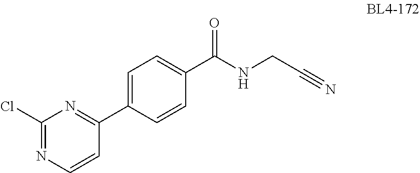 [SMILES: N#CCNC(=O)c1ccc(-c2ccnc(Cl)n2)cc1]

4-(2-Chloropyrimidin-4-yl)-N-(cyanomethyl)benzamide (BL4-172): 2 To a 20 mL microwave vial was added BL4-171 (90 mg, 0.44 mmol, 1.0 eq.), 2,4-dichloropyrimidine (79 mg, 0.53 mmol, 1.2 eq.), Na₂CO₃ (440 μl, 2 M in H₂O, 2.0 eq.), toluene (3.5 mL), and n-BuOH (1.0 mL). The mixture was degassed by bubbling argon for 5 minutes before being treated with Pd(PPh₃)₄ (25 mg, 0.02 mmol, 5% mmol). The mixture was heated to 90° C. under argon for 1 day. The reaction mixture was gradually cooled down to ambient temperature before being quenched with H₂O (10 mL). The residue was diluted with ethyl acetate (20 mL) and two layers were separated, and the aqueous layer was further extracted with ethyl acetate (3×15 mL). The combined organic layers were washed with brine (10 mL), dried (Na₂SO₄), filtered, and concentrated under reduced pressure. The residue purified by column chromatography (ethyl acetate 0 to 60% in hexanes) to give the title compound as a white solid (41 mg, 36%). ¹H NMR (500 MHz, DMSO-d6) δ 9.40 (t, J=5.4 Hz, 1H), 8.89 (d, J=5.3 Hz, 1H), 8.33 (d, J=8.4 Hz, 2H), 8.24 (d, J=5.3 Hz, 1H), 8.05 (d, J=8.4 Hz, 2H), 4.35 (d, J=5.4 Hz, 2H). HPLC-MS (ESI+): m/z 272.9 (M+H)⁺.

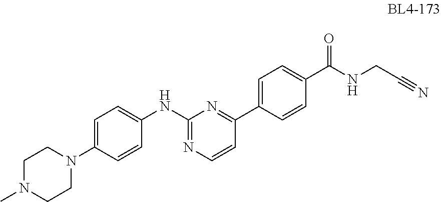 [SMILES: CN1CCN(c2ccc(Nc3nccc(-c4ccc(C(=O)NCC#N)cc4)n3)cc2)CC1]

N-(Cyanomethyl)-4-(2-((4-(4-methylpiperazin-1-yl)phenyl) amino)pyrimidin-4-yl)benzamide (BL4-173): 6 This compound was prepared using the same procedure described for BL4-101 from BL4-172 (40 mg, 0.17 mmol), 4-(4-methylpiperazin-1-yl) aniline (35 mg, 0.18 mmol), TsOH·H₂O, and dioxane (1 mL) to give BL4-173 (28 mg, 39%) as a red solid. ¹H NMR (600 MHz, DMSO-d6) δ 9.48 (s, 1H), 9.34 (t, J=5.3 Hz, 1H), 8.54 (d, J=5.1 Hz, 1H), 8.27 (d, J=8.5 Hz, 2H), 8.03 (d, J=8.5 Hz, 2H), 7.65 (d, J=9.0 Hz, 2H), 7.40 (d, J=5.2 Hz, 1H), 6.93 (d, J=9.1 Hz, 2H), 6.71-6.65 (m, 1H), 6.54-6.45 (m, 1H), 4.36 (d, J=5.4 Hz, 2H), 3.12-3.05 (m, 4H), 2.48-2.45 (m, 4H), 2.23 (s, 3H). HPLC 95.6% (t_{R}=13.3 min, Grad CH₃OH 5-95% in H₂O, 0.1 TFA 1 mL, 20 min). HPLC-MS (ESI+): m/z 428.0 (M+H)⁺. HRMS (ESI+) m/z calculated for C₂₄H₂₆N₇O (M+H)⁺ 428.2188, found 428.2183.

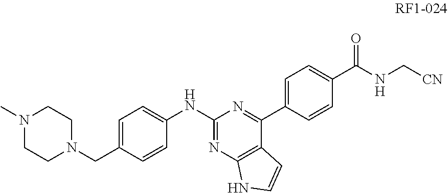 [SMILES: CN1CCN(Cc2ccc(Nc3nc(-c4ccc(C(=O)NCC#N)cc4)c4cc[nH]c4n3)cc2)CC1]

N-(Cyanomethyl)-4-(2-((4-((4-methylpiperazin-1-yl)methyl) phenyl)amino)-7H-pyrrolo[2,3-d]pyrimidin-4-yl)benzamide (RF1-024): A 10-mL pressure vessel, dried and under argon atmosphere, was charged with 4-(2-chloro-7H-pyrrolo[2,3-d]pyrimidin-4-yl)-N-(cyano-methyl)benzamide BL4-123 (100 mg, 0.321 mmol, 1 equiv), 4,4-methylpiperazin-1-ylmethyl)phenylamine 4-[(4-methylpiperazin-1-yl)methyl]aniline (65.9 mg, 0.321 mmol, 1 equiv), Pd₂(dba)₃ (29.2 mg, 0.0321 mmol, 10 mol %), XPhos (30.6 mg, 0.0642 mmol, 20 mol %), and K₂CO₃ (88.7 mg, 0.642 mmol, 2 equiv). Finally, t-BuOH (3.0 mL) was added, and the suspension was degassed with argon for 15 min. The vessel was sealed, and the reaction mixture was heated to 100° C. under stirring. After 16 h, the reaction mixture was cooled to room temperature, diluted with water (50 mL) and extracted with EtOAc (5×30 mL). The combined organic phases were washed with brine, dried over MgSO₄, filtered, and concentrated. The crude product was purified by column chromatography (SiO₂, 0-20% MeOH in DCM) to afford RF1-024 (59.0 mg, 0.123 mmol, 38% yield) as a yellow solid. HPLC 99% (t_{R}=7.46 min, 35% CH₃OH in water (0.1% TFA), 1 mL/min, 20 min). ¹H NMR (500 MHz, DMSO-d6) δ 11.71 (brs, 1H), 9.40-9.34 (m, 2H), 8.26 (d, J=8.3 Hz, 2H), 8.07 (d, J=8.5 Hz, 2H), 7.83 (d, J=8.5 Hz, 2H), 7.32 (dd, J=3.6, 2.2 Hz, 1H), 7.18 (d, J=8.4 Hz, 2H), 6.72 (dd, J=3.9, 1.7 Hz, 1H), 4.36 (d, J=5.4 Hz, 2H), 3.41 (s, 2H), 2.39 (brs, 8H), 2.20 (s, 3H). ¹³C NMR (126 MHz, DMSO-d6) δ 166.3, 155.9, 155.4, 154.4, 141.3, 140.4, 133.8, 129.2, 128.5, 127.8, 124.8, 118.0, 117.7, 109.1, 100.2, 61.7, 54.5, 52.1, 45.4, 27.8. HPLC-MS (ESI+): m/z 481.1 (M+H)⁺. HRMS (ESI+) m/z calculated for C₂₇H₂₉N₈O⁺ (M+H)⁺ 481.2459, found 481.2455.

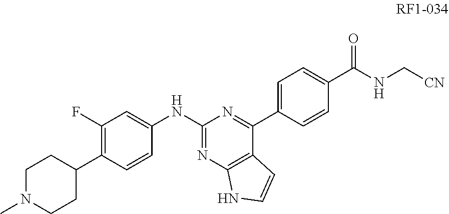 [SMILES: CN1CCC(c2ccc(Nc3nc(-c4ccc(C(=O)NCC#N)cc4)c4cc[nH]c4n3)cc2F)CC1]

N-(Cyanomethyl)-4-(2-((3-fluoro-4-(1-methylpiperidin-4-yl)phenyl)amino)-7H-pyrrolo[2,3-d]pyrimidin-4-yl)benzamide (RF1-034): A 10-mL pressure vessel, dried and under argon atmosphere, was charged with 4-(2-chloro-7H-pyrrolo[2,3-d]pyrimidin-4-yl)-N-(cyano-methyl)benzamide BL4-123 (100 mg, 0.321 mmol, 1 equiv), 3-fluoro-4-(1-methylpiperidin-4-yl) aniline (66.8 mg, 0.321 mmol, 1 equiv), Pd₂(dba); (29.2 mg, 0.0321 mmol, 10 mol %), XPhos (30.6 mg, 0.0642 mmol, 20 mol %), and K₂CO₃ (88.7 mg, 0.642 mmol, 2 equiv). Finally, t-BuOH (3.0 mL) was added, and the suspension was degassed with argon for 15 min. The vessel was sealed, and the reaction mixture was heated to 100° C. under stirring. After 16 h, the reaction mixture was cooled to room temperature, diluted with water (50 mL) and extracted with EtOAc (5×30 mL). The combined organic phases were washed with brine, dried over MgSO₄, filtered, and concentrated. The crude product was purified by column chromatography (SiO₂, 0-20% MeOH in DCM) to afford the product (50.9 mg, 0.105 mmol, 33% yield) as a yellow solid. HPLC 97% (t_{R}=6.89 min, 45% CH₃OH in water (0.1% TFA), 1 mL/min, 20 min). ¹H NMR (500 MHz, DMSO-d6) δ 11.80 (brs, 1H), 9.57 (s, 1H), 9.37 (t, J=5.5 Hz, 1H), 8.26 (d, J=8.4 Hz, 2H), 8.07 (d, J=8.4 Hz, 2H), 7.95 (dd, J=14.0, 2.2 Hz, 1H), 7.50 (dd, J=8.4, 2.2 Hz, 1H), 7.35 (dd, J=3.6, 2.2 Hz, 1H), 7.19 (t, J=8.7 Hz, 1H), 6.74 (dd, J=3.8, 1.7 Hz, 1H), 4.36 (d, J=5.5 Hz, 2H), 2.90-2.81 (m, 2H), 2.71-2.61 (m, 1H), 2.19 (s, 3H), 1.96 (td, J=11.3, 3.6 Hz, 2H), 1.77-1.69 (m, 2H), 1.72-1.62 (m, 2H). ¹⁹F NMR (471 MHz, DMSO-d6) δ −118.99 (dd, J=14.0, 9.1 Hz). ¹³C NMR (126 MHz, DMSO-d6) δ 166.3, 161.0, 159.1, 155.5, 154.9 (d, ¹J_{C-F}=175.3 Hz), 141.2, 141.1 (d, ³J_{C-F}=11.8 Hz), 133.8, 128.6, 127.8, 127.4 (d, ³J_{C-F}=6.8 Hz), 125.0, 123.7 (d, ²J_{C-F}=15.3 Hz), 117.7, 114.1 (d, ⁴J_{C-F}=2.5 Hz), 109.3, 104.6 (d, ²J_{C-F}=28.3 Hz), 100.2, 55.9, 46.3, 34.2, 31.9, 27.8. HPLC-MS (ESI+): m/z 484.1 (M+H)⁺. HRMS (ESI+) m/z calculated for C₂₇H₂₇FN₇O⁺ (M+H)⁺ 484.2256, found 484.2252.

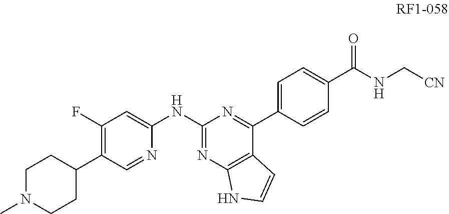 [SMILES: CN1CCC(c2cnc(Nc3nc(-c4ccc(C(=O)NCC#N)cc4)c4cc[nH]c4n3)cc2F)CC1]

N-(Cyanomethyl)-4-(2-((4-fluoro-5-(1-methylpiperidin-4-yl)pyridin-2-yl)amino)-7H-pyrrolo-[2,3-d]pyrimidin-4-yl)benzamide (RF1-058): A 10-mL pressure vessel, dried and under argon atmosphere, was charged with 4-(2-chloro-7H-pyrrolo[2,3-d]pyrimidin-4-yl)-N-(cyano-methyl)benzamide BL4-123 (100 mg, 0.321 mmol, 1 equiv), 4-fluoro-5-(1-methylpiperidin-4-yl)pyridin-2-amine (67.1 mg, 0.321 mmol, 1 equiv), Pd₂(dba)₃ (29.4 mg, 0.0321 mmol, 10 mol %), XPhos (30.6 mg, 0.0642 mmol, 20 mol %), and K₂CO₃ (88.7 mg, 0.642 mmol, 2 equiv). Finally, t-BuOH (3.0 mL) was added, and the suspension was degassed with argon for 15 min. The vessel was sealed, and the reaction mixture was heated to 100° C. under stirring. After 16 h, the reaction mixture was cooled down to rt and poured into water (50 mL). The suspension was filtered and the solid was washed with EtOAc and concentrated under vacuum to afford the product (86.6 mg, 0.179 mmol, 56% yield) as a dark yellow solid. HPLC 94% (t_{R}=12.7 min, CH₃OH 20-95% in water (0.1% TFA), 1 mL/min, 20 min). ¹H NMR (500 MHz, DMSO-d6) δ 11.95 (brs, 1H), 9.76 (s, 1H), 9.38 (t, J=5.5 Hz, 1H), 8.34 (d, J=14.0 Hz, 1H), 8.29 (d, J=8.3 Hz, 2H), 8.21 (d, J=10.9 Hz, 1H), 8.08 (d, J=8.3 Hz, 2H), 7.43 (dd, J=3.6, 2.3 Hz, 1H), 6.80 (dd, J=3.7, 1.8 Hz, 1H), 4.37 (d, J=5.4 Hz, 2H), 3.00-2.93 (m, 2H), 2.75-2.65 (m, 1H), 2.28 (s, 3H), 2.15-2.10 (m, 2H), 1.89-1.73 (m, 4H). ¹⁹F NMR (471 MHz, DMSO-d6) δ −108.54 (t, J=12.7 Hz). HPLC-MS (ESI+): m/z 485.0 (M+H)⁺. HRMS (ESI+) m/z calculated for C₂₆H₂₆FN₈O⁺ (M+H)⁺ 485.2208, found 485.2210.

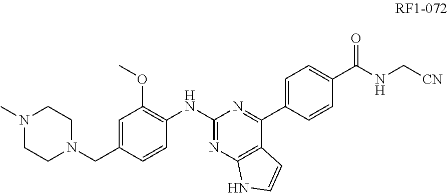 [SMILES: COc1cc(CN2CCN(C)CC2)ccc1Nc1nc(-c2ccc(C(=O)NCC#N)cc2)c2cc[nH]c2n1]

N-(Cyanomethyl)-4-(2-((2-methoxy-4-((4-methylpiperazin-1-yl)methyl) phenyl)amino)-7H-pyrrolo[2,3-d]pyrimidin-4-yl)benzamide (RF1-072): A 10-mL pressure vessel, dried and under argon atmosphere, was charged with 4-(2-chloro-7H-pyrrolo[2,3-d]pyrimidin-4-yl)-N-(cyano-methyl)benzamide BL4-123 (100 mg, 0.321 mmol, 1 equiv), 2-methoxy-4-((4-methylpiperazin-1-yl)methyl) aniline (75.5 mg, 0.321 mmol, 1 equiv), Pd₂(dba)₃ (29.2 mg, 0.0321 mmol, 10 mol %), XPhos (30.6 mg, 0.0642 mmol, 20 mol %), and K₂CO₃ (88.7 mg, 0.642 mmol, 2 equiv). Finally, t-BuOH (3.0 mL) was added, and the suspension was degassed with argon for 15 min. The vessel was sealed, and the reaction mixture was heated to 100° C. under stirring. After 16 h, the reaction mixture was cooled to rt, diluted with water (50 mL) and extracted with EtOAc (5×30 mL). The combined organic phases were washed with brine, dried over MgSO₄, filtered, and concentrated. The crude product was purified by column chromatography (SiO₂, 0-20% MeOH in DCM) to afford the product (51.1 mg, 0.100 mmol, 31% yield) as a yellow solid. HPLC 96% (t_{R}=12.6 min, CH₃OH 20-95% in water (0.1% TFA), 1 mL/min, 20 min). ¹H NMR (500 MHz, DMSO-d6) δ 11.79 (s, 1H), 9.37 (t, J=5.5 Hz, 1H), 8.44 (d, J=8.1 Hz, 1H), 8.27 (d, J=8.4 Hz, 2H), 8.07 (d, J=8.5 Hz, 2H), 7.79 (s, 1H), 7.34 (dd, J=3.6, 2.3 Hz, 1H), 6.97 (d, J=1.7 Hz, 1H), 6.89 (dd, J=8.1, 1.7 Hz, 1H), 6.75 (dd, J=3.6, 1.8 Hz, 1H), 4.37 (d, J=5.4 Hz, 2H), 3.90 (s, 3H), 3.43 (s, 2H), 2.45-2.25 (m, 8H), 2.16 (s, 3H). HPLC-MS (ESI+): m/z 511.0 (M+H)⁺. HRMS (ESI+) m/z calculated for C₂₈H₃₁N₈O₂+ (M+H)⁺ 511.2564, found 511.2557.

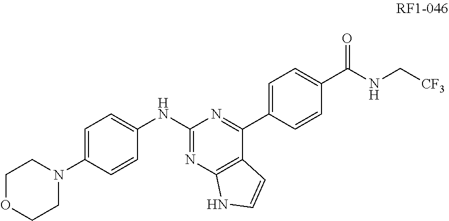 [SMILES: O=C(NCC(F)(F)F)c1ccc(-c2nc(Nc3ccc(N4CCOCC4)cc3)nc3[nH]ccc23)cc1]

4-(2-((4-Morpholinophenyl)amino)-7H-pyrrolo[2,3-d]pyrimidin-4-yl)-N-(2,2,2-trifluoro-ethyl)benzamide (RF1-046): A 10-mL pressure vessel, dried and under argon atmosphere, was charged with 4-(2-chloro-7H-pyrrolo[2,3-d]pyrimidin-4-yl)-N-(2,2,2-trifluoroethyl)benzamide (prepared in a similar way to BH1-103) (100 mg, 0.282 mmol, 1 equiv), 4-morpholinoaniline (50.3 mg, 0.282 mmol, 1 equiv), Pd₂(dba)₃ (25.8 mg, 0.0282 mmol, 10 mol %), XPhos (26.9 mg, 0.0564 mmol, 20 mol %), and K₂CO₃ (77.9 mg, 0.564 mmol, 2 equiv). Finally, t-BuOH (3.0 mL) was added, and the suspension was degassed with argon for 15 min. The vessel was sealed, and the reaction mixture was heated to 100° C. under stirring. After 16 h, the reaction mixture was cooled to rt, diluted with water (50 mL) and extracted with EtOAc (5×30 mL). The combined organic phases were washed with brine, dried over MgSO₄, filtered, and concentrated. The crude product was purified by column chromatography (SiO₂, 0-20% MeOH in DCM) to afford the product (54.4 mg, 0.110 mmol, 39% yield) as a dark yellow solid. HPLC 99% (t_{R}=16.38 min, CH₃OH 20-95% in water (0.1% TFA), 1 mL/min, 20 min). ¹H NMR (500 MHz, DMSO-d6) § 11.63 (brs, 1H), 9.24 (t, J=6.3 Hz, 1H), 9.12 (s, 1H), 8.24 (d, J=8.5 Hz, 2H), 8.08 (d, J=8.5 Hz, 2H), 7.74 (d, J=9.0 Hz, 2H), 7.27 (dd, J=3.6, 2.2 Hz, 1H), 6.90 (d, J=9.1 Hz, 2H), 6.69 (dd, J=3.6, 1.7 Hz, 1H), 4.14 (qd, J=9.7, 6.2 Hz, 2H), 3.77-3.72 (m, 4H), 3.06-3.01 (m, 4H). ¹⁹F NMR (471 MHz, DMSO-d6) δ −70.36 (t, ³J_{F-H}=9.6 Hz). ¹³C NMR (126 MHz, DMSO-d6) δ 166.6, 156.3, 155.5, 154.6, 145.6, 141.3, 134.2, 134.1, 128.5, 127.9, 124.9 (q, ¹J_{C-F}=279.7 Hz), 124.4, 119.6, 115.9, 108.7, 100.1, 66.3, 49.6, 40.0 (q, ²J_{C-F}=22.7 Hz). HPLC-MS (ESI+): m/z 497.0 (M+H)⁺. HRMS (ESI+) m/z calculated for C₂₅H₂₄F₃N₆O₂⁺ (M+H)⁺ 497.1907, found 497.1900.

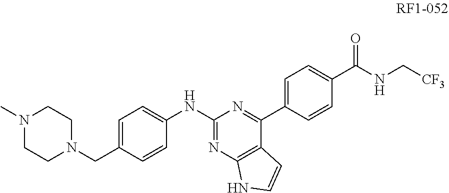 [SMILES: CN1CCN(Cc2ccc(Nc3nc(-c4ccc(C(=O)NCC(F)(F)F)cc4)c4cc[nH]c4n3)cc2)CC1]

4-(2-((4-((4-Methylpiperazin-1-yl)methyl) phenyl)amino)-7H-pyrrolo[2,3-d]pyrimidin-4-yl)-N-(2,2,2-trifluoroethyl)benzamide (RF1-052): A 10-mL pressure vessel, dried and under argon atmosphere, was charged with 4-(2-chloro-7H-pyrrolo[2,3-d]pyrimidin-4-yl)-N-(2,2,2-trifluoroethyl)benzamide (prepared in a similar way to BH1-103) (100 mg, 0.282 mmol, 1 equiv), 4-[(4-methylpiperazin-1-yl)methyl]aniline (57.9 mg, 0.282 mmol, 1 equiv), Pd₂(dba)₃ (25.8 mg, 0.0282 mmol, 10 mol %), XPhos (26.9 mg, 0.0564 mmol, 20 mol %), and K₂CO₃ (77.9 mg, 0.564 mmol, 2 equiv). Finally, t-BuOH (3.0 mL) was added, and the suspension was degassed with argon for 15 min. The vessel was sealed, and the reaction mixture was heated to 100° C. under stirring. After 16 h, the reaction mixture was cooled down to rt, diluted with water (50 mL) and extracted with EtOAc (5×30 mL). The combined organic phases were washed with brine, dried over MgSO₄, filtered, and concentrated. The crude product was purified by column chromatography (SiO₂, 0-20% MeOH in DCM) to afford the product (26.3 mg, 0.0503 mmol, 18% yield) as an orange solid. HPLC 98% (t_{R}=14.9 min, CH₃OH 20-95% in water (0.1% TFA), 1 mL/min, 20 min). ¹H NMR (500 MHz, DMSO-d6) δ 11.72 (brs, 1H), 9.37 (s, 1H), 9.25 (1, J=6.3 Hz, 1H), 8.26 (d, J=8.3 Hz, 2H), 8.09 (d, J=8.3 Hz, 2H), 7.84 (d, J=8.1 Hz, 2H), 7.32 (dd, J=3.6, 2.2 Hz, 1H), 7.19 (d, J=8.3 Hz, 2H), 6.72 (dd, J=3.7, 1.8 Hz, 1H), 4.14 (qd, J=9.7, 6.2 Hz, 2H), 3.42 (s, 2H), 2.41 (brs, 8H), 2.22 (s, 3H). ¹⁹F NMR (471 MHz, DMSO-d6) δ −70.35 (t, ³J_{F-H}=10.0 Hz). ¹³C NMR (126 MHz, DMSO) δ 172.0, 166.6, 155.9, 155.4, 154.4, 141.2, 140.4, 134.2, 129.1, 128.5, 127.9, 124.9 (q, ¹J_{C-F}=279.2 Hz), 124.8, 118.0, 109.1, 100.2, 61.7, 54.5, 52.1, 45.4, 40.1 (q, ²J_{C-F}=24.8 Hz). HPLC-MS (ESI+): m/z 524.0 (M+H)⁺. HRMS (ESI+) m/z calculated for C₂₇H₂₉F₃N₇O⁺ (M+H)⁺ 524.2380, found 524.2377.

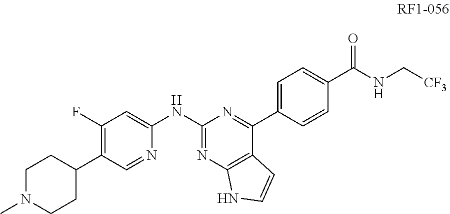 [SMILES: CN1CCC(c2cnc(Nc3nc(-c4ccc(C(=O)NCC(F)(F)F)cc4)c4cc[nH]c4n3)cc2F)CC1]

4-(2-((4-Fluoro-5-(1-methylpiperidin-4-yl)pyridin-2-yl)amino)-7H-pyrrolo[2,3-d]pyrimidin-4-yl)-N-(2,2,2-trifluoroethyl)benzamide (RF1-056): A 10-mL pressure vessel, dried and under argon atmosphere, was charged with 4-(2-chloro-7H-pyrrolo[2,3-d]pyrimidin-4-yl)-N-(2,2,2-trifluoroethyl)benzamide (prepared in a similar way to BH1-103) (100 mg, 0.282 mmol, 1 equiv), 4-fluoro-5-(1-methylpiperidin-4-yl)pyridin-2-amine (59.0 mg, 0.282 mmol, 1 equiv), Pd₂(dba)₃ (25.8 mg, 0.0282 mmol, 10 mol %), XPhos (26.9 mg, 0.0564 mmol, 20 mol %), and K₂CO₃ (77.9 mg, 0.564 mmol, 2 equiv). Finally, t-BuOH (3.0 mL) was added, and the suspension was degassed with argon for 15 min. The vessel was sealed, and the reaction mixture was heated to 100° C. under stirring. After 16 h, the reaction mixture was cooled down to rt, diluted with water (50 mL) and extracted with EtOAc (5×30 mL). The combined organic phases were washed with brine, dried over MgSO₄, filtered, and concentrated. The residue was purified by column chromatography (C18, 5-95% MeOH in water containing 0.1% TFA) to afford RF1-056 (39.6 mg, 0.0752 mmol, 27% yield) as a yellow solid. HPLC 95% (t_{R}=17.2 min, CH₃OH 20-95% in water (0.1% TFA), 1 mL/min, 20 min). ¹H NMR (500 MHz, DMSO) δ 11.81 (brs, 1H), 9.57 (s, 1H), 9.26 (t, J=6.3 Hz, 1H), 8.26 (d, J=8.4 Hz, 2H), 8.09 (d, J=8.4 Hz, 2H), 7.95 (dd, J=14.0, 2.2 Hz, 1H), 7.51 (dd, J=8.5, 2.2 Hz, 1H), 7.35 (dd, J=3.6, 2.2 Hz, 1H), 7.19 (t, J=8.7 Hz, 1H), 6.74 (dd, J=3.7, 1.8 Hz, 1H), 4.14 (qd, J=9.7, 6.2 Hz, 2H), 2.97-2.90 (m, 2H), 2.75-2.65 (m, 1H), 2.26 (s, 3H), 2.09 (td, J=11.4, 3.5 Hz, 2H), 1.80-1.67 (m, 4H). ¹⁹F NMR (471 MHz, DMSO) δ −70.35 (t, ³J_{F-H}=9.6 Hz), —118.93 (dd, J=14.0, 9.0 Hz). HPLC-MS (ESI+): m/z 527.2 (M+H)⁺. HRMS (ESI+) m/z calculated for C₂₇H₂₇F₄N₆O⁺ (M+H)⁺ 527.2177, found 527.2176.

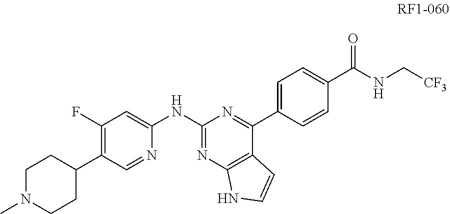 [SMILES: CN1CCC(c2cnc(Nc3nc(-c4ccc(C(=O)NCC(F)(F)F)cc4)c4cc[nH]c4n3)cc2F)CC1]

4-(2-((4-Fluoro-5-(1-methylpiperidin-4-yl)pyridin-2-yl)amino)-7H-pyrrolo[2,3-d]pyrimidin-4-yl)-N-(2,2,2-trifluoroethyl)benzamide (RF1-060): A 10-mL pressure vessel, dried and under argon atmosphere, was charged with 4-(2-chloro-7H-pyrrolo[2,3-d]pyrimidin-4-yl)-N-(2,2,2-trifluoroethyl)benzamide (prepared in a similar way to BH1-103) (100 mg, 0.282 mmol, 1 equiv), 4-fluoro-5-(1-methylpiperidin-4-yl)pyridin-2-amine (59.0 mg, 0.282 mmol, 1 equiv), Pd₂(dba)₃ (25.8 mg, 0.0282 mmol, 10 mol %), XPhos (26.9 mg, 0.0564 mmol, 20 mol %), and K₂CO₃ (77.9 mg, 0.564 mmol, 2 equiv). Finally, t-BuOH (3.0 mL) was added, and the suspension was degassed with argon for 15 min. The vessel was sealed, and the reaction mixture was heated to 100° C. under stirring. After 16 h, the reaction mixture was cooled down to rt and poured into water (50 mL). The suspension was filtered and the solid was washed with EtOAc and concentrated under vacuum to afford the product (78.5 mg, 0.149 mmol, 53% yield) as a yellow solid. HPLC 97% (t_{R}=13.3 min, CH₃OH 20-95% in water (0.1% TFA), 1 mL/min, 20 min). ¹H NMR (500 MHz, DMSO-d6) δ 11.96 (brs, 1H), 9.76 (s, 1H), 9.28 (t, J=6.3 Hz, 1H), 8.34 (d, J=14.0 Hz, 1H), 8.30 (d, J=8.2 Hz, 2H), 8.22 (d, J=10.9 Hz, 1H), 8.11 (d, J=8.1 Hz, 2H), 7.44 (dd, J=3.5, 2.2 Hz, 1H), 6.81 (dd, J=3.6, 1.8 Hz, 1H), 4.16 (qt, J=9.7, 4.9 Hz, 2H), 2.99-2.88 (m, 2H), 2.73-2.64 (m, 1H), 2.25 (s, 3H), 2.10-2.01 (m, 2H), 1.91-1.73 (m, 4H). ¹⁹F NMR (471 MHz, DMSO-d6) δ −70.34 (t, J=9.8 Hz), —108.58 (t, J=12.7 Hz). HPLC-MS (ESI+): m/z 527.8 (M+H)⁺. HRMS (ESI+) m/z calculated for C₂₆H₂₆F₄N₇O⁺ (M+H)⁺ 528.2129, found 528.2128.

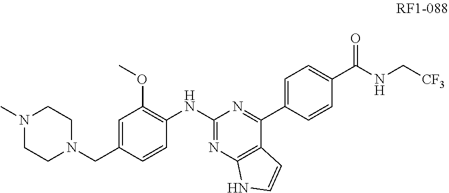 [SMILES: COc1cc(CN2CCN(C)CC2)ccc1Nc1nc(-c2ccc(C(=O)NCC(F)(F)F)cc2)c2cc[nH]c2n1]

4-(2-((2-Methoxy-4-((4-methylpiperazin-1-yl)methyl) phenyl)amino)-7H-pyrrolo[2,3-d]pyrimidin-4-yl)-N-(2,2,2-trifluoroethyl)benzamide (RF1-088): A 10-mL pressure vessel, dried and under argon atmosphere, was charged with 4-(2-chloro-7H-pyrrolo[2,3-d]pyrimidin-4-yl)-N-(2,2,2-trifluoroethyl)benzamide (prepared in a similar way to BH1-103) (100 mg, 0.282 mmol, 1 equiv), 2-methoxy-4-((4-methylpiperazin-1-yl)methyl) aniline (57.9 mg, 0.282 mmol, 1 equiv), Pd₂(dba)₃ (25.8 mg, 0.0282 mmol, 10 mol %), XPhos (26.9 mg, 0.0564 mmol, 20 mol %), and K₂CO₃ (77.9 mg, 0.564 mmol, 2 equiv). Finally, t-BuOH (3.0 mL) was added, and the suspension was degassed with argon for 15 min. The vessel was sealed, and the reaction mixture was heated to 100° C. under stirring. After 36 h, the reaction mixture was cooled down to rt, diluted with water (50 mL) and extracted with EtOAc (5×30 mL). The combined organic phases were washed with brine, dried over MgSO₄, filtered, and concentrated. The crude product was purified by column chromatography (C18, 5-95%) MeOH in water containing 0.1% TFA) to afford the product (10.5 mg, 0.0190 mmol, 7% yield) as a yellow solid. HPLC 99% (t_{R}=15.5 min, CH₃OH 20-95% in water (0.1% TFA), 1 mL/min, 20 min). ¹H NMR (500 MHz, DMSO-d6) δ 11.83 (s, 1H), 9.27 (t, J=6.3 Hz, 1H), 8.58 (d, J=8.2 Hz, 1H), 8.26 (d, J=8.1 Hz, 2H), 8.09 (d, J=8.1 Hz, 2H), 7.90 (s, 1H), 7.39 (dd, J=3.6, 2.3 Hz, 1H), 7.10 (brs, 1H), 7.01 (d, J=8.2 Hz, 1H), 6.77 (dd, J=3.7, 1.8 Hz, 1H), 4.15 (qd, J=9.7, 6.1 Hz, 2H), 3.94 (s, 3H), 3.59 (s, 2H), 3.23-2.99 (m, 8H), 2.79 (s, 3H). ¹⁹F NMR (471 MHz, DMSO-d₆) δ −70.35 (t, ³J_{F-H}=9.6 Hz). HPLC-MS (ESI+): m/z 554.3 (M+H)⁺. HRMS (ESI+) m/z calculated for C₂₈H₃₁F₃N₇O₂⁺ (M+H)⁺ 554.2486, found 554.2484.

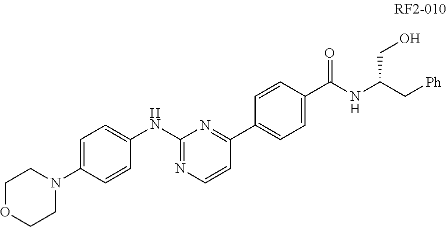 [SMILES: O=C(N[C@H](CO)Cc1ccccc1)c1ccc(-c2ccnc(Nc3ccc(N4CCOCC4)cc3)n2)cc1]

(S)—N-(1-Hydroxy-3-phenylpropan-2-yl)-4-(2-((4-morpholinophenyl)amino)pyrimidin-4-yl)-benzamide (RF2-010): BL4-102 (100 mg, 0.266 mmol) was added in to 10 mL reaction flask and dissolved in dry ACN (2 mL). To the mixture, DIPEA (46 μL, 0.27 mmol, 1 equiv), HATU (111 mg, 0.292 mmol, 1.1 equiv), and(S)-2-amino-3-phenylpropan-1-ol (44.2 mg, 0.292 mmol, 1.1 equiv) were added and stirred for 16 h at rt. The reaction mixture was diluted with EtOAc (50 mL) and washed successively with water (15 mL), Sat. ammonium chloride (15 mL), and brine (15 mL). The organic layer was dried over anhydrous Na₂SO₄ and evaporated under reduced pressure. The resulting residue was purified by flash column chromatography (SiO₂, 0-6% MeOH in DCM) to afford RF2-010 (29.2 mg, 0.0573 mmol, 22% yield) as a yellow solid. HPLC 98% (t_{R}=7.88 min, CH₃OH 5-95% in water (0.1% TFA), 1 mL/min, 20 min). ¹H NMR (500 MHz, DMSO-d₆) δ 9.48 (s, 1H), 8.53 (d, J=5.2 Hz, 1H), 8.31 (d, J=8.4 Hz, 1H), 8.21 (d, J=8.5 Hz, 2H), 7.95 (d, J=8.5 Hz, 2H), 7.68 (d, J=9.0 Hz, 2H), 7.39 (d, J=5.2 Hz, 1H), 7.37-7.31 (m, 2H), 7.31-7.23 (m, 2H), 7.16 (ddd, J=8.6, 5.8, 2.5 Hz, 1H), 6.94 (d, J=9.1 Hz, 2H), 4.86 (t, J=5.7 Hz, 1H), 4.25-4.15 (m, 1H), 3.78-3.73 (m, 4H), 3.56-3.42 (m, 2H), 3.08-3.03 (m, 4H), 3.01-2.91 (m, 1H), 2.88-2.78 (m, 1H). HPLC-MS (ESI+): m/z 510.2 (M+H)⁺. HRMS (ESI+) m/z calculated for C₃₀H₃₂N₅O₃+ (M+H)⁺ 510.2500, found 510.2494.

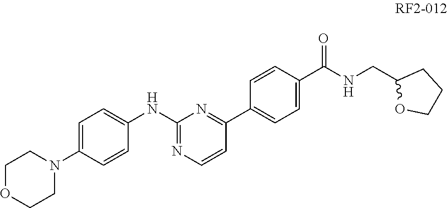 [SMILES: O=C(NCC1CCCO1)c1ccc(-c2ccnc(Nc3ccc(N4CCOCC4)cc3)n2)cc1]

4-(2-((4-Morpholinophenyl)amino)pyrimidin-4-yl)-N-((tetrahydrofuran-2-yl)methyl)-benzamide (RF2-012): BL4-102 (100 mg, 0.266 mmol) was added in to 10 mL reaction flask and dissolved in dry ACN (2 mL). To the mixture, DIPEA (46 μL, 0.27 mmol, 1 equiv), HATU (111 mg, 0.292 mmol, 1.1 equiv), and (tetrahydrofuran-2-yl) methanamine (30 μL, 0.29 mmol, 1.1 equiv) were added and stirred for 16 h at rt. The reaction mixture was diluted with EtOAc (50 mL) and washed successively with water (15 mL), Sat. ammonium chloride (15 mL), and brine (15 mL). The organic layer was dried over anhydrous Na₂SO₄ and evaporated under reduced pressure. The resulting residue was purified by flash column chromatography (SiO₂, 0-6% MeOH in DCM) to afford RF2-012 (25.0 mg, 0.0544 mmol, 20% yield) as a brown solid. HPLC 99% (t_{R}=7.97 min, CH₃OH 5-95% in water (0.1% TFA), 1 ml/min, 20 min). ¹H NMR (500 MHz, DMSO-d₆) δ 9.48 (s, 1H), 8.66 (t, J=5.8 Hz, 1H), 8.52 (d, J=5.1 Hz, 1H), 8.22 (d, J=8.5 Hz, 2H), 8.00 (d, J=8.5 Hz, 2H), 7.67 (d, J=9.0 Hz, 2H), 7.39 (d, J=5.2 Hz, 1H), 6.93 (d, J=9.1 Hz, 2H), 4.00 (p, J=6.4 Hz, 1H), 3.83-3.75 (m, 1H), 3.77-3.72 (m, 4H), 3.68-3.60 (m, 1H), 3.39-3.33 (m, 2H), 3.08-3.02 (m, 4H), 1.98-1.75 (m, 3H), 1.65-1.55 (m, 1H). ¹³C NMR (126 MHz, DMSO) δ 166.3, 163.1, 160.8, 159.7, 146.7, 139.7, 136.7, 133.4, 128.2, 127.2, 120.8, 116.1, 108.0, 77.5, 67.6, 66.7, 49.8, 38.7, 29.2, 25.6. HPLC-MS (ESI+): m/z 460.2 (M+H)⁺. HRMS (ESI+) m/z calculated for C₂₆H₃₀N₅O₃+ (M+H)⁺ 460.2343, found 510.2337.

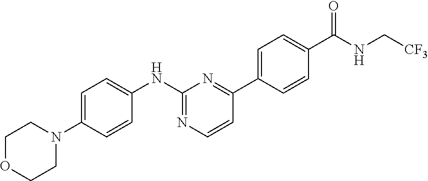 [SMILES: O=C(NCC(F)(F)F)c1ccc(-c2ccnc(Nc3ccc(N4CCOCC4)cc3)n2)cc1]

4-(2-((4-Morpholinophenyl)amino)pyrimidin-4-yl)-N-(2,2,2-trifluoroethyl)benzamide (SR8-169). The carboxylic acid BL4-102 (0.100 g, 0.265 mmol) was added to 20 mL vial and dissolved in dry DMF (2 mL). DIPEA (0.138 mL, 0.795 mmol), HATU (0.112 g, 0.292 mmol), and 2,2,2-trifluoroethylamine.HCl (0.040 g, 0.292 mmol) were then added to this mixture. The resulting mixture was stirred for 16 h at room temperature. The mixture was diluted with EtOAc (25 mL) and washed successively with water (20 mL), sat. ammonium chloride (20 mL), and brine (20 mL). The organic layer was dried (Na₂SO₄), evaporated under reduced pressure, and the resulting residue purified by flash column chromatography (SiO₂) using 0-10% gradient eluent (MeOH in DCM) to afford SR8-169 as a yellow foam (0.049 g, 40% yield). ¹H NMR (500 MHz, DMSO-d₆) δ 9.50 (s, 1H), 9.23 (t, J=6.3 Hz, 1H), 8.54 (d, J=5.1 Hz, 1H), 8.27 (d, J=8.5 Hz, 2H), 8.05 (d, J=8.5 Hz, 2H), 7.68 (d, J=9.0 Hz, 2H), 7.41 (d, J=5.2 Hz, 1H), 6.94 (d, J=9.1 Hz, 2H), 4.18-4.10 (m, 2H), 3.78-3.72 (m, 4H), 3.09-3.03 (m, 4H); ¹⁹F NMR (471 MHz, DMSO-d₆) δ −70.36 (t, J=9.6 Hz); HPLC: >99% [t_{R}=18.5 min, gradient 5-95% MeOH and water (with 0.1% TFA), 20 min]. >99% [t_{R}=10.0 min, isocratic 50% MeOH in water (with 0.1% TFA), 20 min]; HPLC-MS (ESI+): m/z 458.0 [100% (M+H)⁺], m/z 913.3 [100%, (2M−1)⁻]; HRMS (ESI+): m/z calcd for C₂₃H₂₃F₃N₅O₂ (M+H)⁺ 458.1798, found 458.1791 m/z calcd for C₂₃H₂₂F₃N₅O₂Na (M+Na)⁺ 480.1618, found 480.1611.

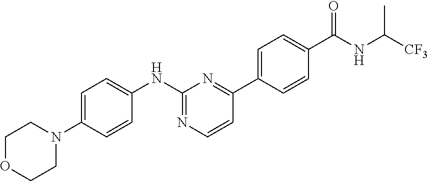 [SMILES: CC(NC(=O)c1ccc(-c2ccnc(Nc3ccc(N4CCOCC4)cc3)n2)cc1)C(F)(F)F]

(RS)-4-(2-((4-Morpholinophenyl)amino)pyrimidin-4-yl)-N-(1,1,1-trifluoropropan-2-yl)benzamide (SR8-170). The amide SR8-170 was obtained as a yellow foam (0.053 g, 42% yield) from BL4-102 (0.100 g, 0.265 mmol) and (R,S)-2-amino-1,1,1-trifluoropropane·HCl (0.044 g, 0.292 mmol) by following the procedure used to make SR8-169. ¹H NMR (500 MHz, DMSO-d₆) δ 9.51 (s, 1H), 8.99 (d, J=8.7 Hz, 1H), 8.54 (d, J=5.1 Hz, 1H), 8.27 (d, J=8.5 Hz, 2H), 8.05 (d, J=8.5 Hz, 2H), 7.68 (d, J=9.0 Hz, 2H), 7.41 (d, J=5.2 Hz, 1H), 6.94 (d, J=9.1 Hz, 2H), 4.89 (dq, J=15.8, 7.7 Hz, 1H), 3.78-3.73 (m, 4H), 3.08-3.04 (m, 4H), 1.40 (d. J=7.1 Hz, 3H); ¹⁹F NMR (471 MHz, DMSO-d₆) δ −75.55 (d, J=7.9 Hz); HPLC: >99% [t_{R}=6.3 min, isocratic 55% MeOH and 45% water (with 0.1% TFA), 20 min]; HPLC-MS (ESI+): m/z 472.0 [100% (M+H)⁺], m/z 470.2 [100%, (M−1)⁺]; HRMS (ESI+): m/z calcd for C₂₄H₂₅F₃N₅O₂ (M+H)⁺ 472.1955, found 472.1946 m/z calcd for C₂₄H₂₄F₃N₅O₂Na (M+Na)⁺ 494.1774, found 494.1765.

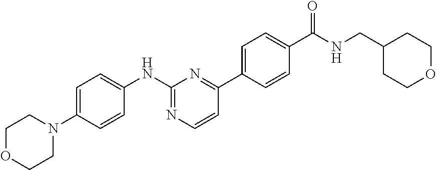 [SMILES: O=C(NCC1CCOCC1)c1ccc(-c2ccnc(Nc3ccc(N4CCOCC4)cc3)n2)cc1]

4-(2-((4-Morpholinophenyl)amino)pyrimidin-4-yl)-N-((tetrahydro-2H-pyran-4-yl)methyl)benzamide (SR8-171). The amide SR8-171 was obtained as a light brown foam (0.079 g, 63% yield) from BL4-102 (0.100 g, 0.265 mmol) and 4-aminomethyltetrahydropyran (0.034 g, 0.292 mmol) by following the procedure used to make SR8-169. ¹H NMR (500 MHz, DMSO-d₆) δ 9.49 (s, 1H), 8.62 (t, J=5.8 Hz, 1H), 8.53 (d, J=5.1 Hz, 1H), 8.23 (d, J=8.4 Hz, 2H), 8.00 (d, J=8.4 Hz, 2H), 7.68 (d, J=8.9 Hz, 2H), 7.40 (d, J=5.1 Hz, 1H), 7.03-6.87 (m, 2H), 3.96-3.83 (m, 2H), 3.80-3.64 (m, 4H), 3.20 (t, J=6.3 Hz, 2H), 3.12-2.98 (m, 4H), 1.87-1.78 (m, 1H), 1.62 (d, J=12.5 Hz, 2H), 1.31-1.14 (m, 2H); HPLC: >99% [t_{R}=4.6 min, isocratic 55% MeOH and 45% water (with 0.1% TFA), 20 min]. HPLC-MS (ESI+): m/z 474.1 [100% (M+H)⁺]; HRMS (ESI+): m/z calcd for C₂₇H₃₂N₅O₃ (M+H)⁺ 474.2500, found 474.2490 m/z calcd for C₂₇H₃₀N₅O₃ Na (M+Na)⁺ 496.2319, found 496.2315.

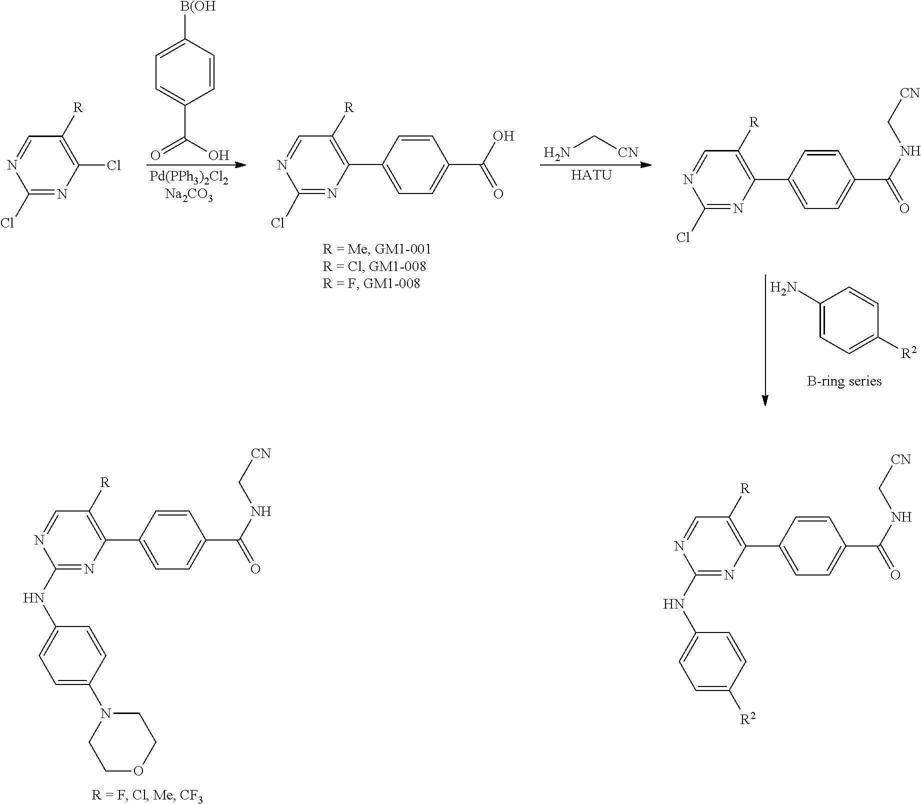

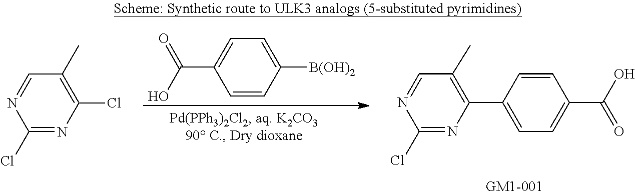

4-(2-Chloro-5-methylpyrimidin-4-yl)benzoic acid (GM1-001): A 250 ml round bottom flask was charged with potassium carbonate (13.4 g, 2 mol), deionized water (48.0 mL) and stirred until potassium carbonate was dissolved. To this mixture, added 2,4-dichloro-5-methylpyrimidine (2.0 g, 12.2 mmol) and 4-boronobenzoic acid (2.22 g, 13.4 mmol) followed by dry dioxane (72 ml) and stirred the reaction mixture vigorously. The reaction mixture was bubbled with argon for 10 min., added bis(triphenylphosphine) palladium (II) dichloride (0.430 g, 0.612 mmol) and heated at 90° C. in an oil bath. The reaction progress was in monitored by TLC (50% EtOAc/hexanes), HPLC-MS and ¹H NMR. After 2 h, the mixture was cooled to room temperature, concentrated under vacuum to remove dioxane, then acidified to pH=3 using HCl (1 M). The crude product was collected by vacuum filtration and rinsed with water (30 ml) to afford pure GM1-001 (2.93 g, 96.6%) as a white solid. ¹H NMR (500 MHz, DMSO) δ 8.77 (s, 1H), 8.09 (d, J=8.3 Hz, 2H), 7.81 (d, J=8.4 Hz, 2H), 2.35 (s, 3H). HPLC-MS (ESI+): m/z 249.1 (M+1)⁺.

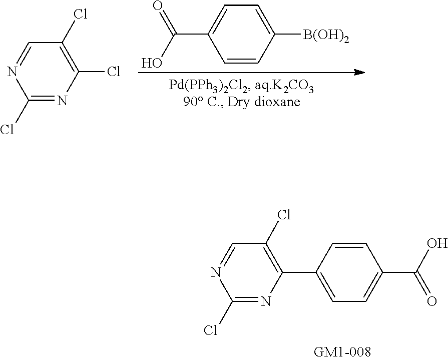

4-(2,5-Dichloropyrimidin-4-yl)benzoic acid (GM1-008): This compound was synthesized using the procedure described for GM1-001 except using potassium carbonate (12.03 g, 2 mol) in deionized water (43.6 mL), 2,4,5-trichloropyrimidine (2.0 g, 0.0109 mol), boronic acid (1.99 g, 0.0120 mol), dioxane (65 mL) and bis(triphenylphosphine) palladium (II) dichloride (0.383 g, 0.05 mol) to afford GM1-008 (2.40 g, 81.9%) as a white solid. ¹H NMR (500 MHz, DMSO) δ 9.05 (s, 1H), 8.12 (d, J=8.4 Hz, 2H), 7.94 (d, J=8.4 Hz, 2H). HPLC-MS (ESI+): m/z 269.1 (M+1)⁺, (ESI−): m/z 267.0 (M−1).

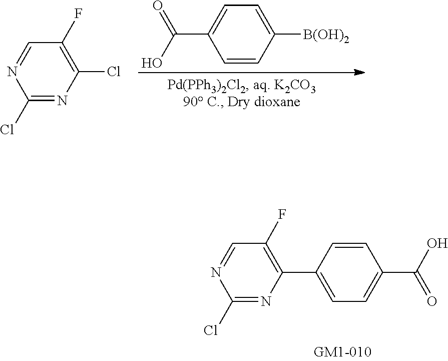

4-(2-Chloro-5-fluoropyrimidin-4-yl)benzoic acid (GM1-010): This compound was synthesized using the procedure described for GM1-001 except using potassium carbonate (13.2 g, 2.0 mol) in deionized water (47.8 mL), 2,4-dichloro-5-fluoropyrimidine (2.0 g, 0.0119 mol), boronic acid (1.99 g, 0.0120 mol), dioxane (65 mL) and bis(triphenylphosphine) palladium (II) dichloride (0.420 g, 0.00059 mmol) to afford GM1-010 (2.19 g, 72.4%) as a white solid . . . ¹H NMR (500 MHz, DMSO) § 8.99 (d, J=3.2 Hz, 1H), 8.12 (s, 4H). HPLC-MS (ESI+): m/z 253.1 (M+1)⁺, (ESI−): m/z 251.0 (M−1)⁻

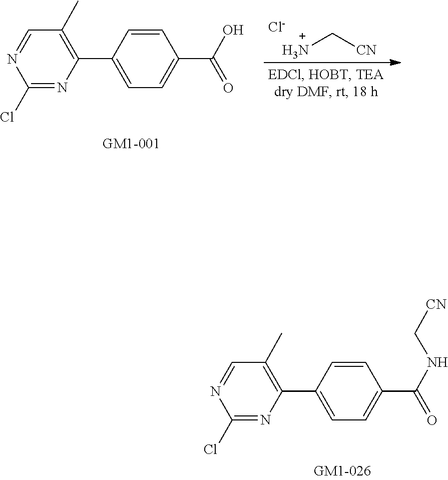

4-(2-Chloro-5-methylpyrimidin-4-yl)-N-(cyanomethyl)benzamide (GM1-026): 1-Ethyl-3(3-dimethylaminopropyl)carbodiimide (EDCl·HCl, 0.989 g, 0.0051 mol) was added to a solution of 2-aminoacetonitrile hydrochloride (0.397 g, 0.00430 mol), GM1-001 (1.0 g, 0.00344 mol) and HOBT·H₂O (0.698 g, 0.0051 mol) in dry DMF (25.4 ml) and the vessel was degassed for 10 min with argon. Triethyl amine (1.2 ml, 0.0086 mol) was added to the reaction mixture and stirred at room temperature for 12 h under argon. The reaction progress was monitored by TLC (80% EtOAc/hexanes), HPLC-MS, and ¹H NMR. Another reaction was carried out using the same equivalents of starting materials and the reaction progress was monitored as described here. The crude products from both reactions were combined, diluted with water and extracted with ethyl acetate. The organic layers were combined, washed with water and brine, dried (Na₂SO₄) and concentrated to obtain the crude product, which was purified by SiO₂ chromatography (EtOAc 0 to 30% in hexanes) to afford pure GM1-026 (1.02 g, 44.3%) as a white solid. ¹H NMR (500 MHz, DMSO) δ 9.38 (t, J=5.5 Hz, 1H), 8.77 (s, 1H), 8.02 (d, J=8.5 Hz, 1H), 7.83 (d, J=8.5 Hz, 2H), 4.36 (d, J=5.4 Hz, 2H), 2.36 (s, 3H). HPLC-MS (ESI+): m/z 287.2 (M+1)⁺

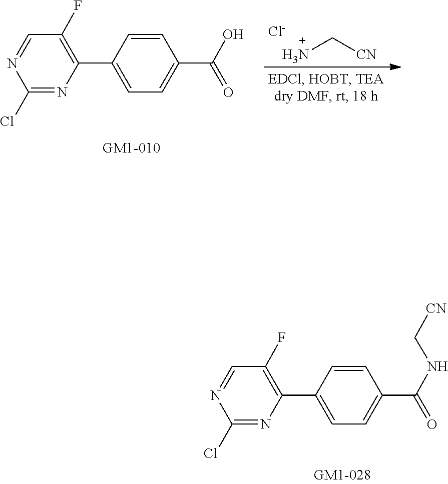

4-(2-Chloro-5-fluoropyrimidin-4-yl)-N-(cyanomethyl)benzamide (GM1-028): This compound was synthesized using the procedure described for GM1-026 except using EDC·HCl (2.28 g, 0.0119 mol), 2-aminoacetonitrile hydrochloride (0.917 g, 0.0099 mol), GM1-010 (2.00 g, 0.00793 mol) and HOBT (1.60 g, 0.0119 mol) in dry DMF (58.7 ml) and triethylamine (2.78 ml, 0.0198 mol) to obtain pure GM1-028 (1.10 g, 47.8%) as a white solid. ¹H NMR (500 MHz, DMSO) δ 9.43 (s, 1H), 9.03 (d, J=3.0 Hz, 2H), 8.19-8.13 (m, 2H), 8.10-8.05 (m, 2H), 4.37 (d, J=5.3 Hz, 2H). ¹⁹F NMR (471 MHz, CDCl₃) δ −138.59 (d, J=3.1 Hz). HPLC-MS (ESI+): m/z 291.2 (M+1)⁺, 289.1 (M−1)⁻.

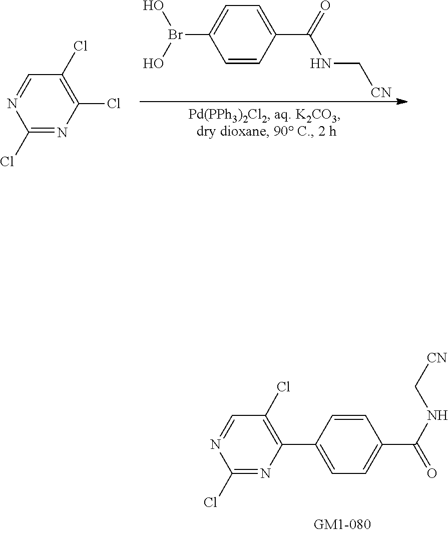

N-(Cyanomethyl)-4-(2,5-dichloropyrimidin-4-yl)benzamide (GM1-080): A 250 ml round bottom flask was charged with potassium carbonate (3.01 g, 2 mol), deionized water (10.9 ml) and stirred until potassium carbonate was dissolved. To this mixture, added 2,4,5-trichloropyrimidine (0.500 g, 2.73 mmol), (4-((cyanomethyl)carbamoyl)phenyl)boronic acid (0.557 g, 2.73 mmol) followed by dry dioxane (27.3 ml). The reaction mixture was bubbled with argon for 10 min, added bis(triphenylphosphine) palladium (II) dichloride (0.959 g, 0.136 mmol). The reaction mixture was heated at 90° C. in an oil bath and the reaction progress was monitored with TLC (50% EtOAc/hexanes), HPLC-MS and ¹H NMR. After 2 h, the flask was cooled to room temperature, mixture was concentrated to remove dioxane, and diluted with ethyl acetate (30 mL). The organic layer was separated, dried over sodium sulfate, and concentrated under reduced pressure, and the residue obtained was purified by SiO₂ column chromatography to afford GM1-080 as a white solid (0.504 g, 60.1%). ¹H NMR (500 MHz, DMSO) δ 9.41 (t, J=5.5 Hz, 1H), 9.06 (s, 1H), 8.07-8.03 (m, 2H), 7.97-7.94 (m, 2H), 4.37 (d, J=5.5 Hz, 2H). HPLC-MS (ESI+): m/z 307.0 (M+1)⁺, 305.1 (M−1)⁻.

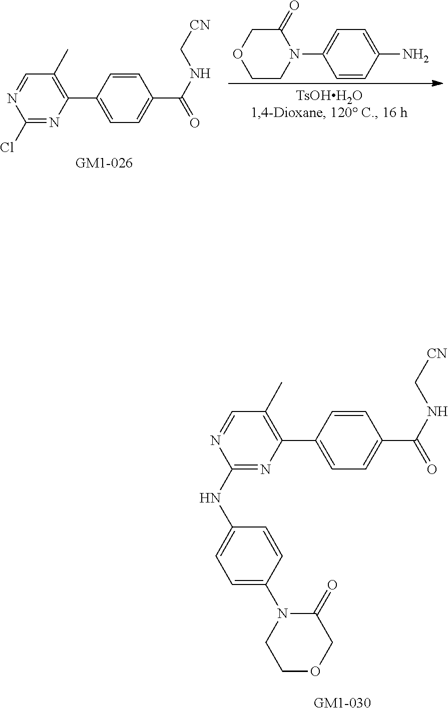 [SMILES: Cc1cnc(Cl)nc1-c1ccc(C(=O)NCC#N)cc1 ; Nc1ccc(N2CCOCC2=O)cc1 ; Cc1cnc(Nc2ccc(N3CCOCC3=O)cc2)nc1-c1ccc(C(=O)NCC#N)cc1]

N-(Cyanomethyl)-4-(5-methyl-2-((4-(3-oxomorpholino) phenyl)amino)pyrimidin-4-yl)benzamide (GM1-030): 2-Chloro-N-(cyanomethyl)-5-methylpyrimidine-4-carboxamide GM1-026 (0.080 g, 0.279 mmol), 4-(4-aminophenyl) morpholin-3-one (0.0398 g, 0.206 mmol) in 1,4-dioxane (1.84 mL) were stirred in a round bottom flask. Para-toluenesulfonic acid monohydrate (0.0318 g. 0.167 mmol) was added to the suspension under stirring and the reaction refluxed for 16 h under argon atmosphere. The reaction progress was monitored by TLC (100% EtOAc), HPLC-MS and ¹H NMR. After 16 h the flask was cooled to room temperature and concentrated. The mixture was diluted with ethyl acetate (20 mL) and washed with 5% aqueous sodium bicarbonate solution (20 mL). The organic layer was separated, dried over sodium sulfate, and concentrated under reduced pressure, and the residue obtained was purified using SiO₂ chromatography to afford GM1-030 as a pale yellow solid (0.030 g, 26.5%). ¹H NMR (500 MHz, DMSO) δ 9.72 (s, 1H), 9.34 (t, J=5.5 Hz, 1H), 8.47 (d, J=0.8 Hz, 1H), 8.01 (d, J=8.4 Hz, 2H), 7.84-7.73 (m, 4H), 7.30-7.21 (m, 2H), 4.36 (d, J=5.5 Hz, 2H), 4.18 (s, 2H), 3.99-3.91 (m, 2H), 3.76-3.63 (m, 2H), 2.23 (s, 3H). HPLC-MS (ESI+): m/z 443.2 (M+1)⁺. HPLC purity: 100%. HRMS (ESI+): m/z calcd for C₂₄H₂₂N₆O₃ (M+H)⁺ 443.1826, found 443.1821.

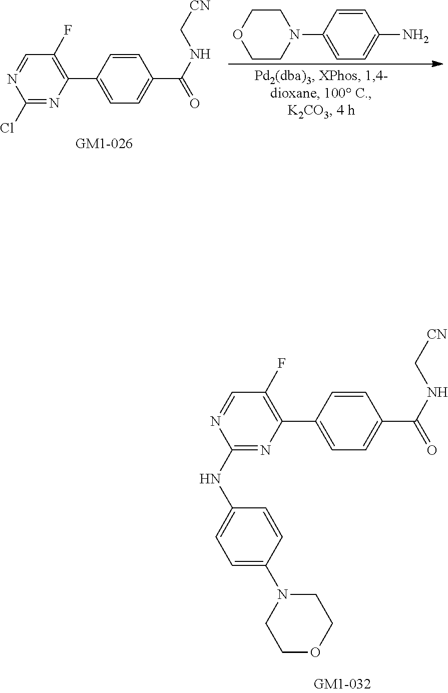 [SMILES: N#CCNC(=O)c1ccc(-c2nc(Cl)ncc2F)cc1 ; Nc1ccc(N2CCOCC2)cc1 ; N#CCNC(=O)c1ccc(-c2nc(Nc3ccc(N4CCOCC4)cc3)ncc2F)cc1]

N-(Cyanomethyl)-4-(5-methyl-2-((4-(morpholino) phenyl)amino)pyrimidin-4-yl)benzamide (GM1-032): The GM1-026 (0.075 g, 0.2615 mmol), 4-morpholinoaniline (0.0465 g, 0.2615 mmol), X-Phos (0.0249 g, 0.0523 mmol), K₂CO₃ (0.0721 g, 0.523 mmol) and dry 1,4-dioxane (1.99 mL, 0.131 M) were added to a 10 mL microwave vial under argon and the mixture was degassed by bubbling argon for 10 min. The Pd₂(dba)₃ (0.0239 g, 0.0523 mmol) was added under argon, vial was sealed and stirred at 100° C. The reaction progress was monitored by TLC (80% EtOAc/hexanes), HPLC-MS and ¹H NMR. After 4 h, the mixture was cooled to room temperature, diluted with ethyl acetate (10 mL) and filtered through celite. The residue was partitioned between ethyl acetate and water and extracted with EtOAc (3×10 ml), and combined organic layers were dried over Na₂SO₄. The ethyl acetate layer was concentrated under reduced pressure, and the residue obtained was purified using SiO₂ column chromatography to afford GM1-032 as a yellow solid (46 mg, 35.6%). ¹H NMR (500 MHz, CDCl₃) δ 9.36 (s, 1H), 9.33 (t, J=5.5 Hz, 1H), 8.39 (s, 1H), 8.00 (d, J=8.5 Hz, 2H), 7.79 (d, J=8.4 Hz, 2H), 7.62 (d, J=9.1 Hz, 1H), 6.88 (d, J=9.1 Hz, 2H), 4.36 (d, J=5.5 Hz, 2H), 3.73 (dd, J=5.8, 3.8 Hz, 4H), 3.05-2.98 (m, 4H), 2.20 (s, 3H). HPLC-MS (ESI+): m/z 429.2 (M+1)⁺. HPLC purity: 99.795%. HRMS (ESI+): m/z calcd for C₂₄H₂₄N₆O₂ (M+H)⁺ 429.2033, found 429.2028.

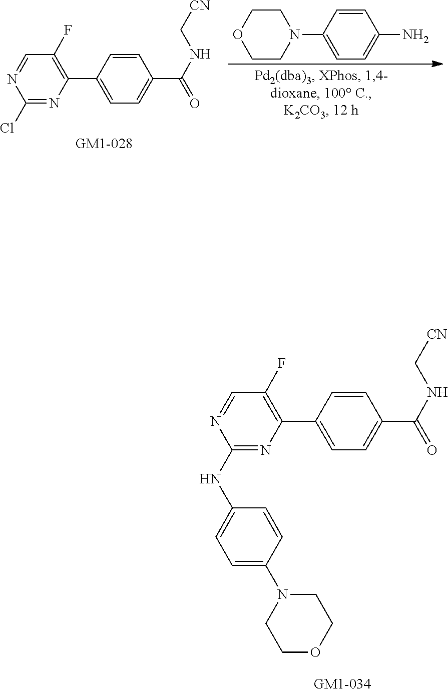 [SMILES: N#CCNC(=O)c1ccc(-c2nc(Cl)ncc2F)cc1 ; Nc1ccc(N2CCOCC2)cc1 ; N#CCNC(=O)c1ccc(-c2nc(Nc3ccc(N4CCOCC4)cc3)ncc2F)cc1]

N-(Cyanomethyl)-4-(5-fluoro-2-((4-morpholinophenyl)amino)pyrimidin-4-yl)benzamide (GM1-034): The GM1-028 (0.075 g, 0.258 mmol), 4-morpholinoaniline (0.0459 g, 0.258 mmol), X-Phos (0.0246 g, 0.0516 mmol), potassium carbonate (0.0713 g, 0.517 mmol) and dry 1,4-dioxane (1.96 mL, 0.131 M) were added to a 10 mL microwave vial under argon and the mixture was degassed by bubbling argon for 10 min. The Pd₂(dba)₃ (0.0236 g, 0.0258 mmol) was added under argon, vial was sealed and stirred at 100° C. The reaction progress was monitored by TLC (80% EtOAc/hexanes), HPLC-MS and ¹H NMR. After 12 h, the mixture was cooled to room temperature, diluted with ethyl acetate (10 mL) and filter through celite. The residue was partitioned between ethyl acetate and water and extracted with EtOAc (3×10 ml), and combined organic layers were dried over Na₂SO₄. The ethyl acetate layer was evaporated, and the residue obtained was purified by SiO₂ chromatography to afford GM1-034 as a yellow solid (38 mg, 34.2%). ¹H NMR (500 MHz, DMSO) & 9.59 (s, 1H), 9.38 (t, J=5.5 Hz, 1H), 8.62 (d, J=3.3 Hz, 1H), 8.16-8.12 (m, 2H), 8.05 (d, J=8.6 Hz, 2H), 7.61 (d, J=9.0 Hz, 2H), 6.93 (d, J=9.1 Hz, 2H), 4.37 (d, J=5.4 Hz, 2H), 3.77-3.73 (m, 4H), 3.06-3.03 (m, 4H). ¹⁹F NMR (500 MHz, DMSO) δ −151.97. HPLC-MS (ESI+): m/z 433.2 (M+1)⁺. HPLC purity: 99.9%. HRMS (ESI+): m/z calcd for C₂₃H₂₁FN₆O₂ (M+H)⁺ 433.1782, found 433.1777.

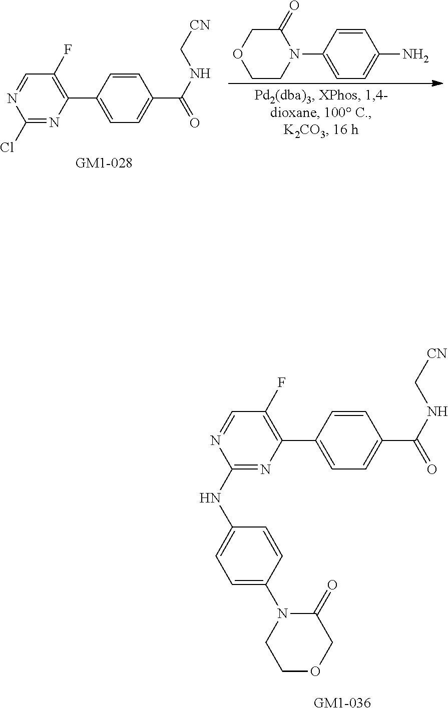 [SMILES: N#CCNC(=O)c1ccc(-c2nc(Cl)ncc2F)cc1 ; Nc1ccc(N2CCOCC2=O)cc1 ; N#CCNC(=O)c1ccc(-c2nc(Nc3ccc(N4CCOCC4=O)cc3)ncc2F)cc1]

N-(Cyanomethyl)-4-(5-fluoro-2-((4-(3-oxomorpholino) phenyl)amino)pyrimidin-4-yl)benzamide (GM1-036): 2-Chloro-N-(cyanomethyl)-5-fluoropyrimidine-4-carboxamide GM1-028 (0.070 g, 0.241 mmol), 4-(4-aminophenyl) morpholin-3-one (0.0463 g, 0.241 mmol), X-Phos (0.0230 g, 0.048 mmol), potassium carbonate (0.0666 g, 0.482 mmol) and dry 1,4-dioxane (1.83 mL, 0.131 M) were added to a 10 mL microwave vial under argon and the mixture was degassed by bubbling argon for 10 min. The Pd₂(dba)₃ (0.0221 g, 0.0241 mmol) was added under argon and the vial was sealed and stirred at 100° C. Reaction progress was monitored by TLC (10% MeOH/DCM), HPLC-MS and ¹H NMR. After 16 h, the mixture was cooled to room temperature and dioxane was removed under reduced pressure. The product was triturated with 30% methanol and dichloromethane (3×20 mL). Supernatant was decanted and the residue was dried under vacuum to afford GM1-036BA2 as a yellow solid (35 mg, 32.7%). ¹H NMR (500 MHz, DMSO) δ 9.94 (s, 1H), 9.41 (s, 1H), 8.71 (d, J=3.3 Hz, 1H), 8.16 (d, J=8.0 Hz, 2H), 8.09-8.05 (m, 2H), 7.79 (d, J=8.9 Hz, 2H), 7.32 (d, J=8.9 Hz, 2H), 4.37 (s, 2H), 4.20 (s, 2H), 3.98 (dd, J=6.0, 4.1 Hz, 2H), 3.72 (dd, J=6.0, 4.1 Hz, 2H). ¹⁹F NMR (471 MHz, DMSO) δ −150.50. HPLC-MS (ESI+): m/z 447.2 (M+1)⁺. HPLC purity: 99.99%. HRMS (ESI+): m/z calcd for C₂₃H₁₉FN₆O₃ (M+H)⁺ 447.1575, found 447.1578.

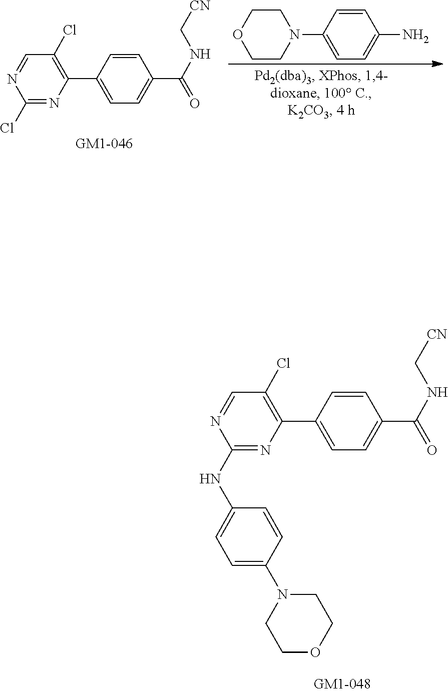 [SMILES: N#CCNC(=O)c1ccc(-c2nc(Cl)ncc2Cl)cc1 ; Nc1ccc(N2CCOCC2)cc1 ; N#CCNC(=O)c1ccc(-c2nc(Nc3ccc(N4CCOCC4)cc3)ncc2Cl)cc1]

4-(5-Chloro-2-((4-morpholinophenyl)amino)pyrimidin-4-yl)-N-(cyanomethyl)benzamide (GM1-048): N-(cyanomethyl)-4-(2,5-dichloropyrimidin-4-yl)benzamide, GM1-046, (0.050 g. 0.162 mmol), 4-morpholinoaniline (0.02609 g, 0.146 mmol), X-Phos (0.0155 g, 0.0325 mmol), potassium carbonate (0.0449 g, 0.325 mmol) and dry 1,4-dioxane (1.23 mL, 0.131 M) were added to a 10 mL microwave vial under argon and the mixture was degassed by bubbling argon for 10 min. The Pd₂(dba)₃ (0.0149 g, 0.0162 mmol) was added under argon and the vial was sealed and stirred at 100° C. The reaction progress was monitored by TLC (80% EtOAc/hexanes), HPLC-MS and ¹H NMR. After 16 h, the mixture was cooled to room temperature, diluted with ethyl acetate (10 mL) and filtered through celite. The residue was partitioned between ethyl acetate and water. The product was extracted with EtOAc (3×10 ml) and combined organic layers were dried over Na₂SO₄. The ethyl acetate layer was concentrated under reduced pressure, and the residue obtained was purified by SiO₂ column chromatography to afford GM1-048 as a yellow solid (24 mg, 32.8%). ¹H NMR (500 MHz, DMSO) δ 9.76 (s, 1H), 9.36 (t, J=5.5 Hz, 1H), 8.59 (s, 1H), 8.02 (d, J=8.0 Hz, 2H), 7.91 (d, J=8.0 Hz, 2H), 7.58 (d, J=8.6 Hz, 2H), 6.91 (d, J=8.5 Hz, 2H), 4.37 (d, J=5.4 Hz, 2H), 3.79-3.64 (m, 4H), 3.04 (m, 4H). HPLC-MS (ESI+): m/z 449.2 (M+1)⁺. HPLC purity: 100%. HRMS (ESI+): m/z calcd for C₂₃H₂₁ClN₆O₂ (M+H)⁺ 449.1487, found 449.1576.

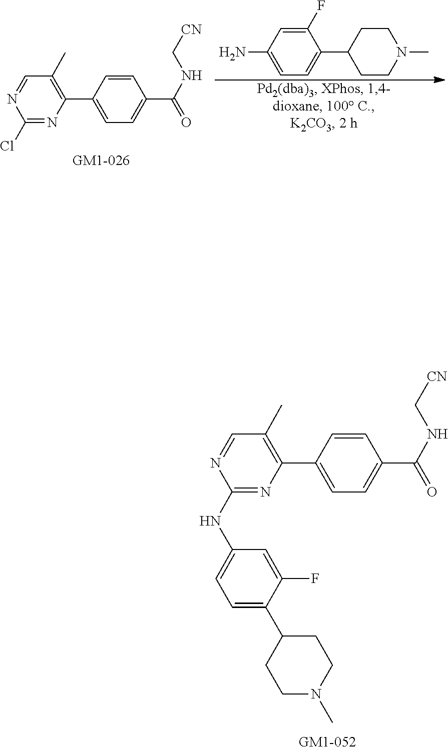 [SMILES: Cc1cnc(Cl)nc1-c1ccc(C(=O)NCC#N)cc1 ; CN1CCC(c2ccc(N)cc2F)CC1 ; Cc1cnc(Nc2ccc(C3CCN(C)CC3)c(F)c2)nc1-c1ccc(C(=O)NCC#N)cc1]

N-(Cyanomethyl)-4-(2-((3-fluoro-4-(1-methylpiperidin-4-yl)phenyl)amino)-5-methylpyrimidin-yl)benzamide (GM1-052B2): 4-(2-Chloro-5-methylpyrimidin-4-yl)-N-(cyanomethyl)benzamide, GM1-026 (0.070 g, 0.244 mmol), 3-fluoro-4-(1-methylpiperidin-4-yl) aniline (0.0457 g, 0.219 mmol), X-Phos (0.0233 g, 0.0489 mmol), potassium carbonate (0.0675 g, 0.489 mmol) and dry 1,4-dioxane (1.86 mL) were added to a 10 mL microwave vial under argon and the mixture was degassed by bubbling argon for 10 min. The Pd₂(dba)₃ (0.0224 g, 0.0244 mmol) was added under argon, vial was sealed and stirred at 100° C. The reaction progress was monitored by TLC (10% MeOH/DCM), HPLC-MS and ¹H NMR. After 2 h, the mixture was cooled to room temperature, diluted with ethyl acetate (10 mL) and filtered through celite and rinsed the celite pad with ethyl acetate. The residue was partitioned between ethyl acetate and water and the product was extracted with EtOAc (3×10 ml) and combined organics were dried over Na₂SO₄. The ethyl acetate layer was removed under reduced pressure, and the residue obtained was purified by SiO₂ chromatography to afford GM1-052B2 as a yellow solid (34 mg, 22.2%). ¹H NMR (500 MHz, DMSO) δ 9.78 (s, 1H), 9.36 (t, J=5.5 Hz, 1H), 8.48 (s, 1H), 8.02 (d, J=7.8 Hz, 2H), 7.81 (d, J=7.9 Hz, 2H), 7.75 (d, J=13.9 Hz, 1H), 7.47-7.43 (m, 1H), 7.19 (t, J=8.7 Hz, 1H), 4.37 (d, J=5.5 Hz, 2H), 2.88 (d, J=10.9 Hz, 2H), 2.66 (dt, J=10.7, 5.3 Hz, 1H), 2.22 (d, J=11.3 Hz, 6H), 2.04-1.94 (m, 2H), 1.75-1.62 (m, 4H). ¹⁹F NMR (500 MHz, DMSO) δ −118.85. HPLC-MS (ESI+): m/z 459.3 (M+1)⁺. HPLC purity: 98.67%. HRMS (ESI+): m/z calcd for C₂₆H₂₇FN₆O (M+H)⁺ 459.2303, found 459.2297.

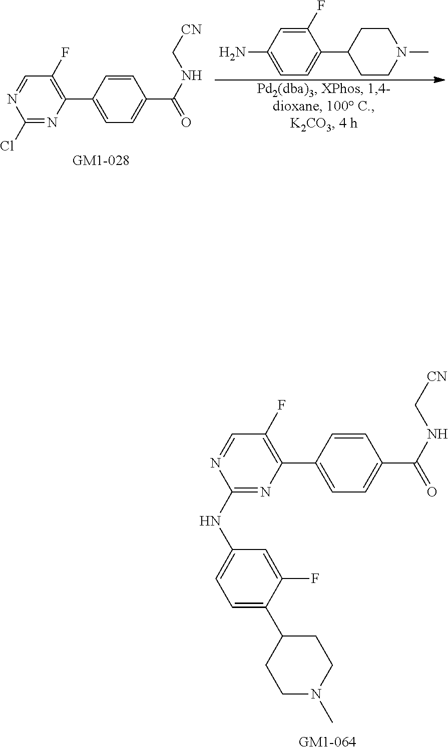 [SMILES: N#CCNC(=O)c1ccc(-c2nc(Cl)ncc2F)cc1 ; CN1CCC(c2ccc(N)cc2F)CC1 ; CN1CCC(c2ccc(Nc3ncc(F)c(-c4ccc(C(=O)NCC#N)cc4)n3)cc2F)CC1]

N-(Cyanomethyl)-4-(5-fluoro-2-((3-fluoro-4-(1-methylpiperidin-4-yl)phenyl) amino)pyrimidin-yl)benzamide (GM1-064): 4-(2-Chloro-5-fluoropyrimidin-4-yl)-N-(cyanomethyl)benzamide, GM1-028, (0.070 g, 0.240 mmol), 3-fluoro-4-(1-methylpiperidin-4-yl) aniline MA9-058-6, (0.0451 g, 0.216 mmol), X-Phos (0.0230 g, 0.0480 mmol), potassium carbonate (0.0664 g, 0.481 mmol) and dry 1,4-dioxane (1.89 mL, 0.131 M) were added to a 10 mL microwave vial under argon and the mixture was degassed by bubbling argon for 10 min. The Pd₂(dba)₃ (0.0220 g, 0.0240 mmol) was added under argon and the vial was sealed and stirred at 100° C. The reaction progress was monitored by TLC (20% MeOH/DCM), HPLC-MS and ¹H NMR. After 4 h, the mixture was cooled to room temperature, diluted with ethyl acetate (10 mL) and filtered through celite (the celite was rinsed with EtOAc). The residue was partitioned between ethyl acetate and water. The product was extracted with EtOAc (3×10 ml) and combined organics were dried over Na₂SO₄. The organic layer was evaporated under reduced pressure, and the residue obtained was purified by SiO₂ chromatography to afford GM1-064 as a yellow solid (28 mg, 25.4%). ¹H NMR (500 MHz, DMSO) δ 10.00 (s, 1H), 9.40 (t, J=5.5 Hz, 1H), 8.72 (d, J=3.2 Hz, 1H), 8.15 (d, J=8.0 Hz, 2H), 8.07 (d, J=8.5 Hz, 2H), 7.72 (dd, J=13.7, 2.2 Hz, 1H), 7.46 (dd, J=8.5, 2.2 Hz, 1H), 7.24 (t, J=8.7 Hz, 1H), 4.37 (d, J=5.4 Hz, 2H), 2.88 (dd, J=9.1, 5.7 Hz, 2H), 2.71-2.61 (m, 1H), 2.20 (s, 3H), 1.97 (dt, J=11.0, 5.2 Hz, 2H), 1.70 (tt, J=8.6, 3.8 Hz, 4H). ¹⁹F NMR (500 MHz, DMSO) δ −118.76, −150.21. HPLC-MS (ESI+): m/z 463.2 (M+1)⁺. HPLC purity: 99.19%. HRMS (ESI+): m/z calcd for C₂₅H₂₄F₂N₆O (M+H)⁺ 463.2052, found 463.2034.

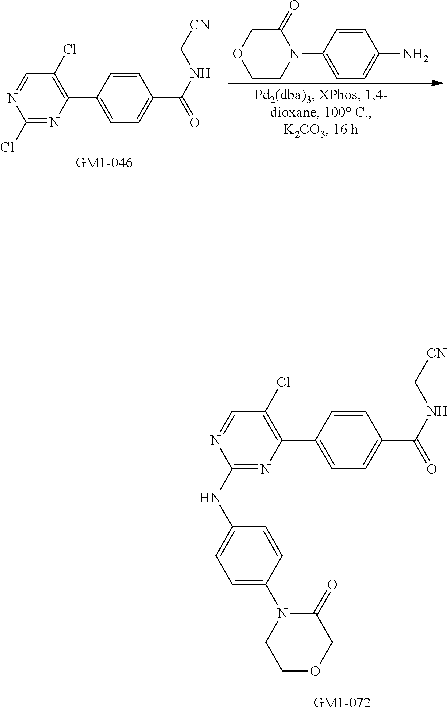 [SMILES: N#CCNC(=O)c1ccc(-c2nc(Cl)ncc2Cl)cc1 ; Nc1ccc(N2CCOCC2=O)cc1 ; N#CCNC(=O)c1ccc(-c2nc(Nc3ccc(N4CCOCC4=O)cc3)ncc2Cl)cc1]

4-(5-Chloro-2-((4-(3-oxomorpholino) phenyl)amino)pyrimidin-4-yl)-N-(cyanomethyl)benzamide (GM1-072)N-(cyanomethyl)-4-(2,5-dichloropyrimidin-4-yl)benzamide GM1-046, (0.050 g, 0.162 mmol), 4-(4-aminophenyl) morpholin-3-one (0.0281 g, 0.146 mmol), X-Phos (0.0155 g, 0.0325 mmol), potassium carbonate (0.0449 g, 0.325 mmol) and dry 1,4-dioxane (1.23 mL, 0.131 M) were added to a 10 mL microwave vial under argon and the mixture was degassed by bubbling argon for 10 min. The Pd₂(dba)₃ (0.0149 g, 0.0162 mmol) was added under argon and then vial was sealed and stirred at 100° C. The reaction progress was monitored by TLC (100% EtOAc/hexanes), HPLC-MS and ¹H NMR. After 16 h, the mixture was cooled to room temperature, diluted with ethyl acetate (10 mL) and filter through celite (the celite pad was rinsed with EtOAc). The residue was partitioned between ethyl acetate and water. The product extracted with EtOAc (3×10 ml) and combined organics were dried over Na₂SO₄, concentrated under reduced pressure, and the residue obtained was purified by SiO₂ chromatography to afford GM1-072 as a yellow solid (18 mg, 24.3%). ¹H NMR (500 MHz, DMSO) δ 10.09 (s, 1H), 9.37 (t, J=5.5 Hz, 1H), 8.68 (s, 1H), 8.09-8.00 (m, 3H), 7.96-7.91 (m, 2H), 7.76 (d, J=8.9 Hz, 1H), 7.31 (d, J=8.9 Hz, 2H), 4.37 (d, J=5.4 Hz, 2H), 4.19 (s, 2H), 3.97 (dd, J=6.0, 4.2 Hz, 2H), 3.70 (dd, J=6.0, 4.2 Hz, 2H). HPLC-MS (ESI+): m/z 463.2 (M+1)⁺. HPLC purity: 99.74%. HRMS (ESI+): m/z calcd for C₂₃H₁₉ClN₆O₃ (M+H)⁺ 463.1279, found 463.1283.

### Biological Data

| | | IC₅₀ for | | | IC₅₀ for | |
|---|---|---|---|---|---|---|
| | | ULK3 | | | JAK2 | |
| | | Reaction | DSF | DSF | Reaction | |
| | | Biology | (ULK3) | (JAK2) | Biology | GI₅₀ |
| Name | | kinase | ΔTm | ΔTm | kinase | (MM |
| Molecular Wt (Amt. Supplied mg) | | assay | (° C.) | (° C.) | assay | cell) |
| RF1-046 | M. Wt. = 496.49 | 7.26 nM | 16.7 | | 0.745 nM | |
| | | | 13.3 | | | |
| 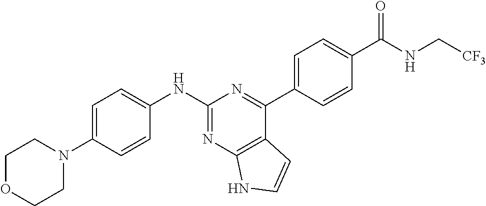 [SMILES: O=C(NCC(F)(F)F)c1ccc(-c2nc(Nc3ccc(N4CCOCC4)cc3)nc3[nH]ccc23)cc1] | | | | | | |
| RF1-052 | M. Wt. = 523.56 | 5.43 nM | 16.7 | | | |
| | | | 13.2 | | | |
| 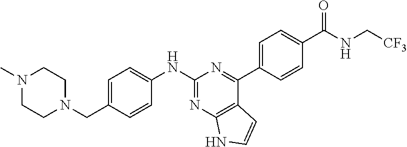 [SMILES: CN1CCN(Cc2ccc(Nc3nc(-c4ccc(C(=O)NCC(F)(F)F)cc4)c4cc[nH]c4n3)cc2)CC1] | | | | | | |
| BL4-139 | M. Wt = 453.51 | 10.4 nM | 15.0 | | <0.51 nM | |
| | | ULK1 | | | | |
| 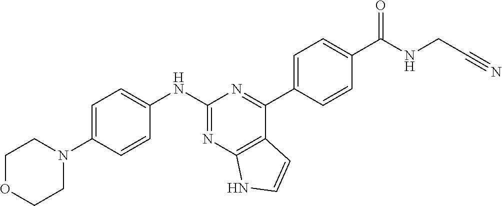 [SMILES: N#CCNC(=O)c1ccc(-c2nc(Nc3ccc(N4CCOCC4)cc3)nc3[nH]ccc23)cc1] | | 1400 ULK2 1180 | | | | |
| SR8-169 | M. Wt = 457.46 | 0.91 nM | 15.0 | | <0.508 nM | U266: |
| | | 10.3 nM | 11.7 | | | 86.18 |
| 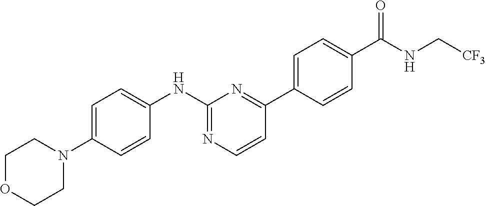 [SMILES: O=C(NCC(F)(F)F)c1ccc(-c2ccnc(Nc3ccc(N4CCOCC4)cc3)n2)cc1] | | | 14.7 | | | nM 8226: 83.55 nM OPM2: 15.11 nM U266 PSR: 4.406 μM 8226 B25: |
| | | | | | | 213.9 |
| | | | | | | nM |
| RF1-024 | M. Wt. = 480.58 | 18.4 nM | 15.0 | | <0.508 nM | U266: |
| | | | 12.6 | | | 917 |
| 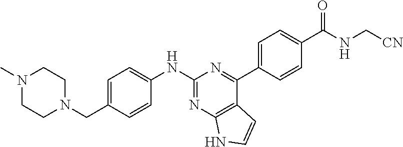 [SMILES: CN1CCN(Cc2ccc(Nc3nc(-c4ccc(C(=O)NCC#N)cc4)c4cc[nH]c4n3)cc2)CC1] | | | 16.5 | | | nM 8226: 3.634 μM OPM2: 159 nM U266 PSR: 2.583 |
| | | | | | | μM |
| | | | | | | 8226 |
| | | | | | | B25: |
| | | | | | | 9.494 |
| | | | | | | μM |
| RF1-034 | M. Wt. = 483.55 | 9.48 nM | 14.6 | | | |
| | | | 13.6 | | | |
| 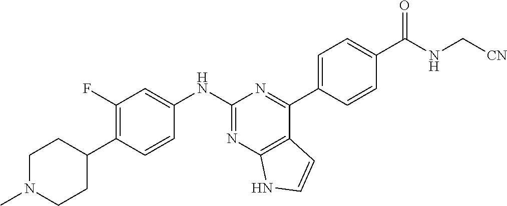 [SMILES: CN1CCC(c2ccc(Nc3nc(-c4ccc(C(=O)NCC#N)cc4)c4cc[nH]c4n3)cc2F)CC1] | | | | | | |
| Momelotinib (BL4-106) | M. Wt = 414.47 | 82.38 nM | 14.3 | | 2.76 nM | |
| | | | 10.6 | | | |
| 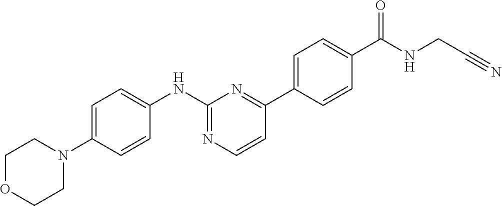 [SMILES: N#CCNC(=O)c1ccc(-c2ccnc(Nc3ccc(N4CCOCC4)cc3)n2)cc1] | | | 13.2 | | | |
| BL4-106 HCl salt | M. Wt = 487.39 | | 14.3 | | | |
| | | | 9.9 | | | |
| 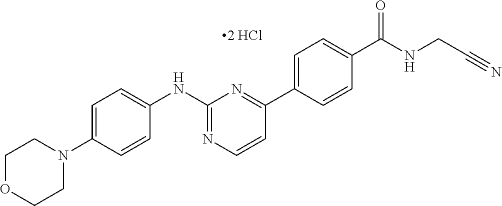 [SMILES: N#CCNC(=O)c1ccc(-c2ccnc(Nc3ccc(N4CCOCC4)cc3)n2)cc1] | | | | | | |
| SR8-170 | M. Wt = 471.48 | 0.87 nM | 14.3 | | 1.31 nM | U266 |
| | | 8.71 nM | 14.4 | | | PSR: |
| 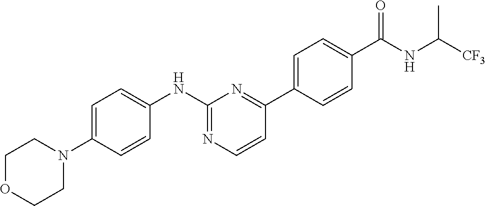 [SMILES: CC(NC(=O)c1ccc(-c2ccnc(Nc3ccc(N4CCOCC4)cc3)n2)cc1)C(F)(F)F] | | | | | | 1.579 μM 8226 B25: 404.3 nM U266: 210.0 nM 8226: 289.6 nM OPM2: |
| | | | | | | 6.698 |
| | | | | | | μM |
| MA9-060 | M. Wt = 657.21 | 35.6 nM | 11.9 | | | |
| | | | 12.6 | | | |
| 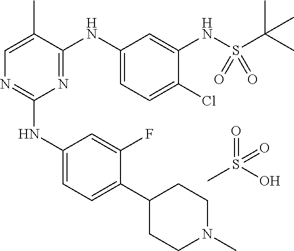 | | | | | | |
| SR8-171 | M. Wt = 473.58 | 55 nM | 11.1 | | | |
| 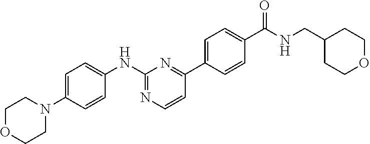 [SMILES: O=C(NCC1CCOCC1)c1ccc(-c2ccnc(Nc3ccc(N4CCOCC4)cc3)n2)cc1] | | | | | | |
| SG3-179 | M. Wt = 604.14 | 291 nM | 10.6 | | | |
| 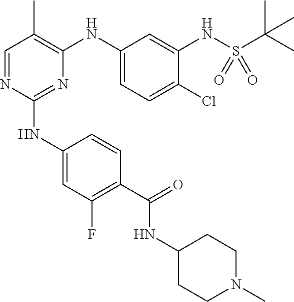 [SMILES: Cc1cnc(Nc2ccc(C(=O)NC3CCN(C)CC3)c(F)c2)nc1Nc1ccc(Cl)c(NS(=O)(=O)C(C)(C)C)c1] | | | | | | |
| RF1-058 | M. Wt. = 484.54 | | 6.8 | | | |
| 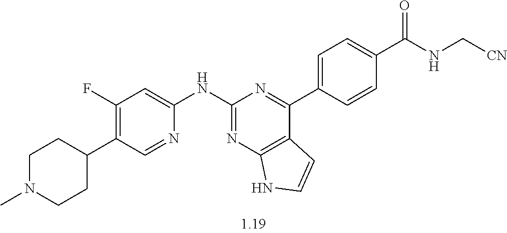 [SMILES: CN1CCC(c2cnc(Nc3nc(-c4ccc(C(=O)NCC#N)cc4)c4cc[nH]c4n3)cc2F)CC1] | | | | | | |
| RF1-060 | M. Wt. = 527.53 | 5770 nM | 6.8 | | | U266 |
| | | | | | | PSR: |
| 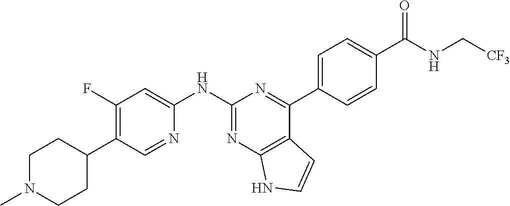 [SMILES: CN1CCC(c2cnc(Nc3nc(-c4ccc(C(=O)NCC(F)(F)F)cc4)c4cc[nH]c4n3)cc2F)CC1] | | | | | | 870.4 nM 8226 B25: 2.933 μM U266: 4.601 μM 8226: 12.00 μM OPM2: |
| | | | | | | 909.4 |
| | | | | | | nM |
| BL4-142 | M. Wt = 454.53 | 299 nM | | | | |
| 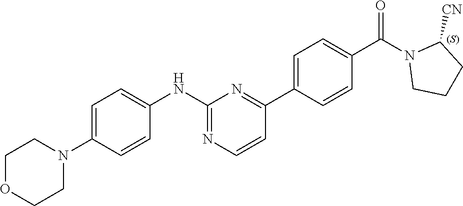 | | | | | | |
| BL4-143 | M. Wt = 454.53 | 50.4 nM | | | | |
| 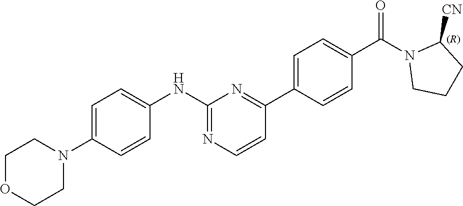 | | | | | | |
| BL4-110 | M. Wt = 440.51 | 177 nM | 9.0 | | | |
| 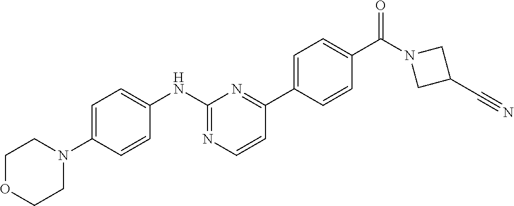 [SMILES: N#CC1CN(C(=O)c2ccc(-c3ccnc(Nc4ccc(N5CCOCC5)cc4)n3)cc2)C1] | | | | | | |
| BL4-136 | M. Wt = 277.29 | 588 nM | 5.1 | 1 | | |
| 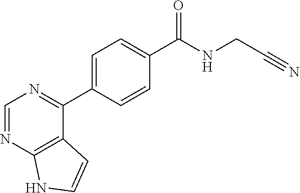 [SMILES: N#CCNC(=O)c1ccc(-c2ncnc3[nH]ccc23)cc1] | | | | | | |
| SG3-014 MSA salt | M. Wt = 640.22 | 51.9 nM | | | | |
| 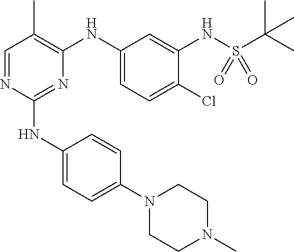 [SMILES: Cc1cnc(Nc2ccc(N3CCN(C)CC3)cc2)nc1Nc1ccc(Cl)c(NS(=O)(=O)C(C)(C)C)c1] | | | | | | |
| 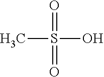 | | | | | | |
| BL4-153 | M. Wt = 490.57 | 6.5 nM | 11.6 | | | |
| 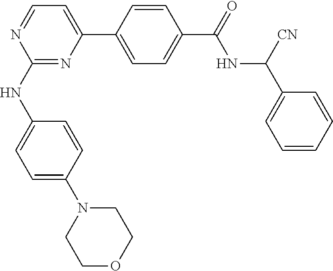 [SMILES: N#CC(NC(=O)c1ccc(-c2ccnc(Nc3ccc(N4CCOCC4)cc3)n2)cc1)c1ccccc1] | | | | | | |
| BL4-157 | M. Wt = 495.58 | 117 nM | 8.1 | | | |
| 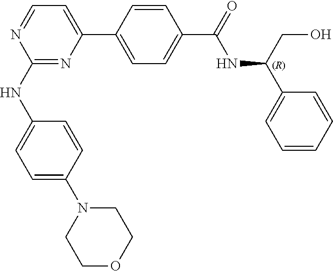 | | | | | | |
| BL4-158 | M. Wt = 495.58 | 3.7 nM | 10.7 | | | |
| 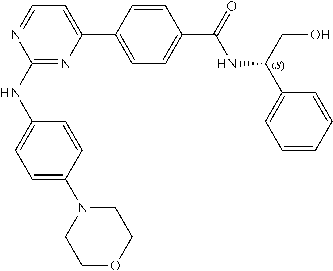 | | | | | | |
| BR1-008 | M. Wt = 509.61 | 2.28 μM | | 0.5 | | |
| 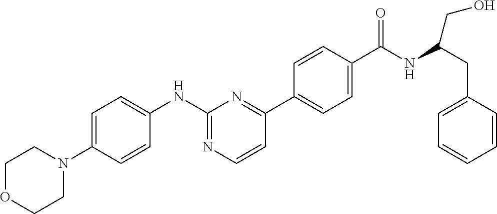 | | | | | | |
| BR1-012 | M. Wt = 523.64 | Not | 5.8 | 0.1 | | |
| | | Detemined | | | | |
| 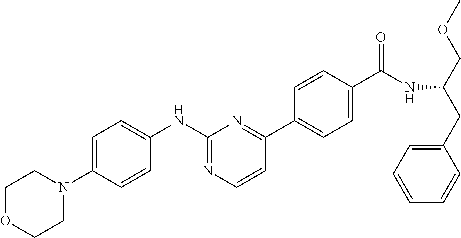 [SMILES: COC[C@H](Cc1ccccc1)NC(=O)c1ccc(-c2ccnc(Nc3ccc(N4CCOCC4)cc3)n2)cc1] | | | | | | |
| BR1-022B2 | M. Wt. = 457.46 | 5.32 nM | | 1.9 | | |
| 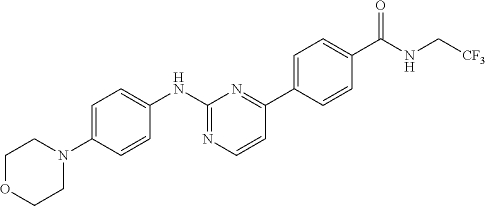 [SMILES: O=C(NCC(F)(F)F)c1ccc(-c2ccnc(Nc3ccc(N4CCOCC4)cc3)n2)cc1] | | | | | | |
| BH1-062 | MW = 475.59 | 17.1 nM | 10.7 | 1.0 | 12.9 nM | |
| 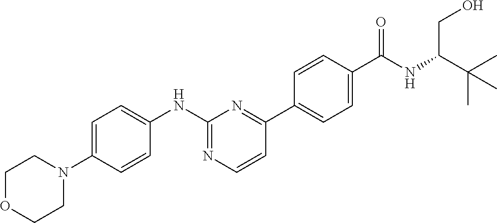 [SMILES: CC(C)(C)[C@@H](CO)NC(=O)c1ccc(-c2ccnc(Nc3ccc(N4CCOCC4)cc3)n2)cc1] | | | | | | |
| BH1-063 | MW = 461.57 | | 7.9 | 3.6 | | |
| 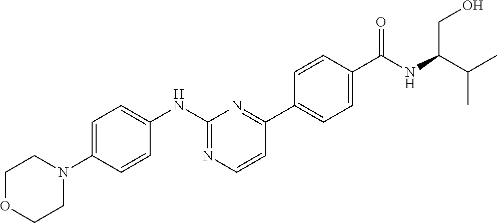 [SMILES: CC(C)[C@H](CO)NC(=O)c1ccc(-c2ccnc(Nc3ccc(N4CCOCC4)cc3)n2)cc1] | | | | | | |
| BH1-090 | MW = 428.45 | | 7.5 | 1.3 | | |
| 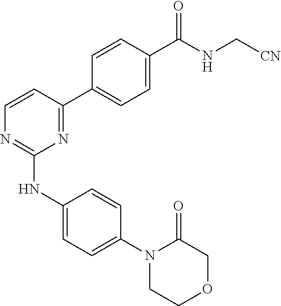 [SMILES: N#CCNC(=O)c1ccc(-c2ccnc(Nc3ccc(N4CCOCC4=O)cc3)n2)cc1] | | | | | | |
| GM1-030 | MW = 442.48 | | 8.1 | 5.5 | | |
| 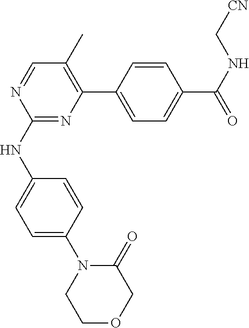 [SMILES: Cc1cnc(Nc2ccc(N3CCOCC3=O)cc2)nc1-c1ccc(C(=O)NCC#N)cc1] | | | | | | |
| GM1-032 | MW = 428.50 | | 8.4 | 7.0 | | |
| 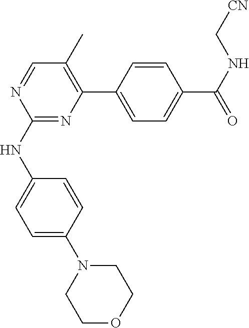 [SMILES: Cc1cnc(Nc2ccc(N3CCOCC3)cc2)nc1-c1ccc(C(=O)NCC#N)cc1] | | | | | | |
| GM1-034 | MW = 432.46 | | 10.6 | 1.4 | | |
| 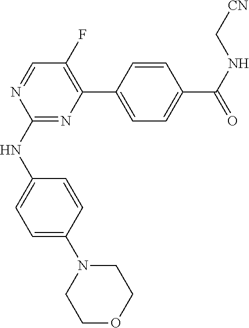 [SMILES: N#CCNC(=O)c1ccc(-c2nc(Nc3ccc(N4CCOCC4)cc3)ncc2F)cc1] | | | | | | |
| GM1-036BA2 | MW = 446.44 | | 8.7 | 1.1 | | |
| 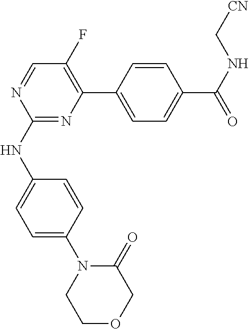 [SMILES: N#CCNC(=O)c1ccc(-c2nc(Nc3ccc(N4CCOCC4=O)cc3)ncc2F)cc1] | | | | | | |
| BH1-095 | MW = 463.49 | | 8.8 | | | |
| 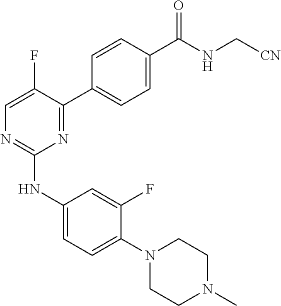 [SMILES: CN1CCN(c2ccc(Nc3ncc(F)c(-c4ccc(C(=O)NCC#N)cc4)n3)cc2F)CC1] | | | | | | |
| BH1-102 | MW = 455.57 | | 7.2 | 4.68 | | |
| 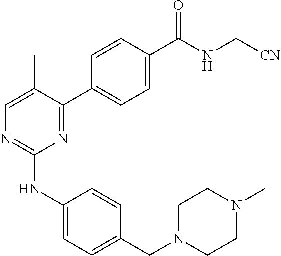 [SMILES: Cc1cnc(Nc2ccc(CN3CCN(C)CC3)cc2)nc1-c1ccc(C(=O)NCC#N)cc1] | | | | | | |
| BH1-104 | MW = 448.38 | | 6.9 | 0.05 | | |
| 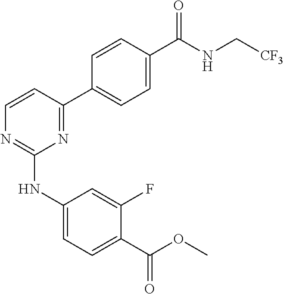 [SMILES: COC(=O)c1ccc(Nc2nccc(-c3ccc(C(=O)NCC(F)(F)F)cc3)n2)cc1F] | | | | | | |
| GM1-048 | MW = 448.91 | | 11.0 | 6.48 | | |
| | | | 13.1 | | | |
| 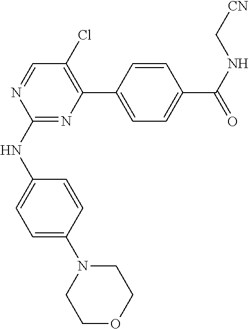 [SMILES: N#CCNC(=O)c1ccc(-c2nc(Nc3ccc(N4CCOCC4)cc3)ncc2Cl)cc1] | | | | | | |
| GM1-052B2 | MW = 458.54 | | 6.9 | 5.46 | | |
| 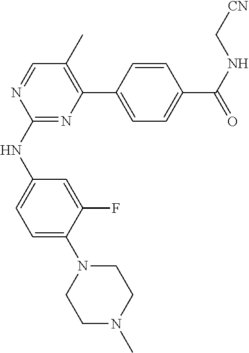 [SMILES: Cc1cnc(Nc2ccc(N3CCN(C)CC3)c(F)c2)nc1-c1ccc(C(=O)NCC#N)cc1] | | | | | | |
| GM1-064 | MW = 462.50 | | 8.0 | 3.68 | | |
| 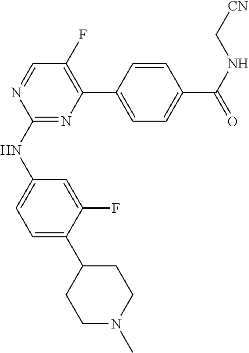 [SMILES: CN1CCC(c2ccc(Nc3ncc(F)c(-c4ccc(C(=O)NCC#N)cc4)n3)cc2F)CC1] | | | | | | |
| BH1-106 | MW = 447.39 | | 9.9 | 0.54 | | |
| 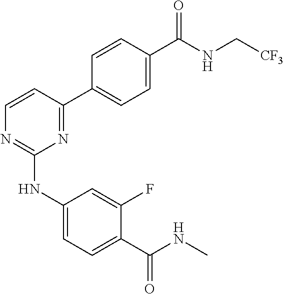 [SMILES: CNC(=O)c1ccc(Nc2nccc(-c3ccc(C(=O)NCC(F)(F)F)cc3)n2)cc1F] | | | | | | |
| GM1-072 | MW = 462.89 | | 11.8 | | | |
| 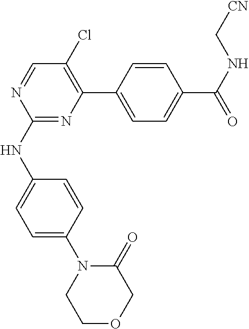 [SMILES: N#CCNC(=O)c1ccc(-c2nc(Nc3ccc(N4CCOCC4=O)cc3)ncc2Cl)cc1] | | | | | | |
| BH1-132 | M. W. = 487.48 | | 10.0 | 0.1 | | |
| 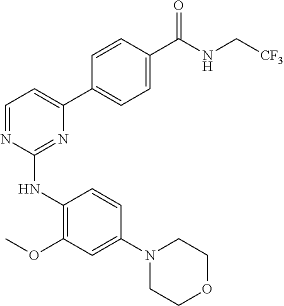 [SMILES: COc1cc(N2CCOCC2)ccc1Nc1nccc(-c2ccc(C(=O)NCC(F)(F)F)cc2)n1] | | | | | | |
| BH1-138 | M. W. = 475.45 | | | 0.5 | | |
| 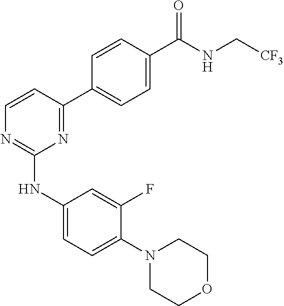 [SMILES: O=C(NCC(F)(F)F)c1ccc(-c2ccnc(Nc3ccc(N4CCOCC4)c(F)c3)n2)cc1] | | | | | | |
| BH1-150 | M. W. = 531.07 | 84.1 nM | 15.0 | 2.1 | 1.25 nM | |
| 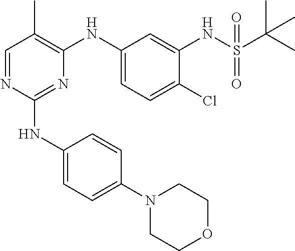 [SMILES: Cc1cnc(Nc2ccc(N3CCOCC3)cc2)nc1Nc1ccc(Cl)c(NS(=O)(=O)C(C)(C)C)c1] | | | | | | |
| BH1-151 | M. W. = 471.48 | 0.693 nM | 14.0 | 0.4 | 1.13 nM | |
| 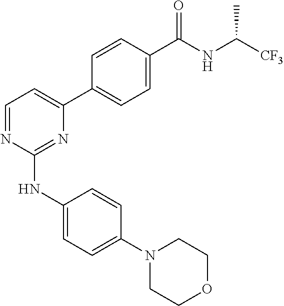 [SMILES: C[C@@H](NC(=O)c1ccc(-c2ccnc(Nc3ccc(N4CCOCC4)cc3)n2)cc1)C(F)(F)F] | | | | | | |
| BH1-152 | M. W. = 471.48 | 51.4 nM | 12.5 | 0.7 | 0.746 nM | |
| 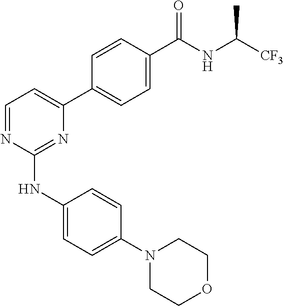 [SMILES: C[C@H](NC(=O)c1ccc(-c2ccnc(Nc3ccc(N4CCOCC4)cc3)n2)cc1)C(F)(F)F] | | | | | | |
| BH1-159 | M. W. = 497.52 | 1070 nM | 11.8 | 1.1 | 74.2 nM | |
| 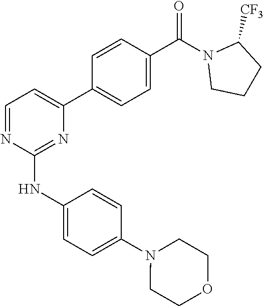 [SMILES: O=C(c1ccc(-c2ccnc(Nc3ccc(N4CCOCC4)cc3)n2)cc1)N1CCC[C@H]1C(F)(F)F] | | | | | | |
| BH1-160 | M. W. = 497.52 | 3.98 nM | 15.5 | 1.4 | 8.56 nM | |
| | | | 14.7 | | | |
| 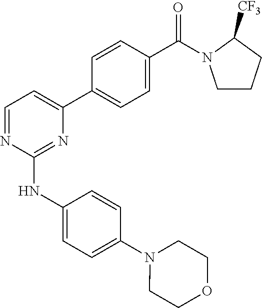 [SMILES: O=C(c1ccc(-c2ccnc(Nc3ccc(N4CCOCC4)cc3)n2)cc1)N1CCC[C@@H]1C(F)(F)F] | | | | | | |
| BR1-034 | M. Wt. = 541.67 | | 9.1 | | | |
| 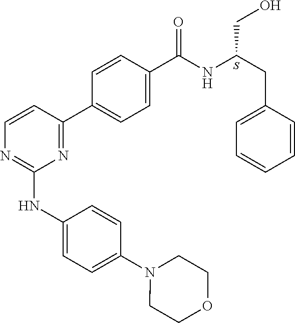 | | | | | | |
| BR1-036 | M. Wt. = 541.67 | | 9.8 | | | |
| 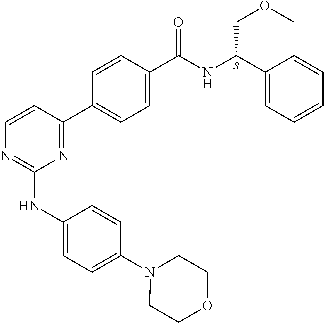 | | | | | | |
| BR1-038 | M. Wt. = 541.67 | | 8.4 | | | |
| 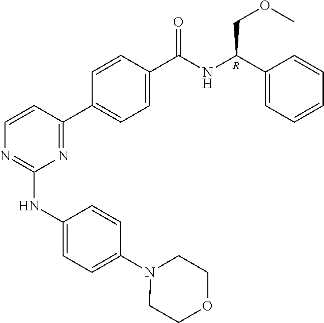 | | | | | | |
| BH2-010 | M. W. = 470.50 | | 14.4 | | | |
| 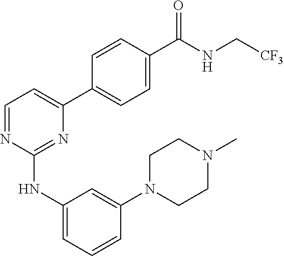 [SMILES: CN1CCN(c2cccc(Nc3nccc(-c4ccc(C(=O)NCC(F)(F)F)cc4)n3)c2)CC1] | | | | | | |
| BR1-045 | M. W. = 457.46 | | 14.7 | | | |
| 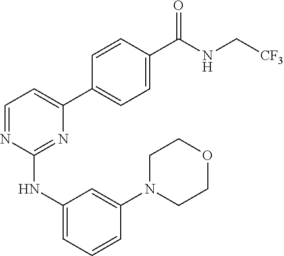 [SMILES: O=C(NCC(F)(F)F)c1ccc(-c2ccnc(Nc3cccc(N4CCOCC4)c3)n2)cc1] | | | | | | |
| BH2-011 | M. W. = 510.56 | | 14.5 | | | |
| | | | 14.4 | | | |
| 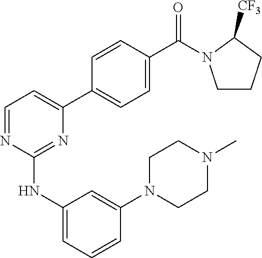 [SMILES: CN1CCN(c2cccc(Nc3nccc(-c4ccc(C(=O)N5CCC[C@@H]5C(F)(F)F)cc4)n3)c2)CC1] | | | | | | |
| BR1-047 | M. Wt. = 497.52 | | 13.4 | | | |
| 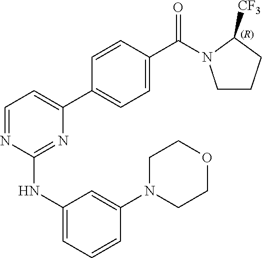 | | | | | | |
| BR1-048 | M. Wt. = 497.52 | | 9.3 | | | |
| 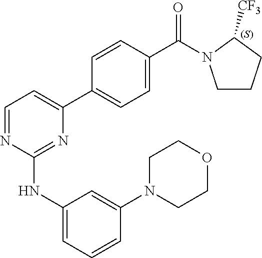 | | | | | | |
| BR1-051 | M. Wt. = 488.49 | | 11.1 | | | |
| 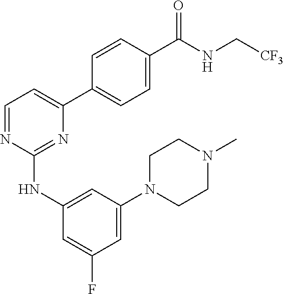 [SMILES: CN1CCN(c2cc(F)cc(Nc3nccc(-c4ccc(C(=O)NCC(F)(F)F)cc4)n3)c2)CC1] | | | | | | |
| BR1-052 | M. Wt. = 528.56 | | 14.1 | | | |
| 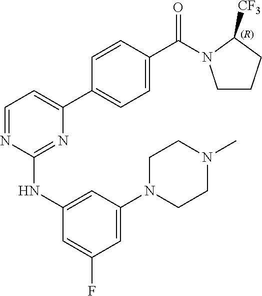 | | | | | | |
| BR1-053 | M. Wt. = 528.56 | | 10.5 | | | |
| 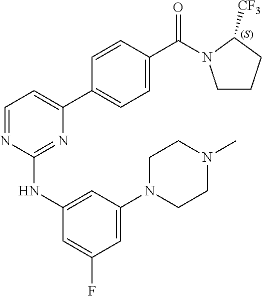 | | | | | | |
| BR1-054 | M. Wt. = 542.56 | | 1.3 | | | |
| 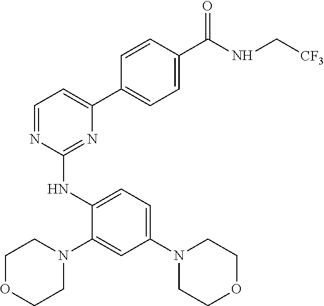 [SMILES: O=C(NCC(F)(F)F)c1ccc(-c2ccnc(Nc3ccc(N4CCOCC4)cc3N3CCOCC3)n2)cc1] | | | | | | |
| BH2-013 | M. Wt. = 510.56 | | 10.9 | | | |
| 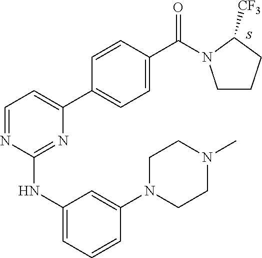 | | | | | | |
| BR1-055 | M. Wt. = 475.45 | | | | | |

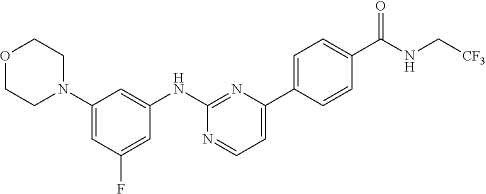 [SMILES: O=C(NCC(F)(F)F)c1ccc(-c2ccnc(Nc3cc(F)cc(N4CCOCC4)c3)n2)cc1]

IC₅₀: ULK3, ULK2, ULK1 and JAK2 (³²P Hotspot assay, Reaction Biology)
Control Staurosporine 0.14 nM (JAK2 carried @ 1 μM ATP)(8-2-22, RB); 10.3 nM (ULK1 @ 10 μM ATP)(8-2-22, RB); 1.80 nM (ULK2 @ 10 μM ATP)(8-2-22, RB); 2.36 nM (ULK3 @ 1 μM ATP)(8-2-22, RB); 6.09 nM (ULK3 @ 10 μM ATP)(8-2-22)
MA9-060, SG3-179 and Momelotinib (BL4-106) are control compounds for assessing ULK3 activity
ULK3 K_{d} determined by Microscale thermophoresis (MST)

### REFERENCES FROM EXAMPLES

The following are incorporated herein in their entirety for all purposes:
1. Sun T, Xu J, Ji M, Wang P. A Novel and Efficient Synthesis of Momelotinib. Journal of Chemical Research. 2016; 40 (8): 511-3. doi: 10.3184/174751916X14682453281177.
2. Huang Y, Dong G, Li H, Liu N, Zhang W, Sheng C. Discovery of Janus Kinase 2 (JAK2) and Histone Deacetylase (HDAC) Dual Inhibitors as a Novel Strategy for the Combinational Treatment of Leukemia and Invasive Fungal Infections. J Med Chem. 2018; 61 (14): 6056-74. Epub 20180714. doi: 10.1021/acs.jmedchem.8b00393. PubMed PMID: 29940115.
3. Wan Z, Vazquez M L, Li X, inventors; Lynk Pharmaceuticals Co., Ltd., assignee. Benzamides of pyrazolyl-amino-pyrimidinyl derivatives, and compositions and methods thereof patent WO2020119819. 2020.
4. Lawandi J, Toumieux S, Seyer V, Campbell P, Thielges S, Juillerat-Jeanneret L, Moitessier N. Constrained peptidomimetics reveal detailed geometric requirements of covalent prolyl oligopeptidase inhibitors. J Med Chem. 2009; 52 (21): 6672-84. doi: 10.1021/jm901013a. PubMed PMID: 19888757.
5. Burns C J, Palmer J T, McNally M, inventors; Cytopia Research Pty. Ltd., assignee. Preparation of 2-anilino-4-phenylpyrimidines as retrometabolic drugs for treatment of immunological or inflammatory disease, pulmonary arterial hypertension, asthma, chronic obstructive pulmonary disease, and cancer patent WO2009029998. 2009.
6. Burns C J, Bourke D G, Andrau L, Bu X, Charman S A, Donohue A C, Fantino E, Farrugia M, Feutrill J T, Joffe M, Kling M R, Kurek M, Nero T L, Nguyen T, Palmer J T, Phillips I, Shackleford D M, Sikanyika H, Styles M, Su S, Treutlein H, Zeng J, Wilks A F, Phenylaminopyrimidines as inhibitors of Janus kinases (JAKs). Bioorg Med Chem Lett. 2009; 19 (20): 5887-92. Epub 20090823. doi: 10.1016/j.bmcl.2009.08.071. PubMed PMID: 19762238.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only some representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound of Formula II or Formula III 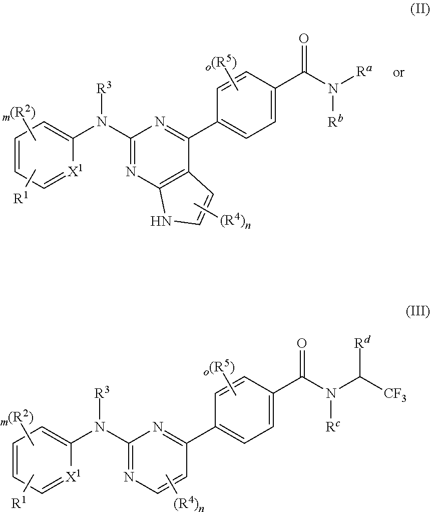
or
or a pharmaceutically acceptable salt or derivative thereof,
wherein:
m is 0, 1, 2, or 3;
n is 0, 1, or 2;
o is 0, 1, 2, 3, or 4;
R¹ is selected from —(C₀-C₃alkyl)(3- to 8-membered monocyclic or bicyclic heterocycle), R^{x}O—C(O)—(C₀-C₃ alkyl)-, and (R^{x}R^{y}N)—C(O)—(C₀-C₃ alkyl)-, each of which may be optionally substituted with one or more groups selected from Z as allowed by valency;
X¹ is selected from N or C(R²);
R², R⁴, and R⁵ are each independently selected at each occurrence from hydrogen, halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂-(C₀-C₃ alkyl)-, (R^{x}R^{y}N) S(O)₂-(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂-O—(C₀-C₃ alkyl)-, R^{z}S(O)₂-(R^{x}N)—(C₀-C₃ alkyl)-, R^{z}C(O)—(C₀-C₆ alkyl)-, R^{z}S(O)—(C₀-C₃ alkyl)-, and R^{z}S(O)₂-(C₀-C₃ alkyl)-, each of which may be optionally substituted with one or more groups selected from Z as allowed by valency;
R³ is hydrogen or C₁-C₃ alkyl;
R^{a} and R^{b} are independently selected from hydrogen, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, and (5- to 10-membered monocyclic or bicyclic heteroaryl)-(C₀-C₃ alkyl)-, each of which may be optionally substituted with one or more groups selected from Z as allowed by valency;
or R^{a} and R^{b} are brought together with the nitrogen to which they are attached to form 3- to 8-membered monocyclic or bicyclic heterocycle optionally substituted with one or more groups selected from Z as allowed by valency;
R^{c} and R^{d} are independently selected from hydrogen and C₁-C₃ alkyl; or
R^{c} and R^{d} are brought together with the atoms to which they are attached to form a 3- to 8-membered monocyclic or bicyclic heterocycle optionally substituted with one or more groups selected from Z as allowable by valency;
Z is independently selected at each occurrence from hydrogen, halo, cyano, azido, oxo, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂-(C₀-C₃ alkyl)-, (R^{x}R^{y}N) S(O)₂-(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂-O—(C₀-C₃ alkyl)-, R^{z}S(O)₂-(R^{x}N)—(C₀-C₃ alkyl)-, R^{z}C(O)—(C₀-C₆ alkyl)-, R^{z}S(O)—(C₀-C₃ alkyl)-, and R^{z}S(O)₂-(C₀-C₃ alkyl)-, each of which is optionally substituted with one or more groups selected from Y as allowed by valency;
R^{x} and R^{y} are independently selected at each occurrence from hydrogen, C₁-C₆alkyl, C₁-C₆ haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

2. The compound of claim 1, or a pharmaceutically acceptable salt or derivative thereof, wherein

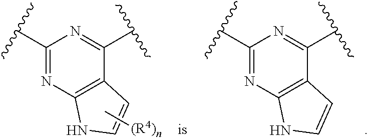

3. The compound of claim 1, or a pharmaceutically acceptable salt or derivative thereof, wherein is selected from:

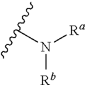

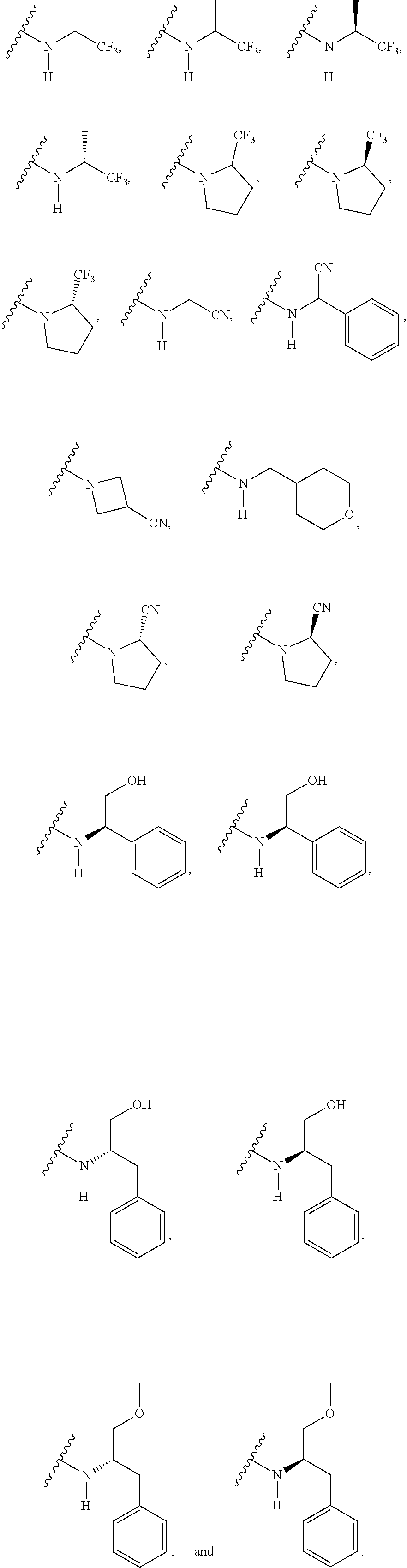

4. The compound of claim 1, or a pharmaceutically acceptable salt or derivative thereof, wherein 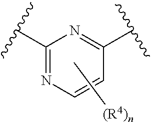
is selected from:

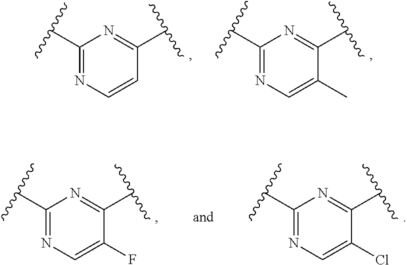

5. The compound of claim 1, or a pharmaceutically acceptable salt or derivative thereof, wherein 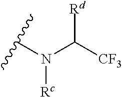
is selected from:

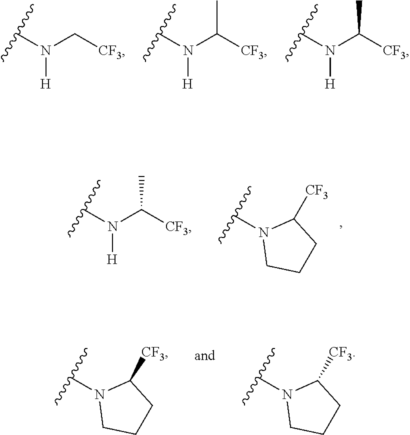

6. The compound of claim 1, or a pharmaceutically acceptable salt or derivative thereof, wherein R¹ is 5- to 6-membered monocyclic heterocycle, —(CH₂)(5- to 6-membered monocyclic heterocycle), R^{x}O—C(O)—(C₀-C₃ alkyl)-, or (R^{x}R^{y}N)—C(O)—(C₀-C₃ alkyl.

7-9. (canceled)

10. The compound of claim 1, or a pharmaceutically acceptable salt or derivative thereof, wherein R¹ is selected from:

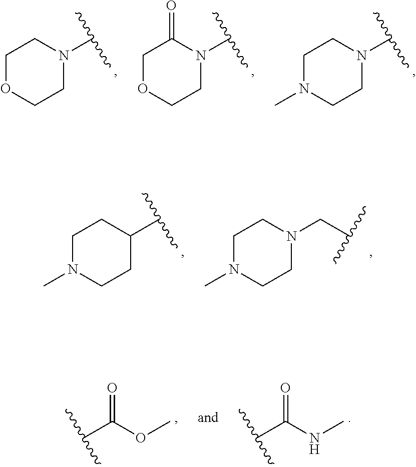

11. The compound of claim 1, or a pharmaceutically acceptable salt or derivative thereof, wherein 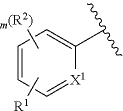
is selected from:

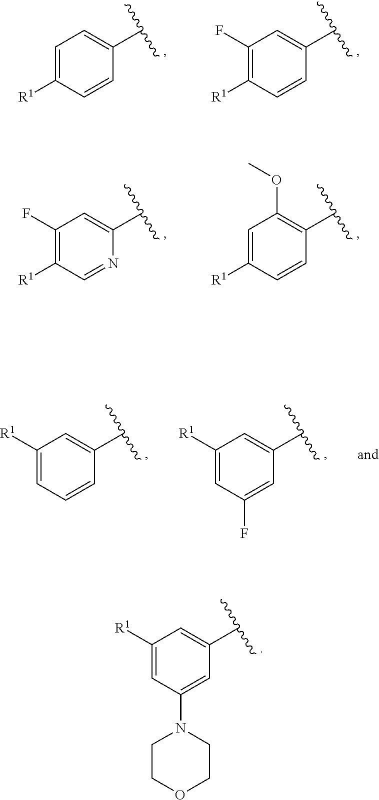

12. The compound of claim 1, or a pharmaceutically acceptable salt or derivative thereof, wherein R³ is hydrogen or methyl.

13-14. (canceled)

15. The compound of any one of claim 1, or a pharmaceutically acceptable salt or derivative thereof, wherein

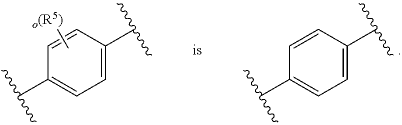

16. A compound selected from: 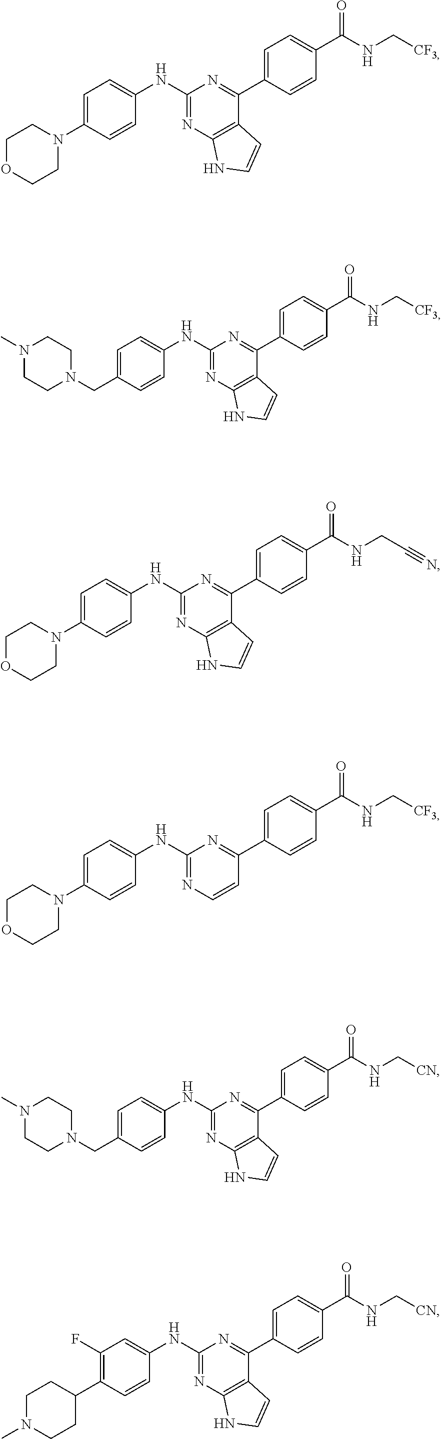 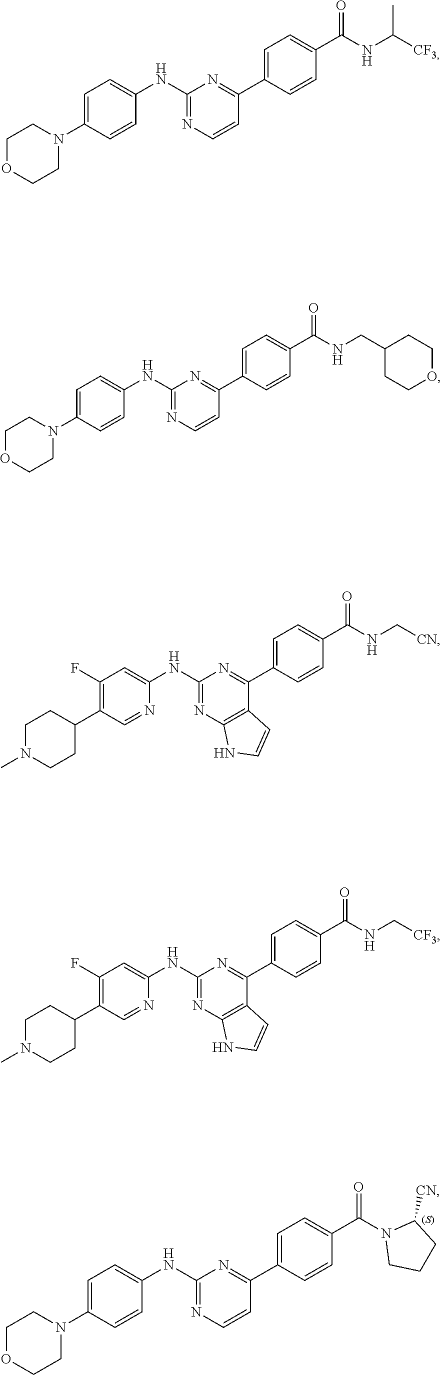 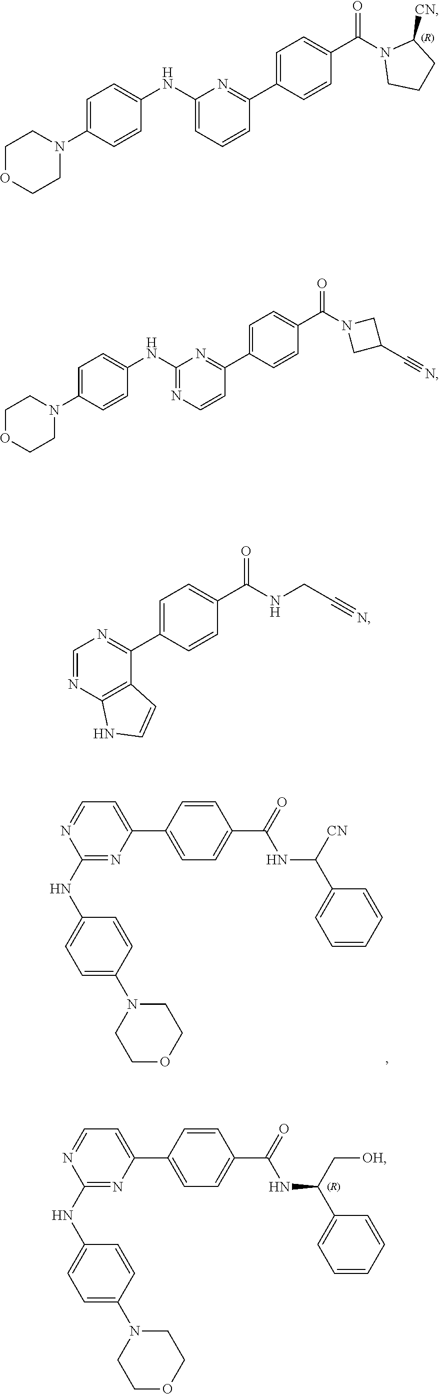 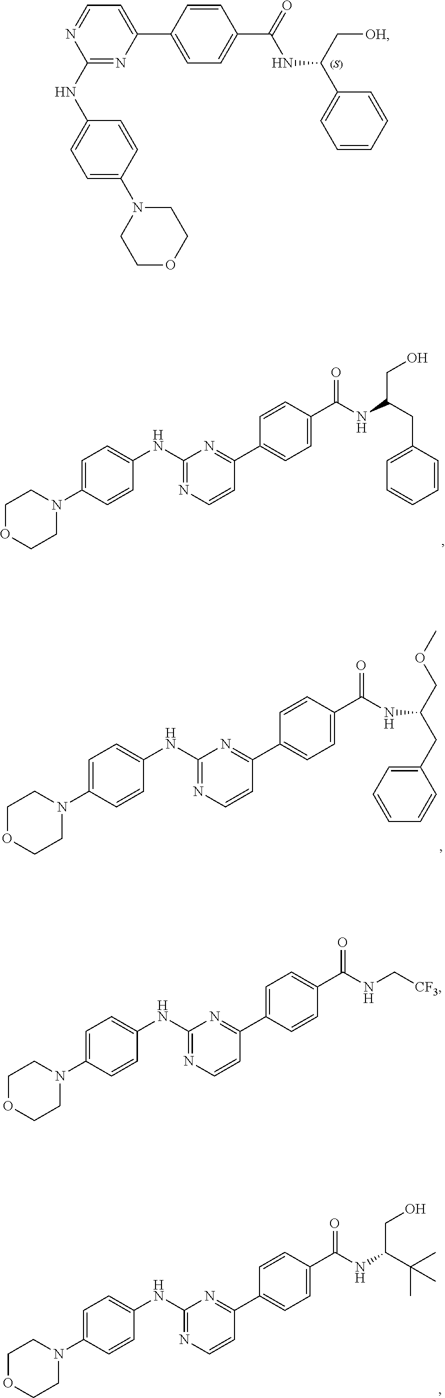 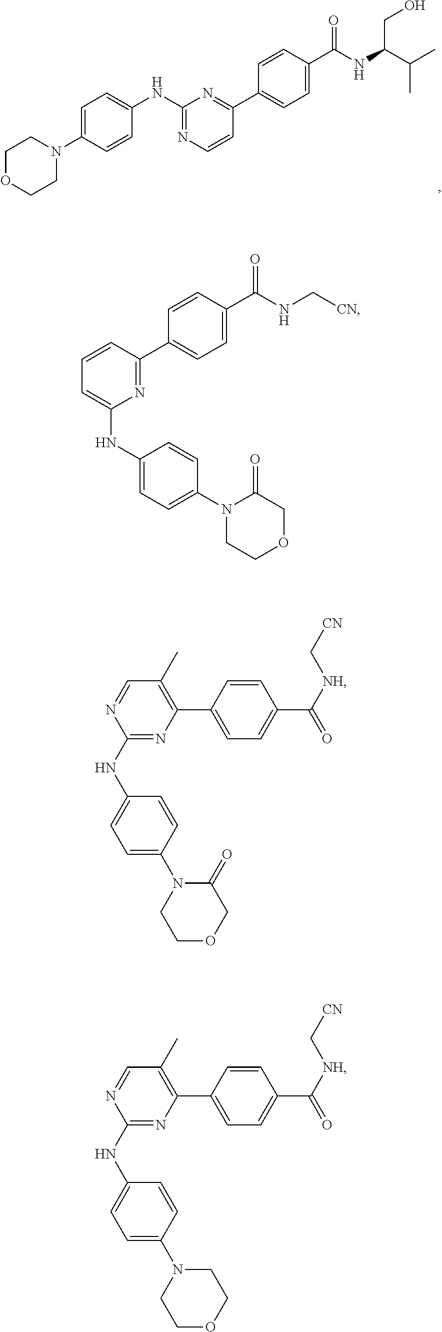 [SMILES: CC(C)[C@H](CO)NC(=O)c1ccc(-c2ccnc(Nc3ccc(N4CCOCC4)cc3)n2)cc1 ; N#CCNC(=O)c1ccc(-c2cccc(Nc3ccc(N4CCOCC4=O)cc3)n2)cc1 ; Cc1cnc(Nc2ccc(N3CCOCC3=O)cc2)nc1-c1ccc(C(=O)NCC#N)cc1 ; Cc1cnc(Nc2ccc(N3CCOCC3)cc2)nc1-c1ccc(C(=O)NCC#N)cc1] 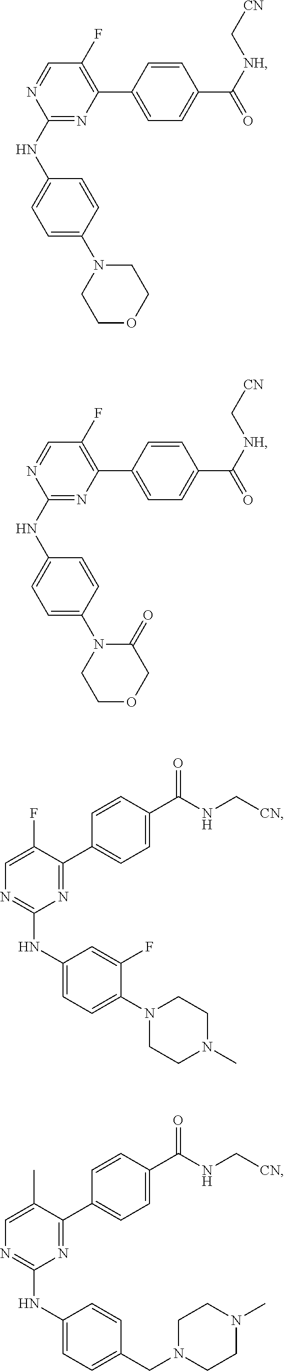 [SMILES: N#CCNC(=O)c1ccc(-c2nc(Nc3ccc(N4CCOCC4)cc3)ncc2F)cc1 ; N#CCNC(=O)c1ccc(-c2nc(Nc3ccc(N4CCOCC4=O)cc3)ncc2F)cc1 ; CN1CCN(c2ccc(Nc3ncc(F)c(-c4ccc(C(=O)NCC#N)cc4)n3)cc2F)CC1 ; Cc1cnc(Nc2ccc(CN3CCN(C)CC3)cc2)nc1-c1ccc(C(=O)NCC#N)cc1] 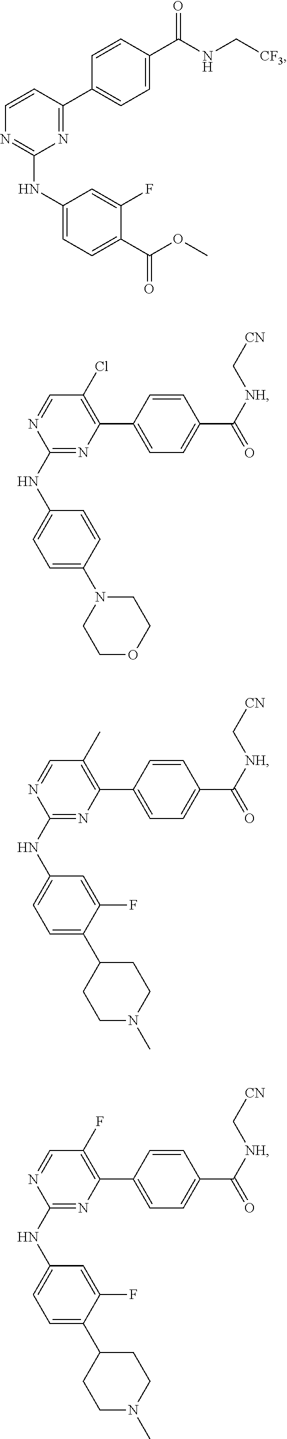 [SMILES: COC(=O)c1ccc(Nc2nccc(-c3ccc(C(=O)NCC(F)(F)F)cc3)n2)cc1F ; N#CCNC(=O)c1ccc(-c2nc(Nc3ccc(N4CCOCC4)cc3)ncc2Cl)cc1 ; Cc1cnc(Nc2ccc(C3CCN(C)CC3)c(F)c2)nc1-c1ccc(C(=O)NCC#N)cc1 ; CN1CCC(c2ccc(Nc3ncc(F)c(-c4ccc(C(=O)NCC#N)cc4)n3)cc2F)CC1] 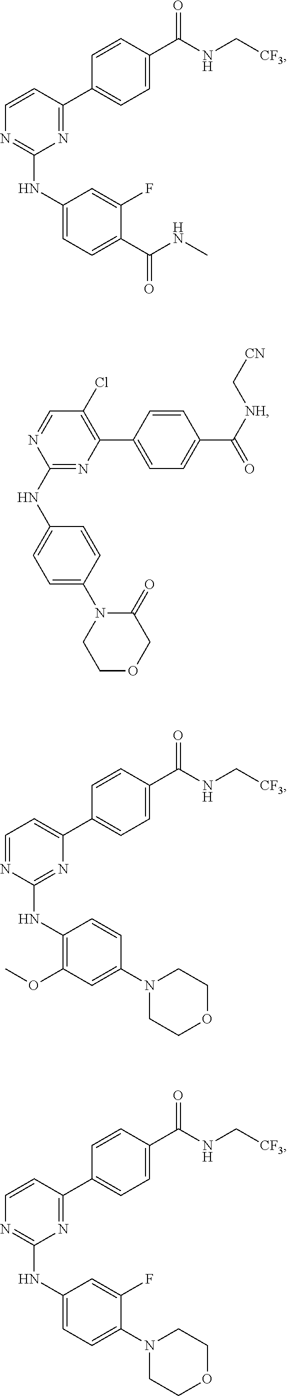 [SMILES: CNC(=O)c1ccc(Nc2nccc(-c3ccc(C(=O)NCC(F)(F)F)cc3)n2)cc1F ; N#CCNC(=O)c1ccc(-c2nc(Nc3ccc(N4CCOCC4=O)cc3)ncc2Cl)cc1 ; COc1cc(N2CCOCC2)ccc1Nc1nccc(-c2ccc(C(=O)NCC(F)(F)F)cc2)n1 ; O=C(NCC(F)(F)F)c1ccc(-c2ccnc(Nc3ccc(N4CCOCC4)c(F)c3)n2)cc1] 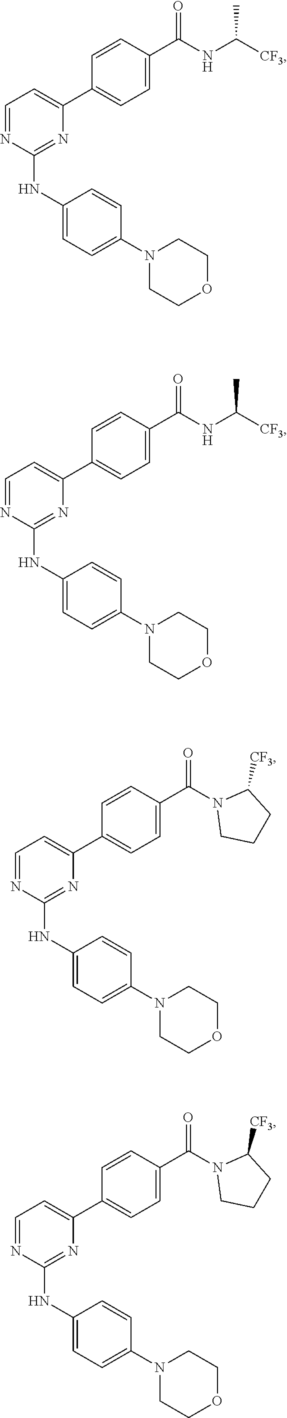 [SMILES: C[C@@H](NC(=O)c1ccc(-c2ccnc(Nc3ccc(N4CCOCC4)cc3)n2)cc1)C(F)(F)F ; C[C@H](NC(=O)c1ccc(-c2ccnc(Nc3ccc(N4CCOCC4)cc3)n2)cc1)C(F)(F)F ; O=C(c1ccc(-c2ccnc(Nc3ccc(N4CCOCC4)cc3)n2)cc1)N1CCC[C@H]1C(F)(F)F ; O=C(c1ccc(-c2ccnc(Nc3ccc(N4CCOCC4)cc3)n2)cc1)N1CCC[C@@H]1C(F)(F)F] 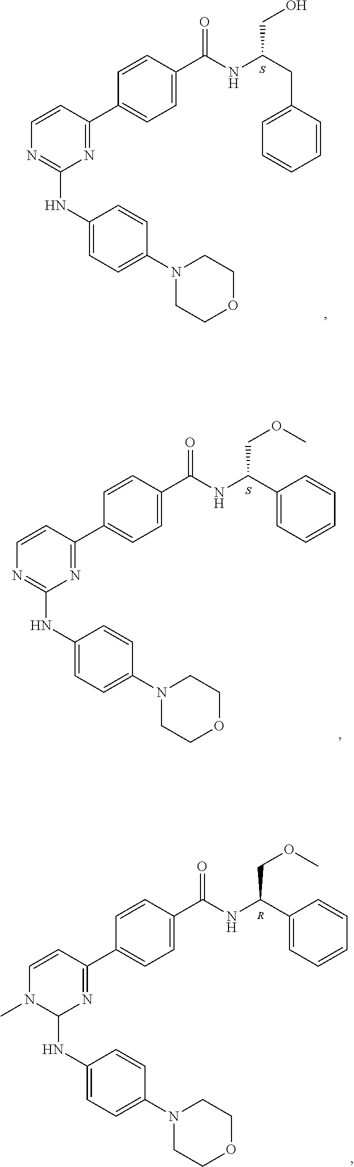 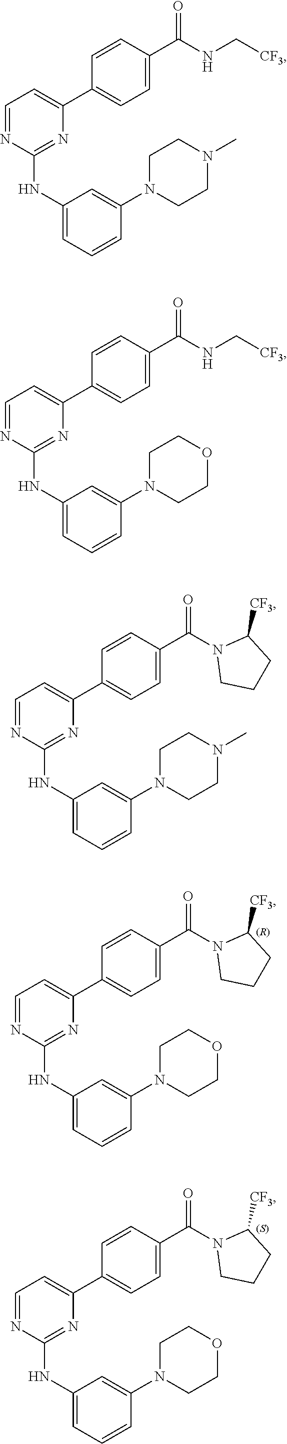 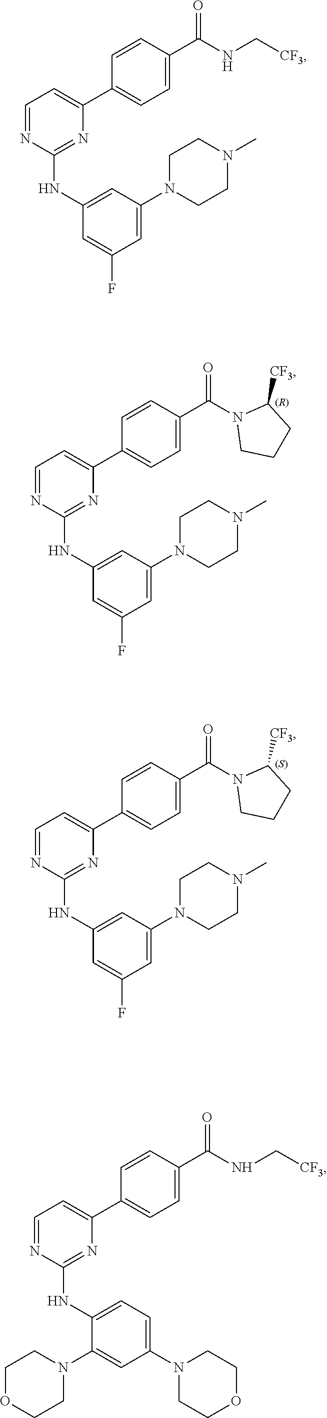 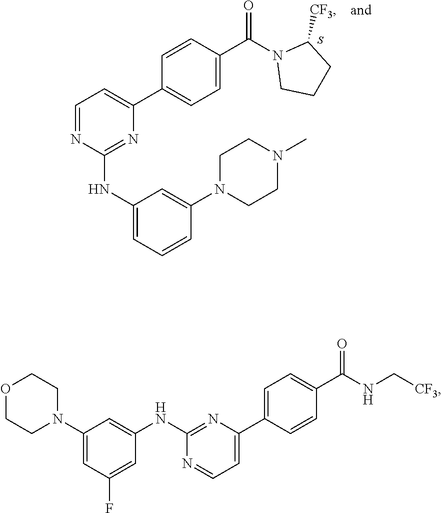
or a pharmaceutically acceptable salt or derivative thereof.

17. A pharmaceutical composition comprising a compound of claim 1, or a pharmaceutically acceptable salt or derivative thereof, and a pharmaceutically acceptable carrier or excipient.

18. A method of treating a cancer in a subject in need thereof comprising administering to the subject a therapeutically effective amount of a compound of claim 1, or a pharmaceutically acceptable salt or derivative thereof.

19. The method of claim 18, wherein the cancer comprises a ULK-associated cancer.

20. The method of claim 18, wherein the cancer comprises multiple myeloma or breast cancer.

21. (canceled)

22. A method of treating a ULK3-associated cancer in a subject in need thereof comprising administering to the subject a therapeutically effective amount of a compound of Formula I 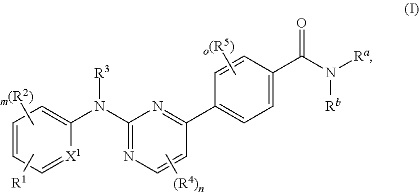
or a pharmaceutically acceptable salt or derivative thereof,
wherein:
m is 0, 1, 2, or 3;
n is 0, 1, or 2;
o is 0, 1, 2, 3, or 4;
R¹ is selected from —(C₀-C₃alkyl)(3- to 8-membered monocyclic or bicyclic heterocycle), R^{x}O—C(O)—(C₀-C₃ alkyl)-, and (R^{x}R^{y}N)—C(O)—(C₀-C₃ alkyl)-, each of which may be optionally substituted with one or more groups selected from Z as allowed by valency;
X¹ is selected from N or C(R²);
R², R⁴, and R⁵ are each independently selected at each occurrence from hydrogen, halo, cyano, azido, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂-(C₀-C₃ alkyl)-, (R^{x}R^{y}N) S(O)₂-(C₀-C₃ alkyl)-, R^{z}C(O)—O—(C₀-C₃ alkyl)-, R⁷C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂-(C₀-C₃ alkyl)-, each of which may be optionally substituted with one or more groups selected from Z as allowed by valency;
R³ is hydrogen or C₁-C₃ alkyl;
R^{a} and R^{b} are independently selected from hydrogen, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, and (5- to 10-membered monocyclic or bicyclic heteroaryl)-(C₀-C₃ alkyl)-, each of which may be optionally substituted with one or more groups selected from Z as allowed by valency;
or R^{a} and R^{b} are brought together with the nitrogen to which they are attached to form 3- to 8-membered monocyclic or bicyclic heterocycle optionally substituted with one or more groups selected from Z as allowed by valency;
Z is independently selected at each occurrence from hydrogen, halo, cyano, azido, oxo, C₁-C₆ alkyl, C₁-C₆ haloalkyl, C₂-C₆ alkenyl, C₂-C₆ alkynyl, (C₃-C₆ cycloalkyl) (C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂-(C₀-C₃ alkyl)-, (R^{x}R^{y}N) S(O)₂-(C₀-C₃ alkyl)-, R^{z}C(O)—O—(C₀-C₃ alkyl)-, R⁷C(O)—(R^{x}N)—(C₀-C₃ alkyl)-, R^{z}S(O)₂-O—(C₀-C₃ alkyl)-, R^{z}S(O)₂-(R^{x}N)—(C₀-C₃ alkyl)-, R^{z}C(O)—(C₀-C₆ alkyl)-, R^{z}S(O)—(C₀-C₃ alkyl)-, and R^{z}S(O)₂-(C₀-C₃ alkyl)-, each of which is optionally substituted with one or more groups selected from Y as allowed by valency;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol;
wherein the compound of Formula I is not momelotinib.

23-25. (canceled)

26. A pharmaceutical composition comprising a compound of claim 16, or a pharmaceutically acceptable salt or derivative thereof, and a pharmaceutically acceptable carrier or excipient.

27. A method of treating a cancer in a subject in need thereof comprising administering to the subject a therapeutically effective amount of a compound claim 16, or a pharmaceutically acceptable salt or derivative thereof.

28. The method of claim 27, wherein the cancer comprises a ULK-associated cancer.

29. The method of claim 27, wherein the cancer comprises multiple myeloma or breast cancer.
